(19) United States
(12) Patent Application Publication
(10) Pub. No.: US 2025/0006368 A1
(43) Pub. Date: Jan. 2, 2025

(54) SAMPLING INTERVAL ADJUSTED CLASSIFICATION TOOL

(71) Applicant: Universität Heidelberg, Heidelberg (DE)

(72) Inventors: Gesa Albert, Rotkreuz (CH); Evangelos Giannitsis, Heidelberg (DE); Andrea Horsch, Rotkreuz (CH); Hauke Hund, Heidelberg (DE); Hugo Katus, Heidelberg (DE); Kiril Stoyanov, Heidelberg (DE)

(21) Appl. No.: 18/692,301

(22) PCT Filed: Sep. 15, 2022
(86) PCT No.: PCT/EP2022/075713

§ 371 Date: Mar. 14, 2024

(30) Foreign Application Priority Data
Sep. 15, 2021 (EP) 21196961.3

(51) Int. Cl. G16H 50/20; G16H 10/40

(52) U.S. Cl. CPC G16H 50/20; G16H 10/40

(57) ABSTRACT

The present invention relates to methods for assessing subjects presenting with suspected acute coronary syndrome. Specifically, the present invention relates to methods for classifying a patient with suspected acute coronary syndrome. The methods of the present invention may be carried out as computer-implemented methods.

The present invention relates to methods for assessing subjects presenting with suspected acute coronary syndrome. Specifically, the present invention relates to methods for classifying a patient with suspected acute coronary syndrome. The methods of the present invention may be carried out as computer-implemented methods.

### BACKGROUND

In the United States, a total of 6.9 million patients visit an emergency department (ED) for chest pain (CP), and another 3.4 million for shortness of breath according to the National Hospital Ambulatory Medical Care Survey in 20141. Only a fraction of these patients have a final diagnosis of ACS and require hospitalization and an invasive treatment strategy. The diagnostic workup requires admission to an ED, registration of a 12-lead electrocardiogram (ECG), a blood test to diagnose or to exclude myocardial injury, assessment of clinical symptoms and history, physical examination, and other diagnostic tests for diagnosis of ACS or differential diagnoses. Current 2020 European Society of Cardiology (ESC) guidelines² recommend monitoring of patients for ECG and vital signs, unless a myocardial injury has been ruled out. In a trend analysis, numbers of patients seeking for medical attention in EDs for chest pain is increasing, while rates of confirmed MI are stable or slightly decreasing. This trend creates a disparity between monitoring capacities, staff capacities including physician time for attendance and numbers of patients waiting to be seen. This disparity is often referred to as crowding or overcrowding, although an established definition or mathematical equation that describes crowding is not available. ED crowding is linked to adverse patient outcomes, patient satisfaction and decrease in quality of care¹. One possible option to reduce or overcome crowding is acceleration of the diagnostic workup leading to an earlier patient disposition, i.e. the decision to admit or discharge, and a facilitation of the decision and the timing of coronary angiography with or without reperfusion therapies. Decision and timing of an invasive strategy depend on an individual risk stratification that includes consideration of variables associated with very high, high or low risk for death or MI. Use of a clinical multivariable score such as the GRACE score is recommended by ESC guidelines².

The introduction of high-sensitivity troponin assays has enabled a more precise diagnosis of small myocardial infarcts and an earlier diagnosis due to improvement of analytical sensitivity and assay precision. Previous recommendations to collect cardiac troponin I or T at presentation and after 6 to 9 hours have been replaced by the recommendation to measure cTnT or cTnI at presentation and at 3 hours, if a hs-cTn assay is available^{3,4}. More recently, faster protocols endorsing a re-testing of cTnT or cTnI within 120 minutes are being recommended by ESC guidelines² as an alternative option if hs-cTn assays are used that have been validated for this purpose. The use of hs-cTn assays not only accelerates diagnosis, but was shown to improve short- and long-term risk of death, because the magnitude of hs-cTn in blood reflects the amount of myocardial damage. The use of validated fast protocols, measurement of low hs-cTn values together with small concentration changes, or undetectable hs-cTn values (below the limit of detection) at presentation among patients presenting to an ED more than 3 hours after last episode of symptoms allow to rule-out an MI with a misclassification rate of 1% or less⁴. Fast protocols also indicate a risk for follow-up death below 1%⁵, and a low risk for a combined major adverse event such as death or MI. However, fast protocols bear the risk to miss a myocardial infarction and unwarranted discharge from hospital. Patients presenting with suspected ACS within 3 hours after chest pain onset are at particular risk to miss an MI due to a delay of cardiac troponin to appear in blood at relevant concentrations (“troponin blind period”). Therefore, a second blood draw after 60 minutes is mandatory for correct classification, if patients present within 3 hours after onset of symptoms. Cardiac troponin cutoffs and concentration changes in fast protocols have been constructed to yield optimal performance for rule-out and rule-in, thereby creating a greyzone where patients are neither classified into rule-out nor rule-in. This group is characterized by moderate elevation of cardiac troponin and only moderate concentration changes. For a final classification, the 2020 ESC Guidelines recommend a third blood draw at 3 hours. Although the advantages of accelerated diagnostic protocols are intriguing, global adoption of hs-cTn assays and implementation of fast protocols are lagging behind⁴. The most plausible reasons for the lack of implementation include lack of familiarization, particularly in the United States where hs-cTnT was not cleared by the FDA as the first hs-cTn assay in February 2017 and the fear of litigation in case of missed MI or death. Furthermore, data on the safety of discharge after rule-out mainly stems from observational registries where physicians were blinded to the results of hs-cTn assays and protocols, and where patients were treated at the discretion of the attending physicians³⁻⁵. Conversely, only sparse data is available from randomized intervention trials that tested the safety of hs-cTn in combination with validated clinical scores⁴. Recently, the usefulness of traditional risk indicators and the value of clinical scores—currently recommended for risk stratification and guidance of invasive strategy by the 2020 ESC guidelines⁶—has been questioned when hs-cTn assays are used at decision cutoffs below the 99^{th} percentile values^{3,4}. Accordingly, the TRAPID AMI study, the APACE study and the High-STEACS trial reported no improvement of safety but a decrease of effectiveness of the respective rapid protocol due to declining numbers of patients eligible for the rule-out strategy⁴.

Currently available protocols for the diagnosis of NSTE-ACS combine biomarker concentrations at presentation and at later time points. While the 2015 ESC guidelines⁶ recommend a re-testing of hs-cTn 3 hours after the baseline testing, faster protocols are being recommended by the 2020 ESC Guidelines² with re-testing after 1 or 2 hours. The elapsed time between blood samples is important as the presence and severity of acute myocardial injury is reflected by the magnitude of the baseline value and by relevant is concentration changes. The cutoff at baseline and the concentration change criteria are protocol dependent and are different for each commercially available cTn assay and for each time point of re-testing.

For example, the treating physician may implement the ESC 0/1-hour algorithm in the ED (Emergency department) as the standard protocol. For several reasons, including unexpected workload, emergencies, technical errors, or staff break, the time for re-testing may be not after approximately 60 minutes but after a tolerable time delay, e.g. >90 minutes. In another example, the nurse re-tests at 2 hours but does not inform the physician about the actual time interval between testing. Interpretation of the concentration change according to the ESC 0/1 hour protocol would be erroneous as larger concentration changes are accepted as not relevant if they occur within longer time intervals (1 hour delta x vs. 2 hour delta y for hs-cTnT). Hence, the number of patients qualifying for rule-out based on small concentration changes would decline.

A second challenge for the attending physician in the ED is the interpretation of troponin results, given that there is no standardization of hs-cTnI assays, and the cutoff values and concentration changes have to be validated for each commercially available assay. Thus, the definition criteria of the three diagnostic categories (rule-out, observe, rule-in) are specified differently depending on the assay and protocol used³. Moreover, rapid diagnostic protocols differ regarding timing of the second blood draw, and whether rapid protocols have to be used together with a clinical score (accelerated diagnostic protocols, ADP) instead of unstructured clinical assessment. Differences exist also regarding the recommendation to consider sex-specific cutoffs for men and women for diagnosis, and other patient characteristics that might influence baseline hs-cTn concentrations such as advancing age, pre-existing CAD, underlying structural heart disease, or chronic kidney disease. At present, at least five different diagnostic strategies are being used in clinical routine.

A third challenge for physicians in the ED is to cope with an increasing number of patients with unspecific chest pain that increase workload for phy<sicians and medical staff and decrease patient satisfaction due to prolonged waiting times. Fast protocols are particularly helpful to decrease congestion (“crowding”) in busy EDs by allowing a personalized, faster and more accurate classification, hereby improving earlier patient disposition, higher guideline adherence and cost efficiency.

The idea to reduce the number of strategies at an individual ED is challenging, since time intervals between blood draws may be confounded by crowding or infrastructural issues. The likelihood to miss a diagnosis due to incorrect interpretation of serial cTn due to human error or unawareness results is substantial, unless physicians receive interpretation support in form of pocket cards, posters, or electronic assistance. Accordingly, a previous publication introduced hardware and an electronic tool that enables diagnosis and prognostication of MACE with continuous values for baseline and follow-up hs-TnT or hs-cTnI⁷. Others have published a machine-learned algorithm on a software application that allows an individualized diagnosis of ACS and prediction of type 1 MI based on tree-based machine learning⁸⁴. The input variables comprise age, sex, paired high-sensitivity cardiac troponin I concentrations and rate of change of cardiac troponin concentrations⁸.

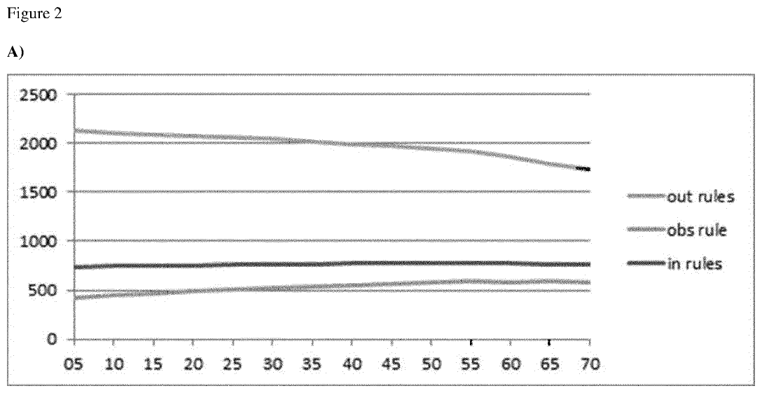

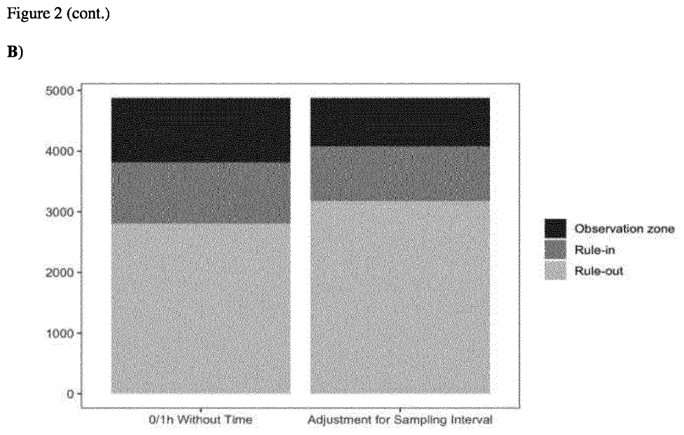

In the studies underlying the present invention, a method was developed which improves the assessment of patients presenting with suspected ACS (see also Examples section). Specifically, a classification method (herein also referred to as “method of the present invention”) was established which allows for the classification of rule-in, observe and rule-out for ACS based on different blood drawing schemes. The method not only integrates the calculation of different rules but also proposes optimal time-points with corresponding upper and lower intervals for the next blood draw and provides correct interpretation of findings based on the actual protocol used and not on an intended protocol. In addition, it provides information whether additional blood draws are required, e.g. a second blood draw after a diagnostic first sample, or a third blood draw if classification is not possible after two blood draws. The tool allows to follow whether ACS classification was performed correctly following guideline recommendations (quality control and guideline adherence) and to disclose at which point protocol violations occurred. Thus, the tool provides a step-up or step-down of an intended protocol, e.g. from a 0/1 hour to a 0/2 hour or to a 0/3 hour or vice versa. The classification method is advantageous because it allows to increase the proportion of patients with a rule-out diagnosis of myocardial infarction. As a consequence, a higher rate of ruled-out patients would increase the opportunity to discharge a substantial proportion of low-risk patients, and would decrease un-warranted treatments and unnecessary invasive procedures in patients assigned falsely to a rule-in or observe zone category (see FIG. 2A, a bar chart is shown in FIG. 2B).

Accordingly, the present invention relates to a computer-implemented method for classifying a patient with suspected acute coronary syndrome, comprising the steps of
(a) receiving, at a processing unit, information on a first time-point at which a first sample has been obtained from the patient at presentation,
(b) providing on a display
   b1) a proposal for a second time-point at which a second sample shall be obtained from the patient, wherein the second time point is within an interval of about 1 hour or of about 2 hours after the first time point, and
   b2) a proposal for an ACS classification algorithm to be applied for the classification of the patient, wherein the ACS classification algorithm is based on the second time point of proposed in step b1),
(c) receiving, at a processing unit:
   c1) information on the actual time-point at which the second sample has been obtained,
   c2) a value for the amount of the cardiac Troponin in the first sample, and
   c3) a value for the amount of the cardiac Troponin in the second sample
(d) carrying out by the processing unit an analysis on whether the second sample has been obtained within the interval under b1), wherein the sample is considered to have been obtained within the interval of about 1 hour, if it has been obtained between 30 to 90 minutes after the first sample, and/or wherein the second sample is considered to have been obtained within the interval of about 2 hours, if it has been obtained between 91 to 150 minutes after the first sample, and
(e) classifying the patient by the processing unit, wherein
   e1) the patient is classified based on the ACS classification algorithm proposed in step b2), if the second sample has been obtained within the interval proposed in step b1), or
   e2) wherein the patient is classified by an ACS classification algorithm which differs from the ACS classification algorithm proposed in step b2), if the sample has not been obtained within the interval proposed in step b1), and, optionally,
(f) providing information on the classification of the patient on a display.

The method of the present invention may comprise further steps. Such steps can be carried out before step a), within steps a) to f) of after step f).

### Selection of a Suitable Protocol for Classifying the Patient

For example, a suitable diagnostic protocol for the classification of the patient can be chosen, e.g. by the user, before carrying out step a) of the method of the present invention. Information on the selecting of the diagnostic protocol is, typically, received by the processing unit (as well as information on the diagnostic protocol). As known by the skilled person, different diagnostic protocols for the classification of patients with suspected acute coronary syndrome exist. Such protocols are well-known in the art and can be implemented in the method of the present invention. Preferably, the diagnostic protocol is a 0/1 hour, 0/2 hour, or a 0/3 hour protocol, in particular a 0/1 hour or 0/2 hour protocol. The protocols include, for example, information on the difference in times of troponin testing, information on cutoff values and concentration change values for Troponin for the classification. Further, the protocols include information on the timing of the last symptomatic episode (and how to use this timing for the classification). The cutoff values and concentration change values might dependent on the protocol. In some embodiments, the cutoff values and concentration change values indicative for the classification depend on the assays, e.g. there could be assay specific values. Further, the protocols may have sex-specific cutoff values for males and females. Alternatively, the diagnostic protocols may comprise sex-independent cutoff values for cardiac Troponins.

For US application, the FDA refuses reporting of high sensitivity cardiac troponin at the limit of detection due to inappropriate precision. Therefore, the lowest concentration that can be reported is at the limit of blank that allows measurement of cardiac troponin with an imprecision of 10% or less. In addition, the FDA recommends the use of sex-specific cutoffs for males and for females instead of a general, sex-independent 99^{th} percentile upper limit of normal. For example, the values for limit of reporting, single 99^{th} percentile upper limit of normal, the sex-specific cutoff for males and females are 6 ng/L, 19 ng/L, 22 and 14 ng/L, respectively for the high-sensitivity cardiac troponin T assay from Roche Diagnostics. Because high-sensitivity cardiac troponin T had been used in many US hospitals before its FDA clearance 2017, many clinicians prefer to triage patients according to the criteria proposed by the ESC. Others dislike the inconvenient implementation of sex-specific 99^{th} percentile upper limit of normal cutoff but prefer the use of the single cutoff that has been proposed by the FDA. Accordingly, there is heterogeneity regarding the implementation of US specific diagnostic protocols but also regarding the use of sex-specific cutoffs versus a single sex-independent cutoff in the US.

Accordingly, the method of the present invention may start with the selection of a suitable protocol for the classification of the subject. This, step is typically carried out prior to step a). For example, a US version (such as a FDA recommended protocol) or a non-US version (such as a protocol recommended by the ESC). Further, sex-specific cutoffs or sex-independent cutoffs could be selected. As set forth above, such protocols are well known in the art. Exemplary protocols are shown in Tables C1, C2 and C3 below. In some embodiments, the protocol is a protocol shown in these tables.

Further Classification of the Patient (if Classified into the “Observational Zone”)

Moreover, additional steps can be carried out after carrying out the above method. For example, a third blood draw might required for patients classified into the “observational zone” after the second sample (in step e). Such patients could be further classified based on a third blood sample (preferably by the processing unit)

Thus, step f) might be as follows.

f) providing information on the classification of the patient on a display and optionally information on whether a third sample is necessary for further classification of the patient. Typically, the information is provided by the processing unit.

Typically, a third sample is necessary, if the patient is classified into the “observational zone” after the second sample, i.e. if an ACS classification is not possible after the second blood sample because the patient neither meets criteria for rule-out or rule-in. Typically, a third sample is not necessary, if the patient is classified as rule-out or rule-in after the second sample.

If the patient is classified into the “observational zone”, step f) may further comprise, providing, e.g. on the display, a proposal for a third time point at which a third sample shall be obtained from the patient, wherein the third time point is 3 hours or later after the first sample.

A subsequent step g) preferably, comprises, receiving, at a (the) processing unit:
g1) information on the actual time-point at which the third sample has been obtained; and
g2) a value for the amount of the cardiac Troponin in the third sample.

A subsequent step h), preferably, comprises:
(h) classifying the patient by the processing unit as rule-out or rule-in. Preferably, the patient is classified as rule-out or rule-in based on the value of the cardiac Troponin in the third sample, in particular based on the difference between the value in the first and the third sample.

Preferably, the classification is only made, if third sample has been obtained at 3 hours or later after the first sample. This can be calculated by the processing unit. If the third sample has been obtained less than after the first sample, information can be provided in the display that no further classification is possible (violation of the protocol, see also next paragraph).

### Providing Information on Non-Adherence of the Protocol

In an embodiment, the tool, i.e method of the present invention, provides transparent information on protocol/Guideline adherence and protocol violations, i.e. a) missing second or third blood draw that is required and recommended by Guidelines, and b) excessive blood draws beyond the protocol requirement causing higher labor and laboratory costs, additional unnecessary observation times, unnecessary delays to disposition, i.e. admission to hospital, discharge or referral, indication and timing of coronary angiography, additional diagnostic workup. In case the protocol is violated, e.g. because a required second or third sample is missing, the physician can be informed on the violation (e.g. by a warning sign on the display and/or an audio warning). For example, a second blood draw that is required for rule-out in early presenters (less than 3 our after onset of chest pain before taking the first sample) might be missing. For example, a third blood draw required for patients classified into the “observational zone” after the second sample might be missing. In contrast, additional blood draws beyond protocol requirements could be avoided. Of note, additional blood draws are not necessarily inappropriate but should be ordered at the discretion of the attending physician whenever there are residual doubts about the correct classification with standard protocols.

### Additional Steps

Preferably, step (b) of the method of the present invention may further comprise: providing on a display information on whether a second sample is necessary, or not, for classifying the patient. For example, if the cardiac troponin concentration in the first sample is below LoD and if chest pain onset is known and equal or greater to 3 hours before the first sample was obtained, the patient is classified as “rule-out”, i.e. a myocardial infarction is ruled out. In contrast a troponin equal or above 52 ng/L in the sample classifies the patient into instant as “rule-in”. Thus, a second sample is only necessary, if the patient cannot be classified based on the amount of the cardiac Troponin in the first sample. In other words, a second sample is not necessary, if the patient can be classified as “rule out” or “rule in” based on the first sample. The information on whether a second sample is necessary, or not, allows for avoiding an unnecessary second blood draw, thereby reducing staff time and laboratory costs, reduce length of stay in ED, expedite earlier discharge after rule-out, or facilitate the indication for an invasive strategy in case of rule-in (such as coronary angiography).

Preferably, the proposals in steps b1) and b2) are made, if a second sample is necessary, i.e. if the patient cannot be classified (as “rule out” or “rule in”) based on the amount of a cardiac Troponin in the first sample. If the patient is ruled in or ruled out based on the amount of the first sample, information on the classification of the patient can be provided on a display. In this case, it is not required carry out the further steps of the method of the present invention. In an embodiment, however, the patient to be tested is a patient who cannot be classified as “rule in” or “rule out” based on the first sample.

Accordingly, step b) of the method of the present invention, preferably, is only carried out in case that the patient cannot be classified based on the first sample onl. Thus, step b may be as follows:
b) providing on a display
   b1) a proposal for a second time-point at which a second sample shall be obtained from the patient in case that the patient cannot be classified based on the first sample only, wherein the second time point is within an interval of about 1 hour or of about 2 hours after the first time point, and
   b2) a proposal for an ACS classification algorithm to be applied for the classification of the patient, wherein the ACS classification algorithm is based on the second time point of proposed in step b1), in case that the patient cannot be classified based on the first sample only.

Thus, in some instances, the patient can be classified based on the first sample already (although it is envisaged that the test subject cannot be classified based on the first sample already).

Accordingly, step e) of the method of the present invention might be as follows:
(e) classifying the patient by the processing unit, wherein
   e1) the patient is classified based on the ACS classification 0/1 hour algorithm, if the hs-cTn value derived under c2) is below the assay specific immediate rule-out cutoff or above the assay specific immediate rule-in cutoff
   e2) the patient is classified based on the ACS classification algorithm proposed in step b2), if the second sample has been obtained within the interval proposed in step b1), or
   e3) wherein the patient is classified by an ACS classification algorithm which differs from the ACS classification algorithm proposed in step b2), if the sample has not been obtained within the interval proposed in step b1),

In a preferred embodiment of the aforementioned method, the second time-point proposed in step b1) is within an interval of about one hour after the first sample, and wherein the ACS classification algorithm proposed in step b2) is a 0/1 hour algorithm.

In a preferred embodiment of the aforementioned method, the ACS classification algorithm to be applied in step e2) is a 0/2 hour algorithm, if the second sample has been obtained within 91 to 150 minutes after the first sample.

In a preferred embodiment of the aforementioned method, the ACS classification algorithm to be applied in step e2) is a 0/3 hour algorithm, if the second sample has been obtained within 151 to 210 minutes after the first sample.

In a preferred embodiment of the aforementioned method, the ACS classification algorithm to be applied is sampling of a third sample at 3 hours or later after the first sample if the second measurement of the 0/1 or 0/2 hour algorithm is not able to rule-out or rule-in.

In a preferred embodiment, the subject is a human subject.

In a preferred embodiment, the sample is a blood, serum or plasma sample.

In a preferred embodiment, the subject is a subject presenting with symptoms of ACS at the emergency department.

The present invention further relates to a method for patient management in an emergency department having a plurality of patients with suspected ACS per day, said method comprising carrying out for each of the said patients steps a) to f) of the method of claim 1, thereby identifying patients
i) in which the diagnosis of myocardial infarction can be ruled in (rule-in),
ii) in which the diagnosis of myocardial infarction can be ruled out (rule-out), and
iii) who require further examination for ruling in or ruling out myocardial infarction (observation zone).

The present invention further relates to method for increasing the proportion of patients with a rule-out diagnosis of myocardial infarction within a plurality of patients with suspected ACS, wherein said method comprises carrying out steps a) to f) of claim 1 for said plurality of patients.

### Definitions

As the forth above, the present invention encompasses three methods, a prognostic method, a predictive method and a classification method. The definitions and explanations provided herein above shall apply to all methods, except if specified otherwise.

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of +20%, preferably +15%, more preferably ±10%, and even more preferably +5%. In an embodiment, the term refers to the exact value.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

It will be understood that the methods according to the present invention are, preferably, ex-vivo methods, i.e. they do not require to be practiced on the human or animal body. Rather, the methods are based on existing patient data previously gathered. For example, it is envisaged that the methods are in vitro methods. Moreover, they may comprise steps in addition to those explicitly mentioned above. For example, further steps may relate to sample pre-treatments or evaluation of the results obtained by the method. The method may be carried out manually or assisted by automation.

In some embodiments, the methods of the present invention are computer-implemented methods. In computer-implemented methods, typically, all steps of the computer-implemented method of the present invention are performed by one or more processing units of a computer or a computer network. However, the computer-implemented method may comprise additional steps, such as the determination of the amount of a marker in a sample, such as the amount of a cardiac Troponin in the first and the second sample.

The term “classifying” as used herein refers to allocating the patients into a) a group of patients suffering from acute coronary syndrome (“rule in”), b) a group of patients not suffering from acute coronary syndrome (“rule out”), or a group of patients which require further assessment in order to rule in or rule out myocardial infarction (“observe zone” or observation zone). Thus, a patient who is classified as “observe” needs a further assessment. For example, a value of a cardiac Troponin in a third sample, such as a sample obtained about three hours after the first sample, or later, may be provided.

Based on the classification, suitable measures, such as diagnostic or therapeutic measures, can be initiated, e.g. those described by the 2020 ESC guidelines² (incorporated herein by reference), for example certain invasive measures for patients that are ruled in.

As will be understood by those skilled in the art, the aforementioned assessment made by the methods of the present invention, i.e. classification, are usually not intended to be correct for 100% of the investigated individuals. The term typically requires that the assessment is correct for a statistically significant portion of the individuals (e.g., a cohort in a cohort study). Whether a value indicating a difference in risk or likelihood, a portion of a cohort or any other difference in values is statistically significant can be determined without further ado by the person skilled in the art using various well-known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001.

The term “sample” refers to a sample of a body fluid, to a sample of separated cells or to a sample from a tissue or an organ which is known or suspected to comprise an analyte which needs to be determined as a parameter. It will be understood that the sample may depend on the analyte to be determined. For example, if a cardiac Troponin shall be determined in a first and/or second sample as referred to herein, said sample may be typically a sample containing or suspected to contain said cardiac Troponin. Typical samples may be whole blood samples or derivatives thereof such as plasma or serum samples. For other analytes, the sample may be urine samples as well or other body fluids or cell or tissue samples. The skilled artisan is well aware which samples can be used for a given analyte in order to determine the parameter referred to in accordance with the present invention. Moreover, the skilled person is also well aware of how such samples can be taken from the patient, e.g., by conventional blood taking equipment such as lancets, biopsies or the like. The term “sample” and “blood draw” are used interchangeably herein.

In a preferred embodiment, the sample is blood, serum or plasma sample.

The term “cardiac Troponin” typically refers to human cardiac Troponin T or cardiac Troponin I. The term, however, also compasses variants of the aforementioned specific Troponins, i.e., preferably, of cardiac Troponin I, and more preferably, of cardiac Troponin T. Such variants have at least the same essential biological and immunological properties as the specific cardiac Troponins. In particular, they share the same essential biological and immunological properties if they are detectable by the same specific assays referred to in this specification, e.g., by ELISA Assays using polyclonal or monoclonal antibodies specifically recognizing the said cardiac Troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least about 50%, at least about 60%, at least about 70%, at least about 80%, at least about 85%, at least about 90%, at least about 92%, at least about 95%, at least about 97%, at 10 least about 98%, or at least about 99% identical with the amino sequence of the specific Troponin. Variants may be allelic variants or any other species specific homologs, paralogs, or orthologs. Moreover, the variants referred to herein include fragments of the specific cardiac Troponins or the aforementioned types of variants as long as these fragments have the essential immunological and biological properties as referred to above. Preferably, the cardiac troponin variants have immunological properties (i.e. epitope composition) comparable to those of human troponin T or troponin I. Thus, the variants shall be recognizable by the aforementioned means or ligands used for determination of the concentration of the cardiac troponins. Thus, the variants shall be recognizable by the aforementioned means or ligands used for determination of the concentration of the cardiac troponins. Such fragments may be, e.g., degradation products of the Troponins. Further included are variants which differ due to posttranslational modifications such as phosphorylation or myristylation. Preferably the biological property of troponin I and its variant is the ability to inhibit actomyosin ATPase or to inhibit angiogenesis in vivo and in vitro, which may e.g. be detected based on the assay described by Moses et al. 1999 PNAS USA 96 (6): 2645-2650). Preferably the biological property of troponin T and its variant is the ability to form a complex with troponin C and I, to bind calcium ions or to bind to is tropomyosin, preferably if present as a complex of troponin C, I and T or a complex formed by troponin C, troponin I and a variant of troponin T. Troponin T or Troponin I can be determined by immunoassays, e.g., ELISAs, that are well known in the art and commercially available. Particularly preferred in accordance with the present invention is the determination of Troponin T with high sensitivity using, e.g. a commercially available hs-cTnT assay. Alternatively, a high sensitivity Troponin I (hs-cTnI) may be used. hs-cTnT and hs-cTnI assays are known in the art and are disclosed, e.g. by Shah et al (Lancet. 2018 Sep. 15; 392(10151):919-928. doi: 10.1016/S0140-6736(18)31923-8. Epub 2018 August 28. PMID: 30170853; PMCID: PMC6137538) and by Mueller C, et al. (Ann Emerg Med. 2016; 68:76-87) which are both herewith incorporated by reference in its entirety.

In a preferred embodiment, the cardiac Troponin is cardiac Troponin T. In another preferred embodiment, the cardiac Troponin is cardiac Troponin I.

The term “amount” as used herein refers to the absolute amount of a compound referred to herein, the relative amount or concentration of the said compound as well as any value or parameter which correlates thereto or can be derived therefrom. Such values or parameters comprise intensity signal values from all specific physical or chemical properties obtained from the said compounds by direct measurements, e.g., intensity values in mass spectra or NMR spectra. Moreover, encompassed are all values or parameters which are obtained by indirect measurements specified elsewhere in this description, e.g., response levels determined from biological read out systems in response to the compounds or intensity signals obtained from specifically bound ligands. It is to be understood that values correlating to the aforementioned amounts or parameters can also be obtained by all standard mathematical operations.

The terms “determining” or “measuring” the level of a marker as referred to herein refers to the quantification of the biomarker, e.g. to determining the level of the biomarker in the sample, employing appropriate methods of detection described elsewhere herein. In an embodiment, the level of at least one biomarker is measured by contacting the sample with a detection agent that specifically binds to the respective marker, thereby forming a complex between the agent and said marker, detecting the level of complex formed, and thereby measuring the level of said marker.

The “patient” or “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the patient or subject in accordance with the present invention is a human. The patient referred to in accordance with the present invention shall be a patient presenting with suspected acute coronary syndrome (ACS), preferably at the emergency department. Typically, such a patient shall either suffer from ACS or shall exhibit at least one or more symptoms accompanying ACS, preferably chest pain. In particular, the subject shall suffer from acute chest pain.

The term “acute coronary syndrome (ACS)” as used herein refers to an obstructive event affecting coronary vessels involving multiple interrelated mechanisms. Preferably, in ACS a plaque may rupture or erode, in response to inflammation, leading to local occlusive or non-occlusive thrombosis. Depending on the degree and reversibility of this dynamic obstruction, the clinical manifestations of ACS comprise a continuous spectrum of risk that progresses from unstable angina (UA) to non-ST-segment elevation myocardial infarction (NSTEMI) to ST-segment elevation myocardial infarction (STEMI). NSTEMI is distinguished from UA by ischemia sufficiently severe in intensity and duration to cause myocyte necrosis, which is recognized by the detection of cardiac Troponins, the most sensitive and specific biomarker of myocardial injury. ACS is typically accompanied by prolonged chest pain episodes, preferably, 20 min or longer.

In a preferred embodiment, the patient to be tested is suspected to suffer from non-ST-segment elevation myocardial infarction (NSTEMI). Thus, the patient does not suffer from ST-segment elevation myocardial infarction (STEMI). STEMI is defined in the presence of persisting ST segment elevations in at least 2 contiguous leads or a new bundle branch block (right or left bundle branch block) or a permanently paced rhythm. A subject who is suspected to suffer from NSTEMI, preferably, has a negative ECG and, thus, does not have such ST segment elevations.

The term “data” as used herein refers to digital information such as numerical values indicative for the parameters of the set of parameters for which data shall be received in accordance with the present invention. Preferably, the digital numerical values shall represent amounts of compounds to be considered or counts of blood cells or thrombocyte level.

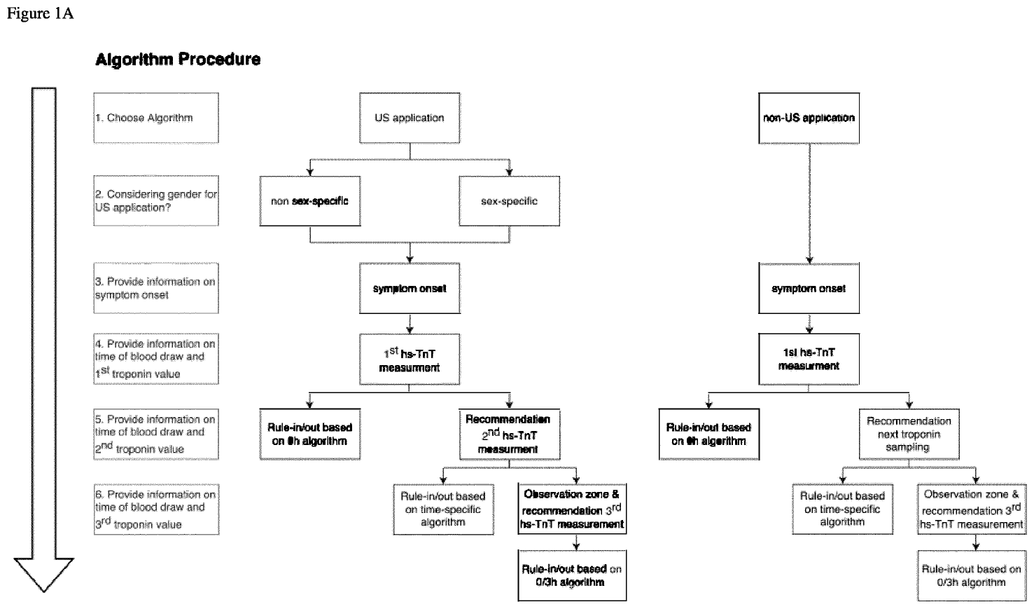

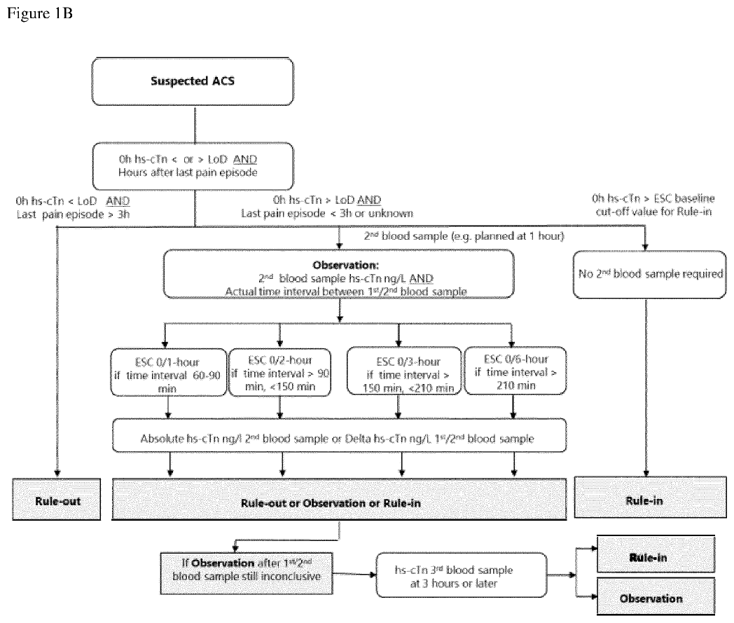

As set forth above, the classification method comprises steps a) to f). The method of the present invention is a computer-implemented method. Typically, all steps of the computer-implemented method of the present invention are performed by one or more processing units of a computer or a computer network. However, the computer-implemented method may comprise additional steps, such as the determination of the amount of a cardiac Troponin in the first and the second sample. The method can be carried out as depicted in FIG. 1 A or B.

In accordance with the classification method, the patient to the tested is a patient who presents with suspected acute coronary syndrome, e.g. at the emergency department. At presentation, a sample is taken from the patient.

Step a) of the classification method comprises receiving, at a processing unit, information on a first time-point at which a first sample has been obtained from the patient at presentation. Thus, the first sample preferably has been obtained at presentation.

In step b), the following information is provided on a display:
b1) a proposal for a second time-point at which a second sample shall be obtained from the patient, wherein the second time point is within an interval of about 1 hour or of about 2 hours after the first time point,
b2) a proposal for an ACS classification algorithm to be applied for the classification of the patient, wherein the ACS classification algorithm is based on the second time point of proposed in step b1).

In a preferred embodiment, an interval of about 1 hour is proposed in step b1). Thus, it is proposed to obtain the second sample about 1 hour (60 minutes) after the first sample has been obtained. In this case, a 0/1 hour protocol is proposed in step b2), such as the 0/1 hour protocol of the European Society of Cardiology (ESC) as disclosed in Collet J P, et al. (Eur Heart J. 2020 Aug. 29:ehaa575. doi: 10.1093/eurheartj/ehaa575 and in Pickering et al. Circulation. 2016; 134:1532-1541 which are both incorporated by reference with respect to there entire disclosure content.

The cutoff values or the concentration changes (between the first and second or the second and the third sample) indicative for the classification may depend on the protocol chosen for the classification. Tables C1, C2 and C3 show values for different protocols (such as 0/1 hour, 0/2 hour protocols) recommended by the ESC and the FDA, respectively. The values are for Roche hs-cTnT assay. Suitable values for other Troponin assays have been established or can be established without further ado.

With respect to cardiac Troponin T, preferably, the following applies for the 0/1 hour protocol.

An amount of cardiac Troponin T of lower than 12 ng/l in the first sample and a difference between the amount in the second sample to the amount in the first sample of less than 3 ng/l is indicative for the rule out of ACS. A difference between the amount of cardiac Troponin T in the first sample and the second sample equal to or larger than 5 ng/l is indicative for the rule in of ACS.

Moreover, a subject is considered to require further assessment for the classification (observation zone), if the subject has an amount of cardiac Troponin T of equal to or larger than 12 ng/l in the first sample and if the difference between the amount in the second sample and the first sample is equal to or larger than 3 ng/l, but lower than 5 ng/l. The above values e.g. apply to the Elecsys® Troponin T-high sensitive from Roche (Roche hsTnt).

With respect to Abbott Architect high sensitivity cardiac Troponin I, preferably, the following applies for the 0/1 hour protocol.

An amount of cardiac Troponin I of lower than 5 ng/l in the first sample and a difference between the amount in the second sample and the first sample of less than 2 ng/l is indicative for the rule out of ACS. A difference between the amount of cardiac Troponin I in the first sample and the second sample equal to or larger than 6 ng/l is indicative for the rule in of ACS.

Moreover, a subject is considered to require further assessment for the classification (observation zone), if the subject has an amount of Troponin I, such as the Abbott Architect high sensitivity cardiac Troponin I of equal to or larger than 5 ng/l in the first sample and if the difference between the amount in the second sample and the first sample is equal to or larger than 2 ng/l, but lower than 6 ng/l.

The above values e.g. apply to the Troponin I-high sensitive assay from Abbott (Architect). The 0/1 hour protocol for cardiac Troponin T is further described in Table C1, C2 and C3. below.

Other assays might be used as well for the 0/1 hour interval as well. Here the following applies;

An amount of cardiac Troponin of lower than A ng/l in the first sample and a difference between the amount in the second sample and the first sample of less than B ng/l is indicative for the rule out of ACS. A difference between the amount of cardiac Troponin I in the first sample and the second sample equal to or larger than C ng/l is indicative for the rule in of ACS.

Moreover, a subject is considered to require further assessment for the classification (observation zone), if the subject has an amount of cardiac Troponin of equal to or larger than A ng/l in the first sample and if the difference between the amount in the second sample and the first sample is equal to or larger than B ng/l, but lower than C ng/l.

Values for the parameters A, B and C for the individual assays are shown in the following Table A.

TABLE A
Parameters A, B and C (values are in [ng/l]

| Assay | A | B | C |
|---|---|---|---|
| hs-TnI (Architect; Abbott) | 5 | 2 | 6 |
| hs-TnI (ADVIA Centaur; Siemens) | 6 | 3 | 12 |
| hs-TnI (Access; Beckman Coulter) | 5 | 4 | 15 |
| hs-TnI (VITROS; Ortho Clinical Diagnostics)) | 2 | 1 | 4 |

In another preferred embodiment, an interval of about 2 hours is proposed in step b1). Thus, it is proposed to obtain the second sample about 2 hours (120 minutes) after the first sample has been obtained. In this case, a 0/2 hour protocol is proposed in step b2), such as the 0/2 hour protocol as disclosed in Reichlin (Am J Med. 2015 April; 128(4):369-79). The 0/2 hour protocol for TnT is further described in Table C1, C2 and C3 below Adapted from Collet J P et al. (2020). Eur Heart J00,1-79.

Value for the parameters can be found in Table B:

TABLE B
0/2 h algorithm (values are in [ng/l])

| | Very low | A | B | High | C |
|---|---|---|---|---|---|
| hs-cTn T (Elecsys; Roche) | <5 | 14 | 4 | >52 | 10 |
| hs-cTn I (Architect; Abbott) | <4 | 6 | 2 | >64 | 15 |
| hs-cTn I (Centaur; Siemens) | <3 | 8 | 7 | >120 | 20 |
| hs-cTn I (Access; Beckman Coulter) | <4 | 5 | 5 | >50 | 20 |

With respect to cardiac Troponin T, preferably, the following applies for the 0/2 hour algorithm:

An amount of cardiac Troponin T of lower than 14 ng/l in the first sample and a difference between the amount in the second sample to the amount in the first sample of less than 4 ng/l is indicative for the rule out of ACS. A difference between the amount of cardiac Troponin T in the first sample and the second sample equal to or larger than 10 ng/l is indicative for the rule in of ACS.

Moreover, a subject is considered to require further assessment for the classification (observation zone), if the subject has an amount of cardiac Troponin T of equal to or larger than 14 ng/l in the first sample and if the difference between the amount in the second sample and the first sample is equal to or larger than 4 ng/l, but lower than 10 ng/l.

After the first sample, a second sample shall be obtained from the subject. In an embodiment, the second sample is obtained within the time interval proposed in step b1). Preferably, however, the second sample is not obtained within the time interval proposed in step b2).

In step c) of the classification method, the following information is obtained at the processing unit:
c1) information on the actual time-point at which the second sample has been obtained,
c2) a value for the amount of the cardiac Troponin in the first sample, and
c3) a value for the amount of the cardiac Troponin in the second sample.

Subsequently, step d) of analyzing by the processing unit whether the second sample has been obtained within the interval under b1) is carried out. Preferably, the sample is considered to have been obtained within the interval of about 1 hour, if it has been obtained between 30 to 90 minutes, i.e. 60 min (+30 min or −30 min), after the first sample. Also preferably, the second sample is considered to have been obtained within the interval of about 2 hours, if it has been obtained between 91 to 150 minutes after the first sample.

Accordingly, a second sample which has been obtained more than 90 minutes after the first sample (such as between 91 to 150 minutes after the first sample) is considered to have not been obtained within an interval of about 1 hour. Further, a second sample which has been obtained more than 150 minutes after the first sample (such as between 151 to 210 minutes after the first sample) is considered to have not been obtained within an interval of about 2 hours.

In accordance with the present invention, it is in particular envisaged that the test subject is a patient whose second sample is considered to have not been obtained within the time interval proposed in step b). For example, the second sample may have been obtained at a time point which is later than the time point proposed in step b).

Preferably, the test subject may be a subject whose second sample has been obtained more than 90 minutes after the first sample (such as between 91 to 150 minutes) after the first sample, if the 0/1 hour algorithm has been proposed in step b1.

Preferably, the subject to be tested may be a subject whose second sample has been obtained after 151 minutes (if the 0/2 hour algorithm has been proposed in step b1).

An interval of less than 30 minutes is an insufficient time interval between blood samples.

Step e) of the classification method comprises classifying the patient by the processing unit. Preferably, the patient is classified based on the ACS classification algorithm proposed in step b2), if the second sample has been obtained within the interval proposed in step b1).

More preferably, the patient is classified by an ACS classification algorithm which differs from the ACS classification algorithm proposed in step b2), if the sample has not been obtained within the interval proposed in step b1). Therefore, the present invention may encompass the step of selecting a patient whose second sample has not been obtained within the time interval proposed in step b1).

For example, the second time-point proposed in step b1) may be within an interval of about one hour after the first sample, and the ACS classification algorithm proposed in step b2) is a 0/1 hour algorithm. As set forth above, the sample is considered to have been obtained within the interval of about 1 hour, if it has been obtained between 30 to 90 minutes (and thus 60 minutes (allowing—30 to +30 minutes)) after the first sample.

However, the sample is not considered to have been obtained within this time interval, if it has been obtained more than 90 minutes after the first sample. For example, the second sample may have been obtained within 91 to 150 minutes after the first sample. In this case, the ACS classification algorithm to be applied in step e2) is a 0/2 hour algorithm. Alternatively, the second sample may have been obtained within 151 to 210 minutes after the first sample. In this case, the ACS classification algorithm to be applied in step e2) is a 0/3 hour algorithm.

The 0/1 hour, 0/2 hour and 0/3 hour ACS classification algorithms are known in the art. Preferred ACS classification algorithms are disclosed in Tables C1, C2 and C3.

Preferably, the method of the present invention excludes subjects which can be ruled in or ruled out already based on the amount of a cardiac Troponin in the first sample.

For example, the diagnosis of ACS can be ruled out if the subject has experienced the last episode of chest pain more than 3 hours ago and if the subject has an amount of a cardiac Troponin of below the limit of detection (LoD) in the first sample. Accordingly, the subject is not a subject who has experienced the last episode of chest pain more than 3 hours ago and if the subject has an amount of a cardiac Troponin of below the limit of detection (LoD) in the first sample. The LoD for cardiac Troponin T may be 5 ng/l. Thus, an amount of lower than 5 ng/l of a cardiac Troponin in the first sample is indicative for a rule out of ACS (in connection with a last chest pain episode more than 3 hours ago). The LoD for cardiac Troponin I may be 2 ng/l.

For example, the diagnosis of ACS can be ruled out, if the subject who has an amount of a cardiac Troponin which is indicative for an ACS in the first sample. Accordingly, the subject is not a subject who has an amount of a cardiac Troponin which is indicative for an ACS in the first sample. For example an amount of larger than 50 ng/l of a cardiac Troponin (such as TnT or TnI), such as an amount of larger than or equal to 52 ng/l, in the first sample is indicative for the rule in of ACS.

TABLE C1
Information on ACS classification algorithms (difference in times of tested, values
of troponin and output (Rule-out, Rule-in and observation zone), ESC recommended

| | Timing of last symptomatic | |
|---|---|---|
| | episode (if information | |
| Difference in times | available) and values of | |
| of troponin testing | troponin | Output |
| | last_symptom > 3 AND | Rule-out (ESC 0 h) |
| | hs-cTnT_c0 < 5 | Rule-in (ESC 0 h) |
| | hs-cTnT_c0 >= 52 | Rule-out (ESC 0 h) |
| | last_symptom > 6 AND | |
| | hs-cTnT_c0 <= 14 | |
| delta_t_min <= 30 | | Insufficient time |
| | | interval between |
| | | blood samples |
| delta_t_min > 30 AND | hs-cTnT_c0 < 12 AND | Rule-out (ESC 0/1 h) |
| delta_t_min <= 90 | ABSOLUTE(delta_c) < 3 | Observation zone (ESC 0/1 h) |
| | (hs-cTnt_c0 >= 12 OR | Rule-in (ESC 0/1 h) |
| | ABSOLUTE(delta_c) >= 3) AND | |
| | ABSOLUTE(delta_c) < 5 | |
| | ABSOLUTE(delta_c) >= 5 | |
| delta_t_min > 90 AND | hs-cTnt_c0 < 14 AND | Rule-out (0/2 h, |
| delta_t_min <= 150 | hs-cTnt_c1 < 14 AND | Reichlin 2015) |
| | ABSOLUTE(delta_c) < 4 | Observation zone |
| | (hs-cTnt_c0 >= 14 OR | (0/2 h, Reichlin 2015) |
| | hs-cTnt_c1 >= 14 OR | Rule-in (0/2 h, |
| | ABSOLUTE(delta_c) >= 4) AND | Reichlin 2015) |
| | (hs-cTnt_c0 < 53 OR | Rule-in (0/2 h, |
| | hs-cTnt_c1 < 53) AND | Reichlin 2015) |
| | ABSOLUTE(delta_c) < 10 | |
| | hs-cTnt c0 >= 53 AND | |
| | hs-cTnt_c1 >= 53 | |
| | ABSOLUTE(delta_c) >= 10 | |
| delta_t_min > 150 | hs-cTnt_c0 <= 14 AND | Rule-out (ESC 0/3 h) |
| | (hs-cTnt_c1 <= 14 OR | Rule-out (ESC 0/3 h) |
| | ABSOLUTE(delta_c) <= 7) | Rule-in (ESC 0/3 h) |
| | hs-cTnt_c0 > 14 AND | Rule-in (ESC 0/3 h) |
| | (hs-cTnt_c1 <= 14 OR | |
| | (ABSOLUTE(delta_c/ | |
| | hs-cTnt_c0)) <= 0.2) | |
| | hs-cTnt_c0 <= 14 AND | |
| | hs-cTnt_c1 > 14 AND | |
| | ABSOLUTE(delta_c) > 7 | |
| | hs-cTnt_c0 > 14 AND | |
| | hs-cTnt_c1 > 14 AND | |
| | (ABSOLUTE(delta_c/ | |
| | hs-cTnt_c0)) > 0.2 | |

TABLE C3
FDA recommended US protocol and the version without sex-specific
cutoffs for the 99th percentile upper limit of normal.

| | Timing of last symptomatic | |
|---|---|---|
| | episode (if information | |
| Difference in times | available) and values of | |
| of troponin testing | troponin | Output |
| | last_symptom > 3 AND | Rule-out (FDA 0 h) |
| | hs-cTnT_c0 < 6 | Rule-in (FDA 0 h) |
| | hs-cTnT_c0 >= 52 | Rule-out (FDA 0 h) |
| | last_symptom > 6 AND | |
| | hs-cTnT_c0 <= 19 | |
| delta_t_min <= 30 | | Insufficient time |
| | | interval between |
| | | blood samples |
| delta_t_min > 30 AND | hs-cTnT c0 < 12 AND | Rule-out (FDA 0/1 h) |
| delta_t_min <= 90 | ABSOLUTE(delta_c) < 3 | Observation zone |
| | (hs-cTnT_c0 >= 12 OR | (FDA 0/1 h) |
| | ABSOLUTE(delta_c) >= 3) AND | Rule-in (FDA 0/1 h) |
| | ABSOLUTE(delta_c) < 5 | |
| | ABSOLUTE(delta_c) >= 5 | |
| delta_t_min > 90 AND | hs-cTnT_c0 < 14 AND | Rule-out (0/2 h, |
| delta_t_min <= 150 | hs-cTnT_c1 < 14 AND | Reichlin 2015) |
| | ABSOLUTE(delta_c) < 4 | Observation zone |
| | (hs-cTnT_c0 >= 14 OR | (0/2 h, Reichlin 2015) |
| | hs-cTnT_c1 >= 14 OR | Rule-in (0/2 h, |
| | ABSOLUTE(delta_c) >= 4) AND | Reichlin 2015) |
| | (hs-cTnT_c0 < 53 OR | Rule-in (0/2 h, |
| | hs-cTnT_c1 < 53) AND | Reichlin 2015) |
| | ABSOLUTE(delta_c) < 10 | |
| | hs-cTnT_c0 >= 52 AND | |
| | hs-cTnT_c1 >= 52 | |
| | ABSOLUTE(delta_c) >= 10 | |
| delta_t_min > 150 | hs-cTnT_c0 <= 19 AND | Rule-out (FDA 0/3 h) |
| | (hs-cTnT_c1 <= 19 OR | Rule-out (FDA 0/3 h) |
| | ABSOLUTE(delta_c) <= 9) | Rule-in (FDA 0/3 h) |
| | hs-cTnT_c0 > 19 AND | Rule-in (FDA 0/3 h) |
| | (hs-cTnT_c1 <= 19 OR | |
| | (ABSOLUTE(delta_c/ | |
| | hs-cTnT_c0)) <= 0.2) | |
| | hs-cTnt_c0 <= 19 AND | |
| | hs-cTnt_c1 > 19 AND | |
| | ABSOLUTE(delta_c) > 9 | |
| | hs-cTnt_c0 > 14 AND | |
| | hs-cTnt_c1 > 19 AND | |
| | (ABSOLUTE(delta_c/ | |
| | hs-cTnt_c0)) > 0.2 | |

Table C2 shows the specific modifications of diagnostic protocols recommended by 2020 ESC Guidelines including ESC 0/1, ESC 0/2 and ESC 0/3 protocol when FDA recommendations are considered. This example refers to the use of the Roche hs-cTnT assay but is assay specific for hs-cTnI assays. In this table the single-99^{th} percentile cutoff of 19 ng/L is applied instead of the sex-specific hs-cTnT 99th percentile cutoffs for males (22 ng/L) and females (14 ng/L). In addition, the lower reporting limit proposed by the FDA (6 ng/L) has been applied.

TABLE C3
FDA recommended US protocol and the version with sex-specific
cutoffs for the 99^{th} percentile upper limit of normal.

| | Timing of last symptomatic | |
|---|---|---|
| | episode (if information | |
| Difference in times | available) and values of | |
| of troponin testing | troponin | Output |
| | last_symptom > 3 AND | Rule-out (FDA 0 h) |
| | hs-cTnT_c0 < 6 | Rule-in (FDA 0 h) |
| | hs-cTnT_c0 >= 52 | Rule-out (FDA 0 h) |
| | last_symptom > 6 AND | |
| | hs-cTnT_co <= 14 for | |
| | females and 22 for males | |
| delta_t_min <= 30 | | Insufficient time |
| | | interval between |
| | | blood samples |
| delta_t_min > 30 AND | hs-cTnT_c0 < 12 AND | Rule-out (FDA 0/1 h) |
| delta_t_min <= 90 | ABSOLUTE(delta_c) < 3 | |
| | (hs-cTnt_c0 >= 12 OR | Observation zone |
| | ABSOLUTE(delta_c) >= 3) AND | (FDA 0/1 h) |
| | ABSOLUTE(delta_c) < 5 | Rule-in (FDA 0/1 h) |
| | ABSOLUTE(delta_c) >= 5 | |
| delta_t_min > 90 AND | hs-cTnT_c0 < 14 AND | Rule-out (0/2 h, |
| delta_t_min <= 150 | hs-cTnT_cl < 14 AND | Reichlin 2015) |
| | ABSOLUTE(delta_c) < 4 | Observation zone |
| | (hs-cTnT_c0 >= 14 OR | (0/2 h, Reichlin 2015) |
| | hs-cTnT_c1 >= 14 OR | Rule-in (0/2 h, |
| | ABSOLUTE(delta_c) >= 4) AND | Reichlin 2015) |
| | (hs-cTnT_c0 < 52 OR | Rule-in (0/2 h, |
| | hs-cTnT_c1 < 52) AND | Reichlin 2015) |
| | ABSOLUTE(delta_c) < 10 | |
| | hs-cTnT_c0 >= 52 AND | |
| | hs-cTnT_c1 >= 52 | |
| | ABSOLUTE(delta_c) >= 10 | |
| delta_t_min > 150 | hs-cTnT_c0 <= 14 for females | Rule-out (FDA 0/3 h) |
| | or 22 for males AND | Rule-out (FDA 0/3 h) |
| | (hs-cTnT_c1 <= 14 for females | Rule-in (FDA 0/3 h) |
| | or 22 for males OR | Rule-in (FDA 0/3 h) |
| | ABSOLUTE(delta_c) <= 7 for | |
| | females or 11 for males) | |
| | hs-cTnT_c0 > 14 for females | |
| | or 22 for males AND | |
| | (hs-cTnT_c1 <= 14 for | |
| | females or 22 for males OR | |
| | (ABSOLUTE(delta_c/ | |
| | hs-cTnT_c0)) <= 0.2) | |
| | hs-cTnT_c0 <= 14 for females | |
| | or 22 for males AND | |
| | hs-cTnT_c1 > 14 for females | |
| | or 22 for males AND | |
| | ABSOLUTE(delta_c) > 7 for | |
| | females or > 11 for males | |
| | hs-cTnT_c0 > 14 AND | |
| | hs-cTnT_c1 > 19 AND | |
| | (ABSOLUTE(delta_c/ | |
| | hs-cTnT_c0)) > 0.2 | |

Table C3 shows the specific modifications of diagnostic protocols recommended by 2020 ESC Guidelines including ESC 0/1, ESC 0/2 and ESC 0/3 protocol when FDA recommendations are considered. This example refers to the use of the Roche hs-cTnT assay but is assay specific for hs-cTnI assays. In this table sex-specific hs-cTnT 99^{th} percentile cutoffs for males (22 ng/L) and females (14 ng/L) are used instead of the single-cutoff of 19 ng/L. In addition, the lower reporting limit proposed by the FDA (6 ng/L) has been applied.

The result of the assessment made in the methods of the present invention, such as the score or information on the classification may be displayed on a display. Alternatively, or additionally, the result may be printed by a printer.

In an embodiment of the methods of the present invention, the methods may comprise the further step of transferring the result obtained of the method of the present invention to the is individual's electronic medical records.

In an embodiment of the present invention, the method of the present invention further encompasses the recommendation or initiation of a suitable treatment for the subject, once the subject has been classified (for example, if the patient is ruled int to suffer from MI). Suitable treatments are known in the art, and e.g. described in the ESC guidelines which herewith are incorporated by reference with respect to the entire disclosure content (Roffi M, Patrono C, Collet J P, Mueller C, Valgimigli M, Andreotti F, Bax J J, Borger M A, Brotons C, Chew D P, Gencer B, Hasenfuss G, Kjeldsen K, Lancellotti P, Landmesser U, Mehilli J, Mukherjee D, Storey R F, Windecker S; ESC Scientific Document Group. 2015 ESC Guidelines for the management of acute coronary syndromes in patients presenting without persistent ST-segment elevation: Task Force for the Management of Acute Coronary Syndromes in Patients Presenting without Persistent ST-Segment Elevation of the European Society of Cardiology (ESC). Eur Heart J. 2016 Jan. 14; 37(3):267-315).

The definitions given herein above preferably apply mutatis mutandis to the following: The present invention further contemplates a method for patient management in an emergency department having a plurality of patients with suspected ACS per day, said method comprising carrying out for each of said patients steps a) to f) of the classification method, thereby identifying patients
i) in which the diagnosis of myocardial infarction can be ruled in (rule-in),
ii) in which the diagnosis of myocardial infarction can be ruled out (rule-out), and
iii) who require further examination for ruling in or ruling out myocardial infarction (observation zone).

The present invention further contemplates a method for increasing the proportion of patients with a rule-out diagnosis of myocardial infarction within a plurality of patients with suspected ACS, wherein said method comprises carrying out steps a) to f) of the classification method for said plurality of patients.

The present invention further relates to a computer program including computer-executable instructions for performing the steps of the computer-implemented method according to the present invention, when the program is executed on a computer or computer network. Typically, the computer program specifically may contain computer-executable instructions for performing the steps of the method as disclosed herein. Specifically, the computer program may be stored on a computer-readable data carrier.

The present invention further relates to a computer program product with program code means stored on a machine-readable carrier, in order to perform the method according to the present invention, when the program is executed on a computer or computer network, such as one or more of the above-mentioned steps discussed in the context of the computer program. As used herein, a computer program product refers to the program as a tradable product. The product may generally exist in an arbitrary format, such as in a paper format, or on a computer-readable data carrier. Specifically, the computer program product may be distributed over a data network.

The present invention further relates to a computer or computer network comprising at least one processing unit, wherein the processing unit is adapted to perform all steps of the method according to the present invention, in particular steps a), b), c) and d).

The present invention also, in principle, contemplates a computer program, computer program product or computer readable storage medium having tangibly embedded said computer program, wherein the computer program comprises instructions when run on a data processing device or computer carry out the method of the present invention as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a data for of parameters as defined herein elsewhere, and
a data stream signal, typically encrypted, comprising the score calculated by the methods of the present invention and, preferably, providing information of the classification.

The present invention further relates to a device for classifying a patient with suspected acute coronary syndrome, said device comprising a processing unit, and a computer program including computer-executable instructions (such as a computer program as set forth above), wherein said instructions, when executed by the processing unit, cause the processing unit to perform the computer-implemented method according to the present invention, i.e. to perform the steps of said method. The device may further comprise a user interface and a display, wherein the processing unit is coupled to the user interface and the display. Typically, the device provides as output the classification. In an embodiment, the classification is provided on the display. Typically, the device comprises software being tangibly embedded into said device and, when running on said device, carries out the method of the present invention.

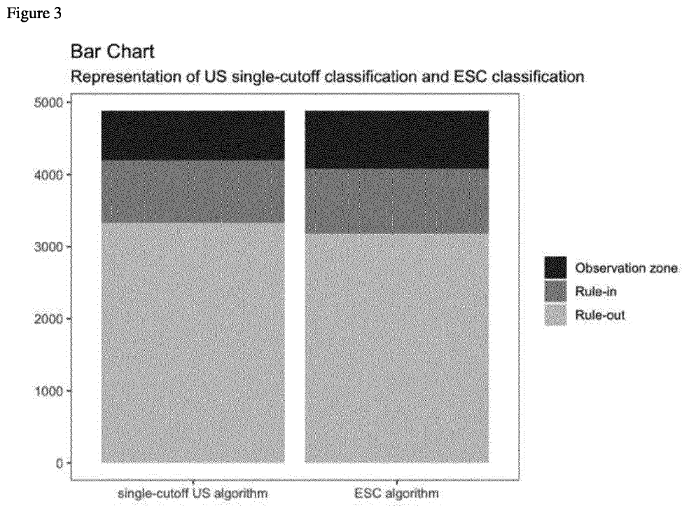

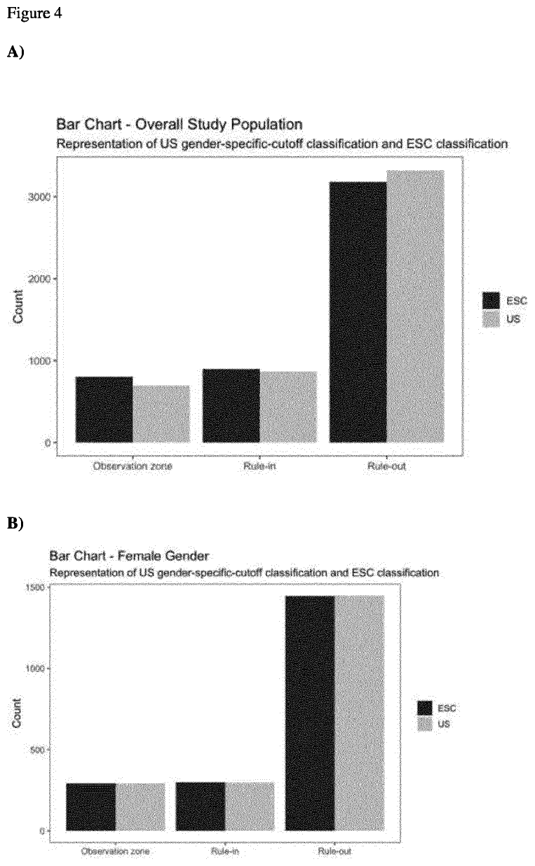

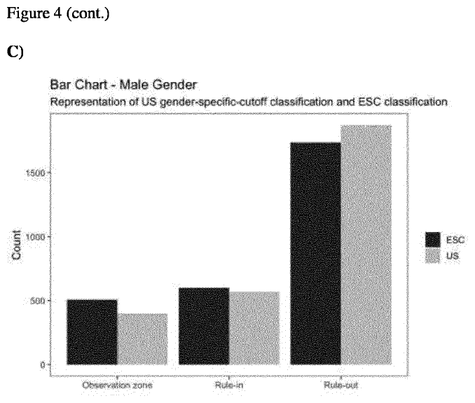

In the following, the advantages of the classification method of the present invention as compared to standard classification are summarized.
1. Interactive guidance of ED physician through the triage process of patients presenting with suspected ACS
2. Indication of the need and proposal of the optimal time point (including a tolerance time of 29 minutes) for a second or third blood sample following 2020 ESC Guideline recommendations.
3. Interpretation of blood results (cardiac troponin levels) in accordance with truly elapsed time intervals between the first and the second blood sample instead of a 0/1 hour protocol by default (FIGS. 2A and B and table in Example 4)
4. Indication of the need and proposal of the correct time point for a third blood draw if the second blood draw is not diagnostic, i.e. neither rule-out nor rule-in.
5. Selection of a country-specific classification, e.g. FDA recommended version with or without consideration of sex-specific 99^{th} percentile upper limits of normal. The use of a country specific classification results into a relevant number of re-classifications compared to the non-US version, with minor effect of the use of a higher lower reporting limit (6 ng/L versus 5 ng/L), the use of a higher single 99^{th} percentile cutoff (19 ng/L versus 14 ng/L), or the use of a sex-specific cutoff for females versus males (14 ng/L versus 22 ng/L) (FIGS. 3 and 4).

In summary, the present invention allows for a more accurate classification of patients with suspected ACS (FIGS. 2A and B). A substantial proportion of patients is re-classified when truly elapsed intervals between blood draws are considered. In addition, the “observational zone” is dissolved improving patient disposition by re-classification into either “rule-out” or “rule-in” after a third blood draw collected 3 hours or later after the first blood draw. Advantageously, protocol adherence and protocol deviations at all critical time-points with the potential to increase transparency of the diagnostic process guideline-adherence and quality of care. For example, a missing second blood draw (i.e. second sample) if time of chest pain onset is unknown or below 3 hours (see Table in Example 4) can be indicated, thereby improving protocol-adherence and increasing safety of early discharge, if discharge is based on the classification of rule-out based on a single very low high sensitivity cardiac troponin. Further, cost effectiveness can be increased by reducing the numbers of unnecessary additional blood draws beyond a diagnostic set, i.e. a second blood draw after a diagnostic 0-hour sample, or a third blood draw after a diagnostic second blood draw (see table in Example 4). Cost effectiveness is plausible because earlier diagnostic classification without unnecessary blood draws is likely to reduce staff time for monitoring in the ED, reduced time for blood collection, lower laboratory costs. Further, the method of the present invention provides the option to adapt the protocols if Guideline recommendation differ or change.

### EMBODIMENTS OF THE PRESENT INVENTION

In the following, embodiments of the present invention are disclosed. The definitions given is herein above apply mutatis mutandis to the following.
1. A computer-implemented method for classifying a patient with suspected acute coronary syndrome, comprising the steps of
   (a) receiving, at a processing unit, information on a first time-point at which a first sample has been obtained from the patient at presentation,
   (b) providing on a display
      b1) a proposal for a second time-point at which a second sample shall be obtained from the patient, wherein the second time point is within an interval of about 1 hour or of about 2 hours after the first time point, and
      b2) a proposal for an ACS classification algorithm to be applied for the classification of the patient, wherein the ACS classification algorithm is based on the second time point of proposed in step b1)
   (c) receiving, at a processing unit:
      c1) information on the actual time-point at which the second sample has been obtained,
      c2) a value for the amount of the cardiac Troponin in the first sample, and
      c3) a value for the amount of the cardiac Troponin in the second sample
   (d) carrying out by the processing unit an analysis on whether the second sample has been obtained within the interval under b1), wherein the sample is considered to have been obtained within the interval of about 1 hour, if it has been obtained between 30 to 90 minutes after the first sample, and/or wherein the second sample is considered to have been obtained within the interval of about 2 hours, if it has been obtained between 91 to 150 minutes after the first sample, and
   (e) classifying the patient by the processing unit, wherein
      e1) the patient is classified based on the ACS classification algorithm proposed in step b2), if the second sample has been obtained within the interval proposed in step b1), or
      e2) wherein the patient is classified by an ACS classification algorithm which differs from the ACS classification algorithm proposed in step b2), if the sample has not been obtained within the interval proposed in step b1), and, optionally,
   (f) providing information on the classification of the patient on a display.
2. The method of embodiment 1, wherein the sample is a blood, serum or plasma sample. 3. The method of embodiments 1 and 2, wherein the second time-point proposed in step
b1) is within an interval of about one hour after the first sample, and wherein the ACS classification algorithm proposed in step b2) is a 0/1 hour algorithm.
4. The method of embodiments 1 to 3, wherein the ACS classification algorithm to be applied in step e2) is a 0/2 hour algorithm, if the second sample has been obtained within 91 to 150 minutes after the first sample.
5. The method of embodiment 1 to 4, wherein the ACS classification algorithm to be applied in step e2) is a 0/3 hour algorithm, if the second sample has been obtained within 151 to 210 minutes after the first sample.
6. The method of any one of embodiments 1 to 5, wherein step b) further comprises providing, on the display, information on whether a second sample is necessary or not.
7. The method of any one of embodiments 1 to 6, wherein the proposal under b1) and b2) is made in case that the patient cannot be classified based on the first sample only.
8. The method of any one of embodiments 1 to 7, wherein the patient is classified as suffering from acute coronary syndrome (“rule in”), as not suffering from acute coronary syndrome (“rule out”), or as requiring further classification in order to rule in or rule out myocardial infarction (“observe zone” or “observation zone”).
9. The method of any one of embodiments 1 to 8, wherein step f) further comprises providing on the display information on whether a third sample is necessary for further classification of the patient.
10. The method of embodiment 9, wherein a third sample is necessary, if the patient is classified into the “observational zone” after the second sample.
11. The method of embodiment 10, wherein step f) further comprises, providing, on the display, a proposal for a third time point at which a third sample shall be obtained from the patient, wherein the third time point is 3 hours or later after the first sample.
12. A method for patient management in an emergency department having a plurality of patients with suspected ACS per day, said method comprising carrying out for each of said patients steps a) to f) of the method of embodiment 1 1, thereby identifying patients
   i) in which the diagnosis of myocardial infarction can be ruled in (rule-in),
   ii) in which the diagnosis of myocardial infarction can be ruled out (rule-out), and
   iii) who require further examination for ruling in or ruling out myocardial infarction (observation zone).
13. A method for increasing the proportion of patients with a rule-out diagnosis of myocardial infarction within a plurality of patients with suspected ACS, wherein said method comprises carrying out steps a) to f) of embodiment 1 1 for said plurality of patients.
14. The method of any one of embodiments 1 to 13, wherein the subject is a human subject.
15. The method of any one of embodiments 1 to 14, wherein the cardiac Troponin is cardiac Troponin T.
16. The method of any one of embodiments 1 to 14, wherein the cardiac Troponin is cardiac Troponin I.
17. A device for classifying a patient with suspected acute coronary syndrome, said device comprising a processing unit, and a computer program including computer-executable instructions, wherein said instructions, when executed by the processing unit, cause the processing unit to perform the computer-implemented method according to any one of embodiments 1 to 10.

### THE FIGURES SHOW

FIG. 1 A) Sampling interval adjusted classification tool for patients with suspected acute coronary syndrome. The method of the present invention allows for the implementation of different diagnostic protocols, such as the FDA or ESC diagnostic protocols. Further, different cutoffs can be implemented, such as sex-specific cutoffs versus a single sex-independent cutoff in the US.
B) Detailed depiction for a sampling interval adjusted Classification Tool for Patients with suspected Acute Coronary Syndrome (LoD: Limit of detection, hsTn: high sensitive cardiac Troponin, such as Troponin I or T, hscTnT: high sensitive cardiac Troponin T, ULN: Upper limit of normal.

FIG. 2 A) Effect of time delays on classification rules of patients with suspected NSTE-ACS. The time delay for the scheduled rule is shown on the x-axis. A delay of >30 minutes, i.e. >90 min interval between admission sample and follow-up sample would start to the eligibility for a 0/2 hour protocol rather than a 0/1 hour protocol to adjust time-dependent increases of troponin. Accordingly, rates of patients categorized as rule-out start to decline. As a consequence, an incorrectly lower rate of ruled-out patients would decrease the opportunity to discharge a substantial proportion of low-risk patients, and would increase unwarranted treatments and unnecessary invasive procedures in patients assigned falsely to a rule-in or observe zone category. The proposed tool integrates the calculation of rules based on their validated protocols but also proposes optimal time points with corresponding upper and lower intervals for the next blood draw, and provides correct interpretation of findings based on the actual protocol and not on primarily intended protocol. Thus, the tool provides a step-up or step-down of an intended protocol, e.g. from a 0/1 hour to an ESC or ADP 0/2 hour or vice versa. The tool is not limited to currently established protocols but can be updated to implement upcoming protocols.
B) Classification of suspected ACS with and without consideration of truly elapsed time interval between first and second blood draw. The figure illustrates the change of classification of patients with suspected ACS when the truly elapsed time between blood samples is considered. The calculations in this example are based on the use of the Roche hs-cTnT assay.
In the left panel, it is assumed that physicians use the 0/1 hour algorithm by default. However, cardiac troponin concentrations in peripheral blood increase with increasing time after the ischemic index event. Therefore, cutoff values and concentration changes are progressively higher in the 0/2 hour algorithm and the 0/3 hour algorithms. Not considering the truly elapsed time between the first and second blood draw bears the risk for misclassification. The panel demonstrates that adjustment of time increases the proportion of patients classified as rule-out and decreases the proportion of patients classified as rule-in or the observational zone.

FIG. 3 Classification of suspected ACS based on FDA recommended algorithm with single hs-cTnT cutoff at 19 ng/L versus ESC algorithm using 14 ng/L as 99th percentile cutoff.
The figure illustrates the change of classification of patients with suspected ACS when the FDA version using FDA recommended reporting limits and a single-sex independent 99th percentile upper limit of normal versus the ESC recommended algorithms. Both calculations have been made after adjustment for truly elapsed time intervals between the first and the second blood draw. The calculations in this example are based on the FDA recommendations on cutoffs and lower reporting limits when the Roche hs-cTnT assay is used.
The panel demonstrates that application of the FDA recommended modifications increases the proportion of patients classified as rule-out and slightly decreases the proportion of patients classified as rule-in or the observational zone.

FIG. 4 Classification of suspected ACS based on FDA recommended algorithm applying sex-specific cutoffs—14 ng/L for females and 22 ng/L for males. A) overall cohort (females and males), B) Female gender C) male gender.
The figures illustrates the change of classification of patients with suspected ACS when FDA recommended version with use of sex-specific 99^{th} percentile upper limit of normal is applied versus the ESC recommended algorithms. Both calculations have been made after adjustment for truly elapsed time intervals between the first and the second blood draw. The calculations in this example are based on the FDA recommendations on sex-specific cutoffs and lower reporting limits when the Roche hs-cTnT assay is used.

The panel demonstrates that application of FDA recommended sex-specific cutoffs almost exclusively affect the reclassification in men but not in women. In men the proportion of patients classified as rule-out using the FDA version increases compared to the ESC version whereas the proportion of patients classified as rule-in or into the observational zone slightly decreases.

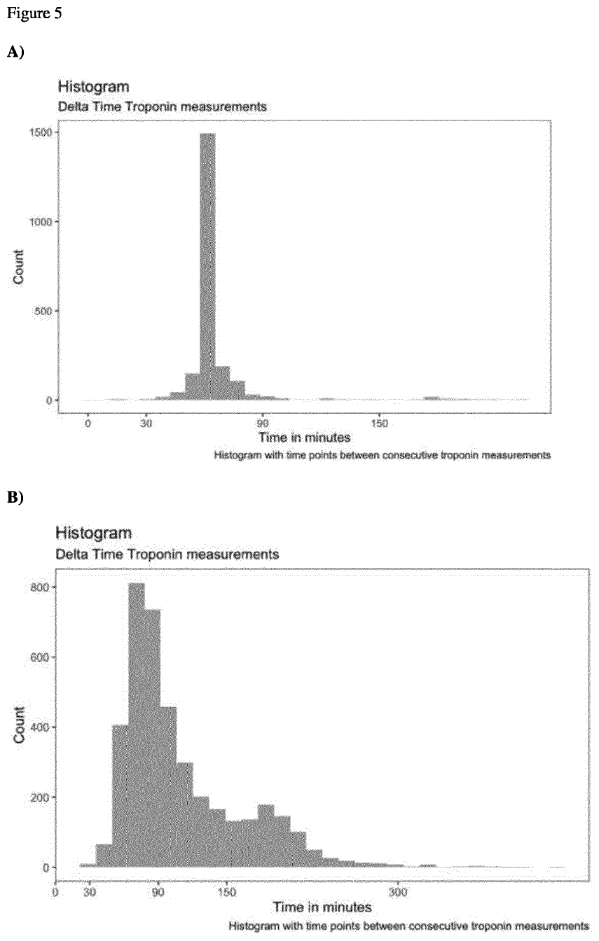

FIG. 5 A) The “ideal world” with management of patients with suspected ACs under conditions that are typical for controlled clinical trials. The figure shows findings from an observational trial on 2,146 patients where the second blood draw was timed exactly 60 minutes after the first blood draw using a stopwatch.
B) The “real world evidence” in an ED showing heterogenous, right-skewed distribution of time intervals between the first and second blood draw, with a majority of second blood draws beyond a 60 minute time frame ±30 minute tolerance interval.

### EXAMPLES

The Examples shall illustrate the invention. They shall be no means construed as limiting the scope.

### Example 1: Cohorts

The derivation and validation cohort are independent and sequential without overlap of recruitment period or patients. A recruitment period of 12 month eliminates a bias related to calendar dependent seasonal differences of prevalent diagnoses and reduces the potential effect of different crowding levels on the management of patients. Although study populations were enrolled in a single emergency department, standardized diagnostic protocols were used and the adherence to the most recent ESC guideline recommendations ((Roffi et al. Eur Heart J 2016; 37(3):267-315; Amsterdam et al., Am Coll Cardiol 2014; 64(24):e139-e228) on non-ST-elevation acute coronary syndrome (NSTE-ACS) and differential diagnoses is being regularly audited by the DGK (Deutsche Gesellschaft fir Kardiologie, German Society of Cardiology).

In the derivation and validation cohorts, patients with suspected ACS were enrolled based on a broad spectrum of presenting symptoms including dyspnea, gastric discomfort, back or shoulder pain, isolated radiating arm pain, diaphoresis, thus enabling a broader extrapolation of the algorithms to less selected patients. Oppositely, enrolment based on typicality of chest pain symptoms would have an overoptimistic yield of the machine learned algorithm, since the performance of a test depends on the pre-test probability. Patients were enrolled continuously to minimize a selection bias. Patients were excluded retrospectively in the presence of pre-specified exclusion criteria including presence of persisting ST-segment elevation or new left bundle branch block, missing laboratory values for the initial hs-cTnT or on the complete set of a diagnostic pair of troponin results, referral from other hospitals for dedicated services, e.g. coronary angiography or reperfusion therapies obviating a full diagnostic protocol. In addition, patients were retrospectively removed from the analysis if they had any of the following conditions:
Age<18 years
End-stage renal disease (ESRD)
Acute decompensated heart failure in the absence of signs or symptoms of an ACS, and low pre-test probability of underlying ischemic heart disease
New atrial tachyarrhythmias (atrial flutter, atrial fibrillation, atrial tachycardia, atrioventricular nodal re-entry tachycardia) in the absence of signs or symptoms of an ACS, and low pre-test probability of underlying ischemic heart disease
Ventricular tachyarrhythmias or ICD shock in the absence of signs or symptoms of an ACS
Patients with very high pre-test probability for a differential diagnosis such as presentation with fever suggesting pneumonia or pleutitic pain, swollen or painful lower extremities suggesting venous thromboembolism (VTE), clinical suspicion of pneumothorax
Patients presenting with active cancer and life expectancy <1 year AND absence of signs or symptoms of ACS
Patients with end-stage cancer and expected life expectancy <6 months
a Patients that were resuscitated out of hospital

To avoid overfitting, resulting in overoptimistic prediction models, all ML algorithms were generated in a derivation cohort and were validated in an independent representative validation cohort enrolled during an equally long recruitment period, thus taking into account seasonal changes of patient volumes, disease representation, and crowding.

### Example 2: Development of Classification Tool

In the studies underlying the present invention, electronic support for the classification of a patient presenting with suspected ACS or a differential diagnosis of ACS was established. The classification algorithm requires imputation of paired high-sensitivity cardiac troponin T concentrations and concentration change of cardiac troponin T concentrations in the second blood draw, and time from onset of symptoms to the initial blood draw. Based on the published literature and supported by ESC guideline recommendations, classification is reported for ESC 0/1 hour, ESC 0/2 hour, ADP 2 hour, ESC 0/3 hour, ESC 0/6-9 hour protocols and is accompanied by the classification together with the corresponding reference. The diagnostic rules are calculated for time intervals between blood sampling, i.e. within 30-90 minutes after the first blood draw for ESC 0/1 hour protocol, 91-150 minutes for ESC 0/2 hour protocol, 151-210 minutes for ESC 0/3 hour protocol, and >210 minutes for the ESC 0/6-9 hour protocol. The ESC 0/3 hour and 0/6-9 hour protocols are based on the Universal MI definition and the 99^{th} percentile value. In the ESC 0/3 hour protocol a relevant concentration change is defined as a rise of hs-cTnT of 50% of the upper limit of normal if the initial hs-cTnT is below the 99′ percentile, and by a rise and/or fall by 20% from the baseline value if the initial hs-cTnT is above the 99^{th} percentile. The classification includes the ESC 0 hour protocol, a protocol that allows rule-out an MI with a single hs-cTnT value below the LoD (5 ng/L) at presentation, provided the patient presents more than 3 hours after onset of symptoms, or rule-in based on a very high hs-cTnT concentration above 52 ng/L at presentation.

The aetiology of acute coronary syndromes (ACS) is complex and involves multiple interrelated mechanisms, of which many have yet not been fully understood. Our current understanding is that a plaque may rupture or erode, in response to inflammation, leading is to local occlusive or non-occlusive thrombosis (Braunwald, Circulation 1998; 98(21):2219-22). Depending on the degree and reversibility of this dynamic obstruction, the clinical manifestations of ACS comprise a continuous spectrum of risk that progresses from unstable angina (UA) to non-ST-segment elevation myocardial infarction (NSTEMI) to ST-segment elevation myocardial infarction (STEMI). NSTEMI is distinguished from UA by ischemia sufficiently severe in intensity and duration to cause myocyte necrosis, which is recognized by the detection of cardiac troponin (cTn), the most sensitive and specific biomarker of myocardial injury. cTnT and cTnI are now considered as the preferred biomarkers for the diagnosis of myocardial injury, as the cardiac isoforms of troponin T or I are expressed exclusively in myocytes on the thin myofilament of the contractile apparatus and, to a lower degree (3-6%), as unbound proteins in the cytoplasm of myocytes.

The most recent achievement with biomarker testing is the implementation of high-sensitivity troponin (hs-cTn) assays, instead of the conventional, less sensitive troponin assays, in patients with suspected ACS (Giannitsis et al., Clin Chem 2010; 56(2):254-61). The term “high-sensitivity cardiac troponin T (hs-cTnT, hsTnT, cTnThs) indicates a generation of cTnT (5^{th} generation or higher) characterized by improved analytical sensitivity and unchanged tissue specificity compared to the processor assays. A high-sensitivity designation is fulfilled according to the criteria of the Academy of the American Association for Clinical Chemistry (AACC) and the International Federation of Clinical Chemistry and Laboratory Medicine (IFCC), when an assay is able to measure cTn concentrations precisely at or below the 99th percentile value and by an analytical sensitivity defined by a percentage of 50% or more detectable values above the Limit of Detection (LoD) in a healthy reference population in both genders (Wu et al., Clin Chem 2018; 64(4):645-655). The invention is based on the use of a hs-cTn assay but is not restricted to the list of assays currently designated as high sensitivity assays but is inclusive of assays that are being added to the IFCC list.

According to the Universal MI definition (Thygesen et al. Eur Heart J 2019; 40(3):237-269), a diagnosis of an MI is made when cTn is elevated above the upper reference limit of a healthy reference population indicating the presence of myocardial injury, together with a relevant acute concentration change (rise and/or fall) of cTn indicating the acute event and is the presence of a clinical sign or symptom that indicated an underlying context of myocardial ischemia. These features should include at least one of the following: symptoms of ischemia, new or presumed new significant ST-segment-T wave (ST-T) changes or new left bundle branch block, development of pathological Q waves in the ECG, imaging evidence of new loss of viable myocardium or new regional wall motion abnormality, evidence of intracoronary thrombus by angiography or autopsy. Along with the general definition of MI, five subtypes of MI have been defined by the 4^{th} version of the UDMI of which type 1-3 MI are related to spontaneous MI and type 4-5 define procedure-related MIs (Thygesen et al. Eur Heart J 2019; 40(3):237-269).

In the present derivation and validation studies, the diagnosis of NSTEMI was not further subclassified into type 1 or type 2 MI, or other subtypes.

During the entire enrolment process, patients were categorized into rule-out, observational zone or rule-in, and hs-cTnT was used routinely applying recommended thresholds and concentration changes.

UA was diagnosed in the presence of symptoms suggestive of myocardial ischemia together with biomarker results that do not fulfill the criteria of MI according to the UDMI. Variations in the rates of UA are most likely explained by the lack of a universally accepted definition of UA. In most definitions (Roffi et al. Eur Heart J 2016; 37(3):267-315; Amsterdam et al., Am Coll Cardiol 2014; 64(24):e139-e228), the key characteristic is the absence of myocardial injury as indicated by serial hs-cTn (Sandoval et al. Eur Heart J Acute Cardiovasc Care 2018; 7(2):120-128). However, the use of hs-cTn assays will ultimately identify some patients classified clinically as UA due to unstable chest discomfort with angina at rest, or new onset or worsening of angina who have stable (without a rise and/or fall) elevations of hs-cTn values above the 99a percentile (Braunwald, Circulation 2013; 127(24):2452-7). These elevations are stable in serial samples reflecting end-stage renal disease or underlying structural heart disease or coronary artery disease^{31, 40}. This scenario is an important issue that has been ignored in the literature, as highlighted by the International Federation of Clinical Chemistry (IFCC) in their 2015 educational document regarding hs-cTn assays (Apple et al., Clin Biochem 2015; 48(4-5):201-3). The symptoms in UA are explained by obstructive coronary artery disease (CAD) or vasospasm, presumably without plaque rupture or activation of coagulation, as suggested by the lack of benefit from anticoagulants (Morrow et al., J Am Coll Cardiol 2000; 36(6):1812-7) or antiplatelet therapies (see e.g. Wallentin et al., Circulation 2014; 129(3):293-303, and even early revascularization in patients without elevated cTn. In general, outcomes of patients presenting with UA are regarded substantially more benign than among patients with NSTEMI Accordingly, the 2015 ESC guidelines on the management of patients with non-ST-segment elevation acute coronary syndrome (NSTE-ACS) discourage routine coronary angiography and recommend a selective-invasive strategy for those who continue to experience symptoms despite an optimal medical treatment, or for those with objective evidence for inducible myocardial ischemia. Guidelines also recommend discharging low risk patients after individual risk stratification. The fear of missing an impending MI, however, results in a liberal referral practice of patients with presumed UA to acute coronary angiography. Registries demonstrate that patients with UA are frequently admitted to hospitals, and receive unnecessary coronary angiographies

STEMI is defined in the presence of persisting ST segment elevations in at least 2 contiguous leads or a new bundle branch block (right or left bundle branch block) or a permanently paced rhythm.

A faster protocol with testing of hs-cTn at presentation and after 3 hours is recommended by ESC guidelines, ACC/AHA and other International Guidelines when hs-cTn assays are used. Faster protocols and accelerated diagnostic protocols (necessitating the use of a clinical scoring system) with hs-cTn testing at presentation and within 120 minutes are a being recommended by ESC guidelines (Eur Heart J 2016; 37(3):267-315) and APAC guidelines (Circ J 2020; 84(2):136-143) when a hs-cTn assay that has been validated for this purpose is available. Forhs-cTnT, the classification of rule-in and rule-out for the ESC 0/3 hour protocol and for the three level classification when faster protocols are used is listed in the following Table.

TABLE
Classification

| | Timing of last symptomatic | |
|---|---|---|
| | episode (if information | |
| Difference in times | available) and values of | |
| of troponin testing | troponin | Output |
| | last_symptom > 3 AND | Rule-out (ESC 0 h) |
| | hs-cTnT_c0 < 5 | Rule-in (ESC 0 h) |
| | hs-cTnT c0 >= 52 | Rule-out (ESC 0 h) |
| | last_symptom > 6 AND | |
| | hs-cTnT_c0 <= 14 | |
| delta_t_min <= 30 | | Insufficient time |
| | | interval between |
| | | blood samples |
| delta_t_min > 30 AND | hs-cTnT_c0 < 12 AND | Rule-out (ESC 0/1 h) |
| delta_t_min <= 90 | ABSOLUTE(delta_c) < 3 | Observation zone |
| | (hs-cTnT_c0 >= 12 OR | (ESC 0/1 h) |
| | ABSOLUTE(delta_c) >= 3) AND | Rule-in (ESC 0/1 h) |
| | ABSOLUTE(delta_c) < 5 | |
| | ABSOLUTE(delta_c) >= 5 | |
| delta t_min > 90 AND | hs-cTnT_c0 < 14 AND | Rule-out (0/2 h, |
| delta_t_min <= 150 | hs-cTnT_c1 < 14 AND | Reichlin 2015) |
| | ABSOLUTE(delta_c) < 4 | Observation zone |
| | (hs-cTnT_c0 >= 14 OR | (0/2 h, Reichlin 2015) |
| | hs-cTnT_c1 >= 14 OR | Rule-in (0/2 h, |
| | ABSOLUTE(delta_c) >= 4) AND | Reichlin 2015) |
| | (hs-cTnT_c0 < 52 OR | Rule-in (0/2 h, |
| | hs-cTnT_c1 < 52) AND | Reichlin 2015) |
| | ABSOLUTE(delta_c) < 10 | |
| | hs-cTnT c0 >= 52 AND | |
| | hs-cTnT_c1 >= 52 | |
| | ABSOLUTE(delta_c) >= 10 | |
| delta_t_min > 150 | hs-cTnT_c0 <= 14 AND | Rule-out (ESC 0/3 h) |
| | (hs-cTnT_c1 <= 14 OR | Rule-out (ESC 0/3 h) |
| | ABSOLUTE(delta_c) <= 7) | Rule-in (ESC 0/3 h) |
| | hs-cTnT_c0 > 14 AND | Rule-in (ESC 0/3 h) |
| | (hs-cTnT_c1 <= 14 OR | |
| | (ABSOLUTE(delta_c/ | |
| | hs-cTnT_c0)) <= 0.2) | |
| | hs-cTnT_c0 <= 14 AND | |
| | hs-cTnT_c1 > 14 AND | |
| | ABSOLUTE(delta_c) > 7 | |
| | hs-cTnT_c0 > 14 AND | |
| | hs-cTnT_c1 > 14 AND | |
| | (ABSOLUTE(delta_c/ | |
| | hs-cTnT_c0)) > 0.2 | |

The corresponding classification with the US protocol and the subcategories with/or without sex-specific cutoffs for the 99th percentile upper limit of normal are shown in Tables C2 and C3, respectively (see above).

### Myocardial Infarction

MI was diagnosed according to the criteria of the 3^{rd} universal definition of myocardial infarction. Adjudication of MI at follow-up was left at the discretion of the attending physician if the event occurred outside the study site. Otherwise, adjudication was done by two cardiologists and in case of disagreement by a third cardiologist based on all available clinical information. Myocardial infarcts were specified as type 1, type 2, type 4a-c or type but were—at the end—summarized as any non-fatal MI.

The ML algorithm was constructed to enable an accurate, reproducible and evidence-based classification of patients presenting with suspected ACS into a rule-out zone, an observe zone, and a rule-in zone.

### Example 3: Diagnostic Re-Classification

The following table illustrates that reclassification after adjustment of time may lead to reclassification from the observational zone to rule-out and less often to rule-in. In addition, patients may be reclassified from rule-in to the observational zone and to rule-out. No reclassifications occurred in the rule-out group. Of note, the calculations in this example are based on the use of the Roche hs-cTnT assay.

TABLE
Diagnostic re-classification table of the 0/1 hour protocol with and
without consideration of truly elapsed time between blood draws

| | Time interval adjusted | | |
|---|---|---|---|
| | Observation zone | Rule-in | Rule out |
| Observation zone | 728 | 5 | 331 |
| Rule-in | 72 | 892 | 44 |
| Rule-out | 0 | 0 | 2807 |

### Example 4: Protocol Adherence (Avoiding of Missing and Excessive Troponin Measurements

Protocol adherence regarding the correct number of required blood samples has enormous implications on a) the reliability of classification and hence for instance the decision to discharge low risk patients after “rule-out”, and b) the cost effectiveness of the case management. Longer ED stay and observation and the collection of additional blood samples requires more staff time for nurses and physicians, and higher laboratory costs. In addition, prolonged ED stay is associated with dissatisfaction of all stakeholders.

The following table depicts the numbers of inappropriate “rule-out” classification (n=55) because a second blood draw was not obtained although required (exact time of chest pain onset unknown or below 3 hours). A misdiagnosis could potentially lead to a missed diagnosis of MI and unwarranted early discharge. In addition, the 2020 ESC Guidelines (contrasting to 2015 ESC Guidelines) mandate a third blood draw at 3 hours after the first blood draw for a definite diagnosis of patients initially classified into the “observational zone”. The table shows that a third blood draw was not collected in 77.6% of all patients in the “observational zone”. On the other hand, a large number of unnecessary blood draws was obtained despite a diagnostic first blood draw, both after initial “rule-out” (988 additional measurements) and after “rule-in” (442 additional measurements). Moreover, one or more additional blood samples were collected in 176 patients (9.9%) after a diagnostic set of two blood draws.

TABLE
Missing and excessive Troponin measurements

| | Missing, n (%) | Excessive^{§}, n (%) |
|---|---|---|
| All 0-hour protocol | 55/2306 (2.3) | 1430/2306 (62.0) |
| N = 2306 (46.7) | | |
| Diagnostic rule-out | n.a. | 988 (55.7) |
| n = 1774 (76.9) | n.a. | after rule-out |
| Diagnostic rule-in | | 442 (83.1) |
| n = 532 (23.1) | | after rule-in |
| Serial blood draws: | | |
| Diagnostic 2^{nd} blood draw | n.a. | 176/1773 (9.9) |
| Non-diagnostic 2^{nd} blood | 621/800 (77.6) | n.a. |
| draw** | | |

*A second blood draw beyond 0-h required due to missing info on CPO or CPO < 3 hours before first blood draw
**no third blood draw in cases classified into observational zone
^{§}n referring to patients, not measurements

### Example 5: Case Studies (Classification of Rule-Out, Observe or Rule-In)

### Patient 1

### Classification of a Patient

### Input:

Last symptom:
Oct. 18, 2019 18:59
Value of first hs-cTnT:
<5 ng/L
Timing of first hs-cTnT:
Oct. 18, 2019 19:32
Value of second hs-cTnT:
7 ng/L
Timing of second hs-cTnT:

### OUTPUT:

Rule-out (ESC 0/1 h)

Case interpretation: A patient with suspected ACS presents early after chest pain onset to hospital and receives a first blood draw 33 minutes after chest pain onset. The first cardiac troponin is below the LoD. However, the short period between CPO and first blood draw necessitates the collection of a second blood draw for rule-out of MI. Contrary, rule-in based on a single very high cardiac troponin result would not be affected by a short period from chest pain onset.

### Patient 2

### Classification of a Patient

### INPUT:

Last symptom:
Oct. 18, 2019 18:59
Value of first hs-cTnT:
7 ng/L
Timing of first hs-cTnT:
Oct. 18, 2019 23:32
Value of second hs-cTnT:
ng/L
Timing of second hs-cTnT:
Oct. 18, 2019 20:22

### OUTPUT:

Observation zone (ESC 0/1 h)

Case interpretation: The difference between first and second cardiac troponin is not <3 ng/L but exactly 3 ng/L. As such, the case is labelled as “observational zone” because neither criteria for rule-out nor for rule-in apply. 2020 ESC guidelines require a third blood draw at 3 hours to classify patients into “rule-out” or “rule-in”. The tool indicates the need for a third sample and proposes the correct (at 3 hours) or best possible time point (any time after 3 hours).

### Patient 3

### Classification of a Patient

### INPUT:

Last symptom:
Oct. 18, 2019 11:19
Value of first hs-cTnT:
<5 ng/L
Timing of first hs-cTnT:
Oct. 18, 2019 19:32
Value of second hs-cTnT:
Not available
Timing of second hs-cTnT:
Not available

### OUTPUT:

Rule-out (ESC 0 h)

Case interpretation: In the presence of a cardiac troponin value below the LoD and a period of more than 3 hours after chest pain onset, a single blood draw allows for a classification into “rule-out”. An additional blood draw is not required per 2020 ESC Guidelines.

### Patient 4

### Classification of a Patient

### INPUT:

Last symptom:
Oct. 18, 2019 18:59
Value of first hs-cTnT:
5 ng/L
Timing of first hs-cTnT:
Oct. 18, 2019 19:32
Value of second hs-cTnT:
Not available
Timing of second hs-cTnT:
Not available

### OUTPUT:

Protocol mandates second hs-cTnT

### Patient 5

Classification of a patient
Last symptom:
Oct. 18, 2019 18:59
Value of first hs-cTnT:
15 ng/L
Timing of first hs-cTnT:
Oct. 18, 2019 19:32
Value of second hs-cTnT:
19 ng/L
Timing of second hs-cTnT:
Oct. 18, 2019 22:32

### OUTPUT:

Rule-in (ESC 0/3 h)

Case interpretation: The first blood draw yields a cardiac troponinof 15 ng/L which is greater or equal to 12 ng/L as well as above the 99& percentile upper limit of normal for this assay (14 ng/L). As the time delay between the first and the second blood draw is 180 minutes, thereby exceeding the optimal time points (+tolerance time of 29 minutes) to apply the 0/1 hour (max 89 minutes) or the 0/2 hour protocol (max. 149 minutes), the 0/3 hour protocol has to be used. In the presence of a first cardiac troponin above the 99^{th} percentile upper limit of normal, a rise and or fall of 20 percent or more qualifies for rule-in.

FDA version

Case 6 is otherwise identical with case 5 but using FDA criteria instead of non-US criteria

### Patient 6

### Classification of a Patient

### INPUT:

Last symptom:
Oct. 18, 2019 18:59
Value of first hs-cTnT:
15 ng/L
Timing of first hs-cTnT:
Oct. 18, 2019 19:32
Value of second hs-cTnT:
19 ng/L
Timing of second hs-cTnT:
Oct. 18, 2019 22:32

### OUTPUT:

Single upper limit of normal: For both sexes: Rule-out (FDA 0/3 h)
Sex-specific cutoff: For male: Rule-out (FDA 0/3 h)
   For female: Rule-in (FDA 0/3 h)

Case interpretation: The case has a first cardiac troponin that is below the single sex-independent 99^{th} percentile upper reference limit that has been proposed for the US (19 ng/L). Due to a time interval of 3 hours between the first and the second blood draw, the 0/3 hour algorithm applies. As the diagnosis of MI requires a difference of more than 50% of the upper limit of normal, i.e. >9 ng/L, the case has to be triaged as “rule-out”, both in men and women. If sex-specific cutoffs are applied, the classification into “rule-out” does not change for the male. However, if the case is a female, the first cardiac troponin exceeds the upper limit of the sex-specific upper limit of normal. Therefore, a diagnosis of MI requires a rise and or fall of 20 percent or more. The second blood draw shows a rise by 4 ng/L and thus greater than 20%. In a female, the classification is “rule-in”.

### Case 7:

### Classification of a Patient

### INPUT:

Last symptom:
Oct. 18, 2019 08:30
Value of first hs-cTnT:
ng/L
Timing of first hs-cTnT:
Oct. 18, 2019 11:50
Value of second hs-cTnT:
Not available
Timing of second hs-cTnT:
Not available

### OUTPUT:

Rule-out (FDA 0 h)
Protocol mandates second hs-cTnT per non-US version

Case interpretation: In the presence of a first cardiac troponin below the US reporting limit, i.e. 6 ng/L and an interval to the first blood draw of more than 3 hours after chest pain onset, the case is triaged as “rule-out” using a single blood draw. Using the non-US application instead, would require a second blood draw because the first troponin is equal or above the limit of direction, i.e. 5 ng/L).

### Example 7: Comparison “Real World” Situation Versus “Ideal World”

While the ESC 0/1 hour algorithm had been introduced as early as 2015 and its use was encouraged, the proposed time intervals between blood collection could not be followed in real life conditions, particularly in crowding situations.

FIG. 5A shows The “ideal world” with management of patients with suspected ACS under conditions that are typical for controlled clinical trials. The figure shows findings from an observational trial on 2,146 patients where the second blood draw was timed exactly 60 minutes after the first blood draw using a stopwatch.

In the real life setting exact timing of blood draws is not feasible. In our study population on 4934 patients a heterogenous right-skewed distribution is observed in FIG. 5A.

As such it becomes apparent that adjustment for truly elapsed time between blood samples is important in clinical routine and outside of controlled trials to ensure a correct classification because hs-cTn concentrations increase in proportion with elapsed time after an MI and cutoffs and concentration changes have been established individually for all early protocols and for hs-cTnT and for each validated hs-cTnI assay.

### Example 7: Testing a Population

The yield of ML on the classification of the entire population into rule-out, observe zone, or rule-in is provided in a Table (showing anonymized patient ID, hs-cTnT baseline result and follow-up result where applicable, classification and annotation on the explicit rule that applied) is shown in the following Table D.

TABLE B
Classification of test population

| | Baseline | Follow up | |
|---|---|---|---|
| | hs-cTnT | hs-cTnT | |
| ID | [ng/L] | [ng/L] | Rule |
| 1 | 22 | 25 | Rule-out (ESC 0/3 h) |
| 2 | 13 | 8 | Observation zone (0/2 h, Reichlin 2015) |
| 3 | 4 | 5 | Rule-out (ESC 0/3 h) |
| 4 | 19 | 102 | Rule-in (ESC 0/3 h) |
| 5 | 42 | 35 | Rule-out (ESC 0/3 h) |
| 6 | 16 | 112 | Rule-in (0/2 h, Reichlin 2015) |
| 7 | 8 | 7 | Rule-out (0/2 h, Reichlin 2015) |
| 8 | 19 | 15 | Rule-in (ESC 0/3 h) |
| 9 | 409 | 486 | Rule-in (ESC 0 h) |
| 10 | 5 | 5 | Rule-out (ESC 0/1 h) |
| 11 | 6 | 6 | Rule-out (0/2 h, Reichlin 2015) |
| 12 | 6 | 6 | Rule-out (hsTnT and Copeptin 0 h, Moeckel 2014) |
| 13 | 15 | 13 | Rule-out (ESC 0/3 h) |
| 14 | 3 | | Rule-out (hsTnT and Copeptin 0 h, Moeckel 2014) |
| 15 | 10 | 11 | Rule-out (ESC 0/1 h) |
| 16 | 41 | 45 | Rule-out (ESC 0/3 h) |
| 17 | 29 | 28 | Observation zone (0/2 h, Reichlin 2015) |
| 18 | 6 | 8 | Rule-out (ESC 0 h) |
| 19 | 71 | 69 | Rule-in (ESC 0 h) |
| 20 | 8 | 7 | Rule-out (0/2 h, Reichlin 2015) |
| 21 | 7 | 8 | Rule-out (hsTnT and Copeptin 0 h, Mocckel 2014) |
| 22 | 13 | 8 | Observation zone (0/2 h, Reichlin 2015) |
| 23 | 25 | 24 | Rule-out (ESC 0/3 h) |
| 24 | 3 | | Rule-out (hsTnT and Copeptin 0 h, Moeckel 2014) |
| 25 | 5 | 5 | Rule-out (hsTnT and Copeptin 0 h, Mocckel 2014) |
| 26 | 1260 | 1092 | Rule-in (ESC 0 h) |
| 27 | 275 | 278 | Rule-in (ESC 0 h) |
| 28 | 4 | 1.5 | Rule-out (0/2 h, Reichlin 2015) |
| 29 | 11 | 11 | Rule-out (0/2 h, Reichlin 2015) |
| 30 | 49 | 314 | Rule-in (ESC 0/3 h) |
| 31 | 8 | 9 | Rule-out (hsTnT and Copcptin 0 h, Mocckel 2014) |
| 32 | 16 | 13 | Rule-out (ESC 0/3 h) |
| 33 | 18 | 20 | Observation zone (ESC 0/1 h) |
| 34 | 31 | 40 | Rule-in (ESC 0/3 h) |
| 35 | 32 | 31 | Rule-out (ESC 0/3 h) |
| 36 | 8 | 7 | Rule-out (ESC 0/1 h) |
| 37 | 17 | 17 | Rule-out (ESC 0/3 h) |
| 38 | 6 | 1.5 | Observation zone (ESC 0/1 h) |
| 39 | 11 | | Rule-out (hsTnT and Copeptin 0 h, Moeckel 2014) |
| 40 | 1.5 | | Rule-out (hsTnT and Copeptin 0 h, Moeckel 2014) |
| 41 | 39 | 30 | Rule-in (ESC 0/3 h) |
| 42 | 18 | 17 | Rule-out (ESC 0/3 h) |
| 43 | 19 | 16 | Observation zonc (ESC 0/1 h) |
| 44 | 21 | 21 | Observation zone (0/2 h, Reichlin 2015) |
| 45 | 366 | | Rule-in (ESC 0 h) |
| 46 | 27 | 80 | Rule-in (ESC 0/3 h) |
| 47 | 62 | 57 | Rule-in (ESC 0 h) |
| 48 | 3 | | Rule-out (ESC 0 h) |
| 49 | 78 | 878 | Rule-in (ESC 0 h) |
| 50 | 48 | 186 | Rule-in (ESC 0/3 h) |
| 51 | 12 | 11 | Rule-out (ESC 0/3 h) |
| 52 | 178 | | Rule-in (ESC 0 h) |
| 53 | 105 | | Rule-in (ESC 0 h) |
| 54 | 5 | 6 | Rule-out (0/2 h, Reichlin 2015) |
| 55 | 7 | | Rule-out (ESC 0 h) |
| 56 | 18 | 18 | Observation zone (ESC 0/1 h) |
| 57 | 50 | 56 | Rule-out (ESC 0/3 h) |
| 58 | 10 | 9 | Rule-out (0/2 h, Reichlin 2015) |
| 59 | 12 | 14 | Observation zonc (0/2 h, Reichlin 2015) |
| 60 | 24 | 37 | Rule-in (ESC 0/3 h) |
| 61 | 27 | 23 | Rule-out (ESC 0/3 h) |
| 62 | 11 | 8 | Rule-out (ESC 0/3 h) |
| 63 | 16 | 14 | Observation zone (ESC 0/1 h) |
| 64 | 8 | 7 | Rule-out (ESC 0 h) |
| 65 | 418 | 403 | Rule-in (ESC 0 h) |
| 66 | 22 | 24 | Rule-out (ESC 0/3 h) |
| 67 | 11 | 10 | Rule-out (hsTnT and Copeptin 0 h, Moeckel 2014) |
| 68 | 37 | 42 | Rule-out (ESC 0/3 h) |
| 69 | 365 | | Rule-in (ESC 0 h) |
| 70 | 4 | | Rule-out (ESC 0 h) |
| 71 | 10 | | Rule-out (ESC 0 h) |
| 72 | 10 | 8 | Rule-out (ESC 0 h) |
| 73 | 1.5 | | Rule-out (ESC 0 h) |
| 74 | 20 | 18 | Rule-out (ESC 0/3 h) |
| 75 | 21 | 36 | Rule-in (ESC 0/1 h) |
| 76 | 35 | 54 | Rule-in (ESC 0/3 h) |
| 77 | 19 | 12 | Rule-out (ESC 0/3 h) |
| 78 | 12 | | Rule-out (hsTnT and Copeptin 0 h, Moeckel 2014) |
| 79 | 10 | 10 | Rule-out (ESC 0/1 h) |
| 80 | 28 | 33 | Observation zone (0/2 h, Reichlin 2015) |
| 81 | 9 | 7 | Rule-out (ESC 0 h) |
| 82 | 6 | 5 | Rule-out (hsTnT and Copeptin 0 h, Moeckel 2014) |
| 83 | 14 | | Rule-out (hsTnT and Copeptin 0 h, Moeckel 2014) |
| 84 | 19 | 22 | Rule-out (ESC 0/3 h) |
| 85 | 11 | 11 | Rule-out (hsTnT and Copeptin 0 h, Moeckel 2014) |
| 86 | 367 | | Rule-in (ESC 0 h) |
| 87 | 14 | 13 | Rule-out (ESC 0/3 h) |
| 88 | 16 | 16 | Observation zone (0/2 h, Reichlin 2015) |
| 89 | 8 | 13 | Rule-in (ESC 0/1 h) |
| 90 | 12 | 8 | Observation zone (0/2 h, Reichlin 2015) |
| 91 | 9 | | Rule-out (hsTnT and Copeptin 0 h, Moeckel 2014) |
| 92 | 23 | 22 | Rule-out (ESC 0/3 h) |
| 93 | 8 | 7 | Rule-out (hsTnT and Copeptin 0 h, Moeckel 2014) |
| 94 | 16 | 16 | Rule-out (ESC 0/3 h) |
| 95 | 12 | 10 | Observation zone (ESC 0/1 h) |
| 96 | 7 | 10 | Rule-out (0/2 h, Reichlin 2015) |
| 97 | 5 | 8 | Rule-out (ESC 0 h) |
| 98 | 86 | 85 | Rule-in (ESC 0 h) |
| 99 | 8 | 9 | Rule-out (0/2 h, Reichlin 2015) |
| 100 | 9 | 8 | Rule-out (ESC 0 h) |
| 101 | 13 | 14 | Rule-out (ESC 0 h) |
| 102 | 7 | 6 | Rule-out (0/2 h, Reichlin 2015) |
| 103 | 6 | 8 | Rule-out (ESC 0/1 h) |
| 104 | 7 | 6 | Rule-out (0/2 h, Reichlin 2015) |
| 105 | 19 | 20 | Rule-out (ESC 0/3 h) |
| 106 | 9 | 8 | Rule-out (ESC 0 h) |
| 107 | 6 | 6 | Rule-out (ESC 0/3 h) |
| 108 | 1.5 | | Rule-out (ESC 0 h) |
| 109 | 8 | 8 | Rule-out (ESC 0 h) |
| 110 | 21 | 18 | Rule-out (ESC 0/3 h) |
| 111 | 5 | 5 | Rule-out (ESC 0 h) |
| 112 | 468 | | Rule-in (ESC 0 h) |
| 113 | 142 | 191 | Rule-in (ESC 0 h) |
| 114 | 4 | | Rule-out (hsTnT and Copeptin 0 h, Moeckel 2014) |
| 115 | 14 | | Observation zonc (ESC 0/1 h) |
| 116 | 332 | | Rule-in (ESC 0 h) |
| 117 | 1.5 | | Rule-out (ESC 0 h) |
| 118 | 10 | 13 | Rule-out (ESC 0/3 h) |
| 119 | 5 | 4 | Rule-out (ESC 0/1 h) |
| 120 | 5 | 7 | Rule-out (hsTnT and Copeptin 0 h, Moeckel 2014) |
| 121 | 24 | 28 | Rule-out (ESC 0/3 h) |
| 122 | 14 | 12 | Observation zone (ESC 0/1 h) |
| 123 | 10 | 4 | Observation zone (0/2 h, Reichlin 2015) |
| 124 | 11 | 12 | Rule-out (ESC 0 h) |
| 125 | 73 | 73 | Rule-in (ESC 0 h) |
| 126 | 21 | 52 | Rule-in (ESC 0/3 h) |
| 127 | 8 | 11 | Rule-out (ESC 0/3 h) |
| 128 | 9 | 11 | Rule-out (ESC 0/3 h) |
| 129 | 5 | | Rule-out (ESC 0 h) |
| 130 | 30 | 57 | Rule-in (ESC 0/3 h) |
| 131 | 189 | | Rule-in (ESC 0 h) |
| 132 | 4 | | Rule-out (ESC 0 h) |
| 133 | 24 | 23 | Observation zone (ESC 0/1 h) |
| 134 | 10 | | Rule-out (ESC 0 h) |
| 135 | 14 | | Rule-out (ESC 0 h) |
| 136 | 148 | | Rule-in (ESC 0 h) |
| 137 | 9 | 10 | Rule-out (ESC 0/1 h) |
| 138 | 5 | 5 | Rule-out (ESC 0/1 h) |
| 139 | 151 | 128 | Rule-in (ESC 0 h) |
| 140 | 11 | 8 | Rule-out (ESC 0/3 h) |
| 141 | 20 | 18 | Observation zone (0/2 h, Reichlin 2015) |
| 142 | 5 | 3 | Rule-out (hsTnT and Copeptin 0 h, Moeckel 2014) |
| 143 | 16 | 16 | Rule-out (ESC 0/3 h) |
| 144 | 36 | 34 | Rule-out (ESC 0/3 h) |
| 145 | 6 | 5 | Rule-out (ESC 0/1 h) |
| 146 | 7 | 5 | Rule-out (0/2 h, Reichlin 2015) |
| 147 | 4 | | Rule-out (ESC 0 h) |
| 148 | 5 | | Rule-out (ESC 0 h) |
| 149 | 26 | 27 | Observation zone (ESC 0/1 h) |
| 150 | 25 | 23 | Observation zone (ESC 0/1 h) |
| 151 | 16 | 17 | Observation zone (0/2 h, Reichlin 2015) |
| 152 | 15 | 15 | Observation zone (ESC 0/1 h) |
| 153 | 20 | 13 | Observation zone (0/2 h, Reichlin 2015) |
| 154 | 4 | | Rule-out (ESC 0 h) |
| 155 | 4 | | Rule-out (ESC 0 h) |
| 156 | 11 | 9 | Rule-out (ESC 0 h) |
| 157 | 12 | 10 | Observation zone (ESC 0/1 h) |
| 158 | 23 | 40 | Rule-in (ESC 0/3 h) |
| 159 | 21 | 19 | Rule-out (ESC 0/3 h) |
| 160 | 13 | | Rule-out (ESC 0 h) |
| 161 | 8 | 6 | Rule-out (ESC 0/3 h) |
| 162 | 4 | 4 | Rule-out (ESC 0 h) |
| 163 | 9 | 10 | Rule-out (ESC 0/1 h) |
| 164 | 12 | 10 | Rule-out (ESC 0 h) |
| 165 | 106 | 134 | Rule-in (ESC 0 h) |
| 166 | 41 | 37 | Rule-out (ESC 0/3 h) |
| 167 | 17 | 15 | Rule-out (ESC 0/3 h) |
| 168 | 25 | 23 | Rule-out (ESC 0/3 h) |
| 169 | 32 | 26 | Rule-out (ESC 0/3 h) |
| 170 | 4 | | Rule-out (hsTnT and Copeptin 0 h, Moeckel 2014) |
| 171 | 8 | 10 | Rule-out (ESC 0/1 h) |
| 172 | 22 | 20 | Observation zone (ESC 0/1 h) |
| 173 | 23 | 24 | Observation zone (ESC 0/1 h) |
| 174 | 4 | | Rule-out (ESC 0 h) |
| 175 | 7 | 7 | Rule-out (0/2 h, Reichlin 2015) |
| 176 | 5 | 4 | Rule-out (hsTnT and Copeptin 0 h, Moeckel 2014) |
| 177 | 15 | 24 | Rule-in (ESC 0/3 h) |
| 178 | 20 | 18 | Rule-out (ESC 0/3 h) |
| 179 | 5 | | Rule-out (hsTnT and Copeptin 0 h, Moeckel 2014) |
| 180 | 27 | 23 | Rule-out (ESC 0/3 h) |
| 181 | 6 | | Rule-out (ESC 0 h) |
| 182 | 1.5 | | Rule-out (ESC 0 h) |
| 183 | 111 | 106 | Rule-in (ESC 0 h) |
| 184 | 8 | | Rule-out (hsTnT and Copeptin 0 h, Moeckel 2014) |
| 185 | 11 | 10 | Rule-out (hsTnT and Copeptin 0 h, Moeckel 2014) |
| 186 | 6 | 6 | Rule-out (hsTnT and Copeptin 0 h, Moeckel 2014) |
| 187 | 14 | 14 | Rule-out (ESC 0 h) |
| 188 | 9 | 8 | Rule-out (ESC 0/1 h) |
| 189 | 26 | 30 | Rule-out (ESC 0/3 h) |
| 190 | 6 | 7 | Rule-out (ESC 0 h) |
| 191 | 12 | | Rule-out (ESC 0 h) |
| 192 | 9 | 11 | Rule-out (hsTnT and Copeptin 0 h, Moeckel 2014) |
| 193 | 696 | | Rule-in (ESC 0 h) |
| 194 | 14 | 13 | Rule-out (hsTnT and Copeptin 0 h, Moeckel 2014) |
| 195 | 1.5 | | Rule-out (hsTnT and Copeptin 0 h, Moeckel 2014) |
| 196 | 6 | | Rule-out (hsTnT and Copeptin 0 h, Moeckel 2014) |
| 197 | 12 | 14 | Observation zone (ESC 0/1 h) |
| 198 | 6 | 8 | Rule-out (ESC 0 h) |
| 199 | 68 | 72 | Rule-in (ESC 0 h) |
| 200 | 183 | 172 | Rule-in (ESC 0 h) |
| 201 | 8 | 8 | Rule-out (hsTnT and Copeptin 0 h, Moeckel 2014) |
| 202 | 5 | 1.5 | Observation zone (ESC 0/1 h) |
| 203 | 251 | | Rule-in (ESC 0 h) |
| 204 | 5 | 4 | Rule-out (ESC 0/1 h) |
| 205 | 6 | 5 | Rule-out (ESC 0 h) |
| 206 | 6 | 5 | Rule-out (ESC 0 h) |
| 207 | 14 | 13 | Rule-out (ESC 0/3 h) |
| 208 | 5 | 5 | Rule-out (ESC 0/1 h) |
| 209 | 26 | 28 | Rule-out (ESC 0/3 h) |
| 210 | 719 | | Rule-in (ESC 0 h) |
| 211 | 16 | 13 | Rule-in (ESC 0/3 h) |
| 212 | 33 | 59 | Rule-in (ESC 0/3 h) |
| 213 | 16 | 17 | Rule-out (ESC 0/3 h) |
| 214 | 14 | 11 | Observation zone (0/2 h, Reichlin 2015) |
| 215 | 6 | 1.5 | Observation zone (ESC 0/1 h) |
| 216 | 6 | 4 | Rule-out (hsTnT and Copeptin 0 h, Moeckel 2014) |
| 217 | 158 | | Rule-in (ESC 0 h) |
| 218 | 6 | | Rule-out (ESC 0 h) |
| 219 | 7 | 7 | Rule-out (ESC 0/1 h) |
| 220 | 14 | 13 | Observation zone (ESC 0/1 h) |
| 221 | 1.5 | | Rule-out (hsTnT and Copcptin 0 h, Mocckel 2014) |
| 222 | 6 | 10 | Observation zone (ESC 0/1 h) |
| 223 | 15 | 17 | Rule-out (ESC 0/3 h) |
| 224 | 9 | 10 | Rule-out (ESC 0/1 h) |
| 225 | 28 | 29 | Observation zone (0/2 h, Reichlin 2015) |
| 226 | 1.5 | | Rule-out (ESC 0 h) |
| 227 | 54 | 75 | Rule-in (ESC 0 h) |
| 228 | 8 | | Rule-out (ESC 0 h) |
| 229 | 27 | 38 | Rule-in (ESC 0/3 h) |
| 230 | 4 | | Rule-out (hsTnT and Copeptin 0 h, Moeckel 2014) |
| 231 | 5 | 7 | Rule-out (hsTnT and Copeptin 0 h, Moeckel 2014) |
| 232 | 9 | 10 | Rule-out (hsTnT and Copeptin 0 h, Moeckel 2014) |
| 233 | 17 | 13 | Observation zone (ESC 0/1 h) |
| 234 | 5 | 4 | Rule-out (ESC 0/1 h) |
| 235 | 4 | | Rule-out (hsTnT and Copeptin 0 h, Moeckel 2014) |
| 236 | 1905 | | Rule-in (ESC 0 h) |
| 237 | 28 | 31 | Rule-out (ESC 0/3 h) |
| 238 | 38 | 38 | Rule-out (ESC 0/3 h) |
| 239 | 40 | 36 | Observation zone (0/2 h, Reichlin 2015) |
| 240 | 3 | | Rule-out (hsTnT and Copeptin 0 h, Moeckel 2014) |
| 241 | 9 | 10 | Rule-out (ESC 0/3 h) |
| 242 | 5 | 6 | Rule-out (ESC 0/1 h) |
| 243 | 13 | 12 | Observation zone (ESC 0/1 h) |
| 244 | 7 | | Rule-out (hsTnT and Copeptin 0 h, Moeckel 2014) |
| 245 | 23 | 22 | Observation zone (0/2 h, Reichlin 2015) |
| 246 | 9 | 10 | Rule-out (ESC 0/1 h) |
| 247 | 29 | 26 | Rule-out (ESC 0/3 h) |
| 248 | 5 | 4 | Rule-out (ESC 0 h) |
| 249 | 4 | 4 | Rule-out (ESC 0/3 h) |
| 250 | 52 | 24 | Rule-in (ESC 0 h) |
| 251 | 29 | 338 | Rule-in (ESC 0/3 h) |
| 252 | 14 | 17 | Observation zone (0/2 h, Reichlin 2015) |
| 253 | 8 | 5 | Rule-out (0/2 h, Reichlin 2015) |
| 254 | 19 | 36 | Rule-in (0/2 h, Reichlin 2015) |
| 255 | 11 | 3 | Observation zonc (0/2 h, Reichlin 2015) |
| 256 | 504 | | Rule-in (ESC 0 h) |
| 257 | 40 | 38 | Observation zone (ESC 0/1 h) |
| 258 | 19 | 23 | Rule-in (ESC 0/3 h) |
| 259 | 15 | 16 | Observation zone (ESC 0/1 h) |
| 260 | 11 | 10 | Rule-out (ESC 0/1 h) |
| 261 | 12 | 16 | Rule-out (ESC 0 h) |
| 262 | 49 | 41 | Rule-out (ESC 0/3 h) |
| 263 | 20 | 18 | Rule-out (ESC 0/3 h) |
| 264 | 31 | 30 | Observation zone (0/2 h, Reichlin 2015) |
| 265 | 23 | 14 | Rule-out (ESC 0/3 h) |
| 266 | 222 | 1045 | Rule-in (ESC 0 h) |
| 267 | 29 | 39 | Rule-in (0/2 h, Reichlin 2015) |
| 268 | 13 | 11 | Rule-out (0/2 h, Reichlin 2015) |
| 269 | 5 | 3 | Rule-out (ESC 0 h) |
| 270 | 5 | 4 | Rule-out (ESC 0 h) |
| 271 | 8 | | Rule-out (hsTnT and Copeptin 0 h, Moeckel 2014) |
| 272 | 5 | | Rule-out (hsTnT and Copeptin 0 h, Moeckel 2014) |
| 273 | 14 | 17 | Rule-out (hsTnT and Copeptin 0 h, Moeckel 2014) |
| 274 | 146 | 181 | Rule-in (ESC 0 h) |
| 275 | 10 | 7 | Rule-out (ESC 0/3 h) |
| 276 | 12 | 11 | Rule-out (0/2 h, Reichlin 2015) |
| 277 | 15 | 15 | Observation zonc (0/2 h, Reichlin 2015) |
| 278 | 8 | | Rule-out (hsTnT and Copeptin 0 h, Moeckel 2014) |
| 279 | 27 | | Rule-in (ESC 0 h) |
| 280 | 1.5 | | Rule-out (ESC 0 h) |
| 281 | 7 | 8 | Rule-out (0/2 h, Reichlin 2015) |
| 282 | 1.5 | | Rule-out (ESC 0 h) |
| 283 | 26 | 20 | Rule-in (ESC 0/3 h) |
| 284 | 6 | 6 | Rule-out (0/2 h, Reichlin 2015) |
| 285 | 23 | 25 | Observation zone (ESC 0/1 h) |
| 286 | 47 | 29 | Rule-in (ESC 0/3 h) |
| 287 | 36 | 38 | Observation zone (0/2 h, Reichlin 2015) |
| 288 | 29 | 31 | Rule-out (ESC 0/3 h) |
| 289 | 7 | 8 | Rule-out (ESC 0/3 h) |
| 290 | 14 | 16 | Observation zone (0/2 h, Reichlin 2015) |
| 291 | 5 | 6 | Rule-out (0/2 h, Reichlin 2015) |
| 292 | 25 | 18 | Rule-in (ESC 0/3 h) |
| 293 | 5 | 5 | Rule-out (ESC 0/1 h) |
| 294 | 9 | 8 | Rule-out (0/2 h, Reichlin 2015) |
| 295 | 4 | | Rule-out (ESC 0 h) |
| 296 | 16 | 152 | Rule-in (ESC 0/3 h) |
| 297 | 19 | 19 | Rule-out (ESC 0/3 h) |
| 298 | 108 | 115 | Rule-in (ESC 0 h) |
| 299 | 21 | 29 | Rule-in (ESC 0/3 h) |
| 300 | 43 | 60 | Rule-in (ESC 0/3 h) |
| 301 | 37 | 38 | Observation zone (0/2 h, Reichlin 2015) |
| 302 | 86 | | Rule-in (ESC 0 h) |
| 303 | 17 | 16 | Rule-out (ESC 0/3 h) |
| 304 | 8 | 11 | Rule-out (hsTnT and Copeptin 0 h, Moeckel 2014) |
| 305 | 5 | 5 | Rule-out (hsTnT and Copcptin 0 h, Mocckel 2014) |
| 306 | 8 | 9 | Rule-out (0/2 h, Reichlin 2015) |
| 307 | 48 | 62 | Rule-in (0/2 h, Reichlin 2015) |
| 308 | 8 | 9 | Rule-out (ESC 0/3 h) |
| 309 | 151 | 654 | Rule-in (ESC 0 h) |
| 310 | 37 | 39 | Observation zone (ESC 0/1 h) |
| 311 | 15 | 12 | Observation zonc (ESC 0/1 h) |
| 312 | 13 | 12 | Observation zone (ESC 0/1 h) |
| 313 | 16 | 17 | Rule-out (ESC 0/3 h) |
| 314 | 16 | 12 | Rule-out (ESC 0/3 h) |
| 315 | 136 | | Rule-in (ESC 0 h) |
| 316 | 11 | | Rule-out (ESC 0 h) |
| 317 | 27 | 27 | Rule-out (ESC 0/3 h) |
| 318 | 34 | 23 | Rule-in (ESC 0/3 h) |
| 319 | 7 | 1.5 | Observation zone (0/2 h, Reichlin 2015) |
| 320 | 11 | 11 | Rule-out (0/2 h, Reichlin 2015) |
| 321 | 18 | 14 | Rule-out (ESC 0/3 h) |
| 322 | 97 | 95 | Rule-in (ESC 0 h) |
| 323 | 8 | 8 | Rule-out (ESC 0/1 h) |
| 324 | 9 | 8 | Rule-out (0/2 h, Reichlin 2015) |
| 325 | 25 | 24 | Rule-out (ESC 0/3 h) |
| 326 | 141 | 123 | Rule-in (ESC 0 h) |
| 327 | 9 | | Rule-out (hsTnT and Copeptin 0 h, Moeckel 2014) |
| 328 | 22 | 21 | Rule-out (ESC 0/3 h) |
| 329 | 14 | 12 | Observation zone (ESC 0/1 h) |
| 330 | 7 | | Rule-out (ESC 0 h) |
| 331 | 5 | 6 | Rule-out (ESC 0/3 h) |
| 332 | 97 | 140 | Rule-in (ESC 0 h) |
| 333 | 13 | 13 | Rule-out (ESC 0/3 h) |
| 334 | 5 | 4 | Rule-out (ESC 0/3 h) |
| 335 | 4 | 4 | Rule-out (ESC 0 h) |
| 336 | 30 | 20 | Rule-in (ESC 0/3 h) |
| 337 | 36 | 36 | Rule-out (ESC 0/3 h) |
| 338 | 166 | | Rule-in (ESC 0 h) |
| 339 | 10 | 11 | Rule-out (0/2 h, Reichlin 2015) |
| 340 | 29 | 29 | Rule-out (ESC 0/3 h) |
| 341 | 54 | 54 | Rule-in (ESC 0 h) |
| 342 | 7 | 6 | Rule-out (ESC 0 h) |
| 343 | 1.5 | | Rule-out (ESC 0 h) |
| 344 | 21 | 27 | Rule-in (ESC 0/3 h) |
| 345 | 7 | 5 | Rule-out (hsTnT and Copeptin 0 h, Moeckel 2014) |
| 346 | 22 | 23 | Rule-out (ESC 0/3 h) |
| 347 | 32 | 36 | Observation zone (0/2 h, Reichlin 2015) |
| 348 | 4 | | Rule-out (hsTnT and Copeptin 0 h, Moeckel 2014) |
| 349 | 1.5 | | Rule-out (hsTnT and Copeptin 0 h, Moeckel 2014) |
| 350 | 16 | 14 | Observation zone (0/2 h, Reichlin 2015) |
| 351 | 7 | 8 | Rule-out (ESC 0/1 h) |
| 352 | 33 | 36 | Observation zone (ESC 0/1 h) |
| 353 | 42 | 42 | Observation zone (0/2 h, Reichlin 2015) |
| 354 | 83 | 327 | Rule-in (ESC 0 h) |
| 355 | 12 | 11 | Rule-out (0/2 h, Reichlin 2015) |
| 356 | 1.5 | | Rule-out (ESC 0 h) |
| 357 | 18 | 17 | Rule-out (ESC 0/3 h) |
| 358 | 10 | 10 | Rule-out (ESC 0 h) |
| 359 | 8 | 8 | Rule-out (ESC 0/1 h) |
| 360 | 5 | 5 | Rule-out (ESC 0/1 h) |
| 361 | 6 | 23 | Rule-in (ESC 0/1 h) |
| 362 | 5 | 5 | Rule-out (ESC 0 h) |
| 363 | 4 | | Rule-out (ESC 0 h) |
| 364 | 16 | 15 | Rule-out (ESC 0/3 h) |
| 365 | 21 | 22 | Rule-out (ESC 0/3 h) |
| 366 | 8 | | Rule-out (ESC 0 h) |
| 367 | 16 | 5 | Rule-out (ESC 0/3 h) |
| 368 | 11 | 10 | Rule-out (ESC 0 h) |
| 369 | 14 | 11 | Rule-out (ESC 0/3 h) |
| 370 | 15 | 14 | Observation zone (0/2 h, Reichlin 2015) |
| 371 | 6 | | Rule-out (ESC 0 h) |
| 372 | 7 | 8 | Rule-out (hsTnT and Copeptin 0 h, Moeckel 2014) |
| 373 | 5 | | Rule-out (hsTnT and Copeptin 0 h, Moeckel 2014) |
| 374 | 14 | 12 | Observation zone (0/2 h, Reichlin 2015) |
| 375 | 9 | 9 | Rule-out (hsTnT and Copeptin 0 h, Moeckel 2014) |
| 376 | 58 | | Rule-in (ESC 0 h) |
| 377 | 4 | | Rule-out (ESC 0 h) |
| 378 | 8 | 8 | Rule-out (ESC 0 h) |
| 379 | 22 | 51 | Rule-in (ESC 0/3 h) |
| 380 | 9 | 8 | Rule-out (ESC 0/1 h) |
| 381 | 8 | 5 | Observation zone (ESC 0/1 h) |
| 382 | 6 | 5 | Rule-out (hsTnT and Copeptin 0 h, Moeckel 2014) |
| 383 | 47 | 35 | Rule-in (ESC 0/3 h) |
| 384 | 1.5 | | Rule-out (hsTnT and Copeptin 0 h, Moeckel 2014) |
| 385 | 4 | | Rule-out (ESC 0 h) |
| 386 | 459 | | Rule-in (ESC 0 h) |
| 387 | 38 | 35 | Observation zone (0/2 h, Reichlin 2015) |
| 388 | 13 | 11 | Rule-out (ESC 0 h) |
| 389 | 1.5 | | Rule-out (hsTnT and Copcptin 0 h, Mocckel 2014) |
| 390 | 5 | 7 | Rule-out (ESC 0 h) |
| 391 | 25 | 19 | Rule-in (ESC 0/3 h) |
| 392 | 69 | | Rule-in (ESC 0 h) |
| 393 | 4 | | Rule-out (ESC 0 h) |
| 394 | 16 | 16 | Rule-out (ESC 0/3 h) |
| 395 | 348 | 590 | Rule-in (ESC 0 h) |
| 396 | 8 | 10 | Rule-out (0/2 h, Reichlin 2015) |
| 397 | 20 | 19 | Observation zone (0/2 h, Reichlin 2015) |
| 398 | 8 | 8 | Rule-out (ESC 0 h) |
| 399 | 1.5 | | Rule-out (hsTnT and Copeptin 0 h, Moeckel 2014) |
| 400 | 106 | 148 | Rule-in (ESC 0 h) |
| 401 | 5 | 7 | Rule-out (ESC 0 h) |
| 402 | 1.5 | | Rule-out (ESC 0 h) |
| 403 | 29 | 23 | Rule-out (ESC 0/3 h) |
| 404 | 24 | 27 | Rule-out (ESC 0/3 h) |
| 405 | 5 | 6 | Rule-out (ESC 0/1 h) |
| 406 | 83 | 170 | Rule-in (ESC 0 h) |
| 407 | 4 | | Rule-out (hsTnT and Copcptin 0 h, Mocckel 2014) |
| 408 | 27 | 26 | Rule-out (ESC 0/3 h) |
| 409 | 101 | 141 | Rule-in (ESC 0 h) |
| 410 | 19 | 19 | Observation zone (0/2 h, Reichlin 2015) |
| 411 | 25 | 50 | Rule-in (ESC 0/3 h) |
| 412 | 1.5 | | Rule-out (ESC 0 h) |
| 413 | 13 | 85 | Rule-in (ESC 0/3 h) |
| 414 | 5 | 5 | Rule-out (ESC 0 h) |
| 415 | 1.5 | | Rule-out (ESC 0 h) |
| 416 | 5 | 8 | Observation zone (ESC 0/1 h) |
| 417 | 10 | 10 | Rule-out (ESC 0/1 h) |
| 418 | 24 | 18 | Rule-in (ESC 0/3 h) |
| 419 | 21 | 22 | Rule-out (ESC 0/3 h) |
| 420 | 3 | | Rule-out (hsTnT and Copeptin 0 h, Moeckel 2014) |
| 421 | 1.5 | | Rule-out (hsTnT and Copeptin 0 h, Moeckel 2014) |
| 422 | 30 | 26 | Observation zone (ESC 0/1 h) |
| 423 | 7 | 7 | Rule-out (0/2 h, Reichlin 2015) |
| 424 | 8 | 6 | Rule-out (hsTnT and Copeptin 0 h, Moeckel 2014) |
| 425 | 30 | 26 | Observation zone (ESC 0/1 h) |
| 426 | 76 | | Rule-in (ESC 0 h) |
| 427 | 560 | 448 | Rule-in (ESC 0 h) |
| 428 | 1.5 | | Rule-out (ESC 0 h) |
| 429 | 1.5 | | Rule-out (ESC 0 h) |
| 430 | 54 | 49 | Rule-in (ESC 0 h) |
| 431 | 4 | | Rule-out (hsTnT and Copeptin 0 h, Moeckel 2014) |
| 432 | 48 | 50 | Rule-out (ESC 0/3 h) |
| 433 | 51 | 46 | Rule-out (ESC 0/3 h) |
| 434 | 5 | 5 | Rule-out (hsTnT and Copeptin 0 h, Moeckel 2014) |
| 435 | 47 | 40 | Observation zone (0/2 h, Reichlin 2015) |
| 436 | 14 | 16 | Rule-out (ESC 0/3 h) |
| 437 | 13 | 14 | Observation zone (0/2 h, Reichlin 2015) |
| 438 | 8 | 6 | Rule-out (0/2 h, Reichlin 2015) |
| 439 | 27 | 29 | Rule-out (ESC 0/3 h) |
| 440 | 4 | | Rule-out (ESC 0 h) |
| 441 | 11 | 16 | Observation zone (0/2 h, Reichlin 2015) |
| 442 | 33 | 34 | Observation zone (0/2 h, Reichlin 2015) |
| 443 | 7 | | Rule-out (hsTnT and Copeptin 0 h, Moeckel 2014) |
| 444 | 8 | 9 | Rule-out (hsTnT and Copeptin 0 h, Moeckel 2014) |
| 445 | 37 | 42 | Rule-out (ESC 0/3 h) |
| 446 | 8 | | Rule-out (ESC 0 h) |
| 447 | 6 | | Rule-out (hsTnT and Copeptin 0 h, Moeckel 2014) |
| 448 | 121 | | Rule-in (ESC 0 h) |
| 449 | 3 | | Rule-out (hsTnT and Copeptin 0 h, Moeckel 2014) |
| 450 | 3 | | Rule-out (ESC 0 h) |
| 451 | 61 | 51 | Rule-in (ESC 0 h) |
| 452 | 46 | 35 | Rule-in (ESC 0/3 h) |
| 453 | 56 | 56 | Rule-in (ESC 0 h) |
| 454 | 4 | | Rule-out (ESC 0 h) |
| 455 | 27 | 26 | Observation zone (0/2 h, Reichlin 2015) |
| 456 | 1.5 | | Rule-out (ESC 0 h) |
| 457 | 1.5 | | Rule-out (hsTnT and Copeptin 0 h, Moeckel 2014) |
| 458 | 1.5 | | Rule-out (hsTnT and Copeptin 0 h, Moeckel 2014) |
| 459 | 24 | 23 | Observation zone (0/2 h, Reichlin 2015) |
| 460 | 52 | 47 | Rule-in (ESC 0 h) |
| 461 | 10 | 15 | Rule-in (ESC 0/1 h) |
| 462 | 16 | 16 | Observation zone (ESC 0/1 h) |
| 463 | 6 | 4 | Rule-out (ESC 0 h) |
| 464 | 7 | 5 | Rule-out (ESC 0 h) |
| 465 | 5 | 6 | Rule-out (ESC 0/1 h) |
| 466 | 7 | 6 | Rule-out (ESC 0 h) |
| 467 | 1.5 | | Rule-out (ESC 0 h) |
| 468 | 73 | 60 | Rule-in (ESC 0 h) |
| 469 | 6 | 40 | Rule-in (0/2 h, Reichlin 2015) |
| 470 | 1.5 | | Rule-out (ESC 0 h) |
| 471 | 18 | 21 | Rule-out (ESC 0/3 h) |
| 472 | 5 | 4 | Rule-out (0/2 h, Reichlin 2015) |
| 473 | 4 | | Rule-out (ESC 0 h) |
| 474 | 22 | 26 | Rule-out (ESC 0/3 h) |
| 475 | 13 | 12 | Observation zone (ESC 0/1 h) |
| 476 | 6 | 8 | Rule-out (hsTnT and Copeptin 0 h, Moeckel 2014) |
| 477 | 11 | 15 | Rule-out (hsTnT and Copeptin 0 h, Moeckel 2014) |
| 478 | 37 | 35 | Observation zone (ESC 0/1 h) |
| 479 | 10 | 10 | Rule-out (hsTnT and Copcptin 0 h, Mocckel 2014) |
| 480 | 160 | 178 | Rule-in (ESC 0 h) |
| 481 | 916 | 1016 | Rule-in (ESC 0 h) |
| 482 | 439 | 380 | Rule-in (ESC 0 h) |
| 483 | 19 | 18 | Rule-out (ESC 0/3 h) |
| 484 | 153 | 171 | Rule-in (ESC 0 h) |
| 485 | 65 | 68 | Rule-in (ESC 0 h) |
| 486 | 20 | 20 | Rule-out (ESC 0/3 h) |
| 487 | 60 | 65 | Rule-in (ESC 0 h) |
| 488 | 1.5 | | Rule-out (ESC 0 h) |
| 489 | 662 | 1429 | Rule-in (ESC 0 h) |
| 490 | 4 | 1.5 | Rule-out (hsTnT and Copeptin 0 h, Moeckel 2014) |
| 491 | 79 | 65 | Rule-in (ESC 0 h) |
| 492 | 6 | 5 | Rule-out (hsTnT and Copeptin 0 h, Moeckel 2014) |
| 493 | 7 | 8 | Rule-out (hsTnT and Copeptin 0 h, Moeckel 2014) |
| 494 | 5 | 1.5 | Observation zone (ESC 0/1 h) |
| 495 | 21 | 21 | Rule-out (ESC 0/3 h) |
| 496 | 1.5 | | Rule-out (ESC 0 h) |
| 497 | 3 | | Rule-out (ESC 0 h) |
| 498 | 19 | 58 | Rule-in (ESC 0/3 h) |
| 499 | 13 | 13 | Rule-out (ESC 0/3 h) |
| 500 | 22 | 19 | Observation zone (0/2 h, Reichlin 2015) |
| 501 | 6 | 8 | Rule-out (ESC 0/3 h) |
| 502 | 39 | 30 | Rule-in (ESC 0/3 h) |
| 503 | 6 | 6 | Rule-out (ESC 0 h) |
| 504 | 130 | | Rule-in (ESC 0 h) |
| 505 | 5 | 6 | Rule-out (0/2 h, Reichlin 2015) |
| 506 | 11 | 9 | Rule-out (ESC 0/1 h) |
| 507 | 82 | 77 | Rule-in (ESC 0 h) |
| 508 | 5 | 6 | Rule-out (ESC 0/1 h) |
| 509 | 8 | 8 | Rule-out (0/2 h, Reichlin 2015) |
| 510 | 14 | 13 | Observation zone (ESC 0/1 h) |
| 511 | 170 | 470 | Rule-in (ESC 0 h) |
| 512 | 35 | 53 | Rule-in (ESC 0/3 h) |
| 513 | 28 | 28 | Rule-out (ESC 0/3 h) |
| 514 | 6 | 4 | Rule-out (ESC 0 h) |
| 515 | 40 | 37 | Rule-out (ESC 0/3 h) |
| 516 | 9 | 15 | Observation zone (0/2 h, Reichlin 2015) |
| 517 | 7 | 9 | Rule-out (ESC 0 h) |
| 518 | 13 | 13 | Rule-out (ESC 0 h) |
| 519 | 1.5 | | Rule-out (ESC 0 h) |
| 520 | 1.5 | | Rule-out (ESC 0 h) |
| 521 | 40 | 382 | Rule-in (ESC 0/3 h) |
| 522 | 13 | 46 | Rule-in (ESC 0/3 h) |
| 523 | 27 | 22 | Rule-out (ESC 0/3 h) |
| 524 | 57 | 48 | Rule-in (ESC 0 h) |
| 525 | 325 | 355 | Rule-in (ESC 0 h) |
| 526 | 1.5 | | Rule-out (ESC 0 h) |
| 527 | 1.5 | | Rule-out (hsTnT and Copeptin 0 h, Moeckel 2014) |
| 528 | 26 | 26 | Rule-out (ESC 0/3 h) |
| 529 | 1.5 | | Rule-out (hsTnT and Copeptin 0 h, Mocckel 2014) |
| 530 | 35 | 34 | Rule-out (ESC 0/3 h) |
| 531 | 27 | 24 | Rule-out (ESC 0/3 h) |
| 532 | 6 | 7 | Rule-out (ESC 0 h) |
| 533 | 9 | 7 | Rule-out (0/2 h, Reichlin 2015) |
| 534 | 12 | 19 | Rule-in (ESC 0/3 h) |
| 535 | 8 | 7 | Rule-out (hsTnT and Copcptin 0 h, Mocckel 2014) |
| 536 | 3 | 4 | Rule-out (0/2 h, Reichlin 2015) |
| 537 | 5 | 5 | Rule-out (hsTnT and Copeptin 0 h, Moeckel 2014) |
| 538 | 4 | 3 | Rule-out (hsTnT and Copeptin 0 h, Moeckel 2014) |
| 539 | 3 | 1.5 | Rule-out (hsTnT and Copeptin 0 h, Moeckel 2014) |
| 540 | 17 | 36 | Rule-in (ESC 0/3 h) |
| 541 | 73 | 1471 | Rule-in (ESC 0 h) |
| 542 | 92 | | Rule-in (ESC 0 h) |
| 543 | 7 | 6 | Rule-out (hsTnT and Copeptin 0 h, Moeckel 2014) |
| 544 | 58 | 55 | Rule-in (ESC 0 h) |
| 545 | 24 | 25 | Rule-out (ESC 0/3 h) |
| 546 | 21 | 18 | Rule-out (ESC 0/3 h) |
| 547 | 14 | 13 | Observation zonc (ESC 0/1 h) |
| 548 | 3379 | 3138 | Rule-in (ESC 0 h) |
| 549 | 34 | 42 | Rule-in (ESC 0/3 h) |
| 550 | 19 | 22 | Rule-out (ESC 0/3 h) |
| 551 | 4 | 8 | Rule-out (ESC 0/3 h) |
| 552 | 6 | 7 | Rule-out (ESC 0/1 h) |
| 553 | 42 | 53 | Rule-in (ESC 0/3 h) |
| 554 | 4 | 5 | Rule-out (0/2 h, Reichlin 2015) |
| 555 | 39 | 35 | Rule-out (ESC 0/3 h) |
| 556 | 11 | 8 | Observation zone (ESC 0/1 h) |
| 557 | 10 | 9 | Rule-out (ESC 0 h) |
| 558 | 271 | 194 | Rule-in (ESC 0 h) |
| 559 | 11 | 13 | Rule-out (ESC 0/3 h) |
| 560 | 85 | 87 | Rule-in (ESC 0 h) |
| 561 | 4 | 6 | Rule-out (ESC 0/1 h) |
| 562 | 47 | 33 | Rule-in (0/2 h, Reichlin 2015) |
| 563 | 7 | 7 | Rule-out (ESC 0/1 h) |
| 564 | 39 | 40 | Rule-out (ESC 0/3 h) |
| 565 | 5 | | Rule-out (ESC 0 h) |
| 566 | 1.5 | | Rule-out (ESC 0 h) |
| 567 | 4 | | Rule-out (ESC 0 h) |
| 568 | 19 | 22 | Observation zone (ESC 0/1 h) |
| 569 | 16 | 18 | Observation zone (0/2 h, Reichlin 2015) |
| 570 | 4 | 5 | Rule-out (ESC 0 h) |
| 571 | 10 | 12 | Rule-out (ESC 0/1 h) |
| 572 | 4 | 6 | Rule-out (0/2 h, Reichlin 2015) |
| 573 | 4 | 1.5 | Rule-out (hsTnT and Copeptin 0 h, Moeckel 2014) |
| 574 | 3 | | Rule-out (hsTnT and Copeptin 0 h, Moeckel 2014) |
| 575 | 19 | 14 | Rule-out (ESC 0/3 h) |
| 576 | 3 | 749 | Rule-in (0/2 h, Reichlin 2015) |
| 577 | 6 | 5 | Rule-out (ESC 0 h) |
| 578 | 1.5 | | Rule-out (ESC 0 h) |
| 579 | 609 | 559 | Rule-in (ESC 0 h) |
| 580 | 14 | 17 | Rule-out (ESC 0/3 h) |
| 581 | 10 | 9 | Rule-out (ESC 0 h) |
| 582 | 6 | 4 | Rule-out (ESC 0 h) |
| 583 | 8 | 7 | Rule-out (ESC 0 h) |
| 584 | 1.5 | | Rule-out (ESC 0 h) |
| 585 | 1.5 | | Rule-out (ESC 0 h) |
| 586 | 7 | 6 | Rule-out (ESC 0/3 h) |
| 587 | 149 | 144 | Rule-in (ESC 0 h) |
| 588 | 4 | 4 | Rule-out (ESC 0 h) |
| 589 | 1.5 | | Rule-out (ESC 0 h) |
| 590 | 46 | 45 | Observation zone (0/2 h, Reichlin 2015) |
| 591 | 5 | | Rule-out (ESC 0 h) |
| 592 | 4 | | Rule-out (ESC 0 h) |
| 593 | 24 | 32 | Rule-in (ESC 0/3 h) |
| 594 | 12 | 29 | Rule-in (ESC 0/3 h) |
| 595 | 7 | 8 | Rule-out (0/2 h, Reichlin 2015) |
| 596 | 20 | 24 | Observation zone (ESC 0/1 h) |
| 597 | 9 | 10 | Rule-out (ESC 0/3 h) |
| 598 | 24 | 28 | Observation zone (0/2 h, Reichlin 2015) |
| 599 | 16 | 21 | Rule-in (ESC 0/1 h) |
| 600 | 51 | 50 | Rule-out (ESC 0/3 h) |
| 601 | 38 | 193 | Rule-in (ESC 0/3 h) |
| 602 | 4 | 1.5 | Rule-out (hsTnT and Copeptin 0 h, Moeckel 2014) |
| 603 | 9 | 9 | Rule-out (ESC 0 h) |
| 604 | 18 | 18 | Rule-out (ESC 0/3 h) |
| 605 | 1.5 | | Rule-out (ESC 0 h) |
| 606 | 28 | 22 | Rule-in (ESC 0/3 h) |
| 607 | 29 | 29 | Rule-out (ESC 0/3 h) |
| 608 | 59 | 55 | Rule-in (ESC 0 h) |
| 609 | 6 | 5 | Rule-out (hsTnT and Copeptin 0 h, Moeckel 2014) |
| 610 | 33 | 30 | Rule-out (ESC 0/3 h) |
| 611 | 1.5 | | Rule-out (ESC 0 h) |
| 612 | 5 | | Rule-out (ESC 0 h) |
| 613 | 1.5 | | Rule-out (ESC 0 h) |
| 614 | 13 | 13 | Rule-out (ESC 0/3 h) |
| 615 | 5 | 6 | Rule-out (0/2 h, Reichlin 2015) |
| 616 | 5 | 1.5 | Rule-out (0/2 h, Reichlin 2015) |
| 617 | 298 | 1296 | Rule-in (ESC 0 h) |
| 618 | 6 | 5 | Rule-out (ESC 0/1 h) |
| 619 | 39 | 41 | Rule-out (ESC 0/3 h) |
| 620 | 9 | 12 | Observation zone (ESC 0/1 h) |
| 621 | 15 | 16 | Rule-out (ESC 0/3 h) |
| 622 | 1.5 | | Rule-out (hsTnT and Copeptin 0 h, Moeckel 2014) |
| 623 | 5 | 9 | Observation zone (0/2 h, Reichlin 2015) |
| 624 | 30 | 36 | Rule-out (ESC 0/3 h) |
| 625 | 1.5 | | Rule-out (ESC 0 h) |
| 626 | 17 | 158 | Rule-in (ESC 0/3 h) |
| 627 | 1.5 | | Rule-out (ESC 0 h) |
| 628 | 23 | 22 | Rule-out (ESC 0/3 h) |
| 629 | 1.5 | | Rule-out (ESC 0 h) |
| 630 | 5 | 1.5 | Observation zone (ESC 0/1 h) |
| 631 | 1.5 | | Rule-out (ESC 0 h) |
| 632 | 98 | | Rule-in (ESC 0 h) |
| 633 | 6 | 4 | Rule-out (ESC 0 h) |
| 634 | 9 | 7 | Rule-out (ESC 0/1 h) |
| 635 | 16 | 18 | Rule-out (ESC 0/3 h) |
| 636 | 23 | 39 | Rule-in (ESC 0/3 h) |
| 637 | 7 | 17 | Rule-in (ESC 0/1 h) |
| 638 | 10 | | Rule-out (hsTnT and Copeptin 0 h, Moeckel 2014) |
| 639 | 457 | 418 | Rule-in (ESC 0 h) |
| 640 | 29 | 23 | Observation zone (0/2 h, Reichlin 2015) |
| 641 | 4 | 4 | Rule-out (0/2 h, Reichlin 2015) |
| 642 | 6 | 5 | Rule-out (ESC 0 h) |
| 643 | 10 | 17 | Rule-in (ESC 0/3 h) |
| 644 | 21 | 19 | Rule-out (ESC 0/3 h) |
| 645 | 4 | | Rule-out (ESC 0 h) |
| 646 | 10 | 7 | Rule-out (ESC 0 h) |
| 647 | 11 | 12 | Rule-out (ESC 0 h) |
| 648 | 4 | 5 | Rule-out (ESC 0 h) |
| 649 | 9 | 9 | Rule-out (ESC 0/1 h) |
| 650 | 1.5 | 1.5 | Rule-out (0/2 h, Reichlin 2015) |
| 651 | 28 | 70 | Rule-in (ESC 0/3 h) |
| 652 | 5 | 5 | Rule-out (hsTnT and Copeptin 0 h, Moeckel 2014) |
| 653 | 399 | 382 | Rule-in (ESC 0 h) |
| 654 | 4 | | Rule-out (ESC 0 h) |
| 655 | 8 | 8 | Rule-out (ESC 0/1 h) |
| 656 | 447 | | Rule-in (ESC 0 h) |
| 657 | 18 | 19 | Observation zone (0/2 h, Reichlin 2015) |
| 658 | 32 | 37 | Rule-in (ESC 0/1 h) |
| 659 | 270 | 268 | Rule-in (ESC 0 h) |
| 660 | 9 | 6 | Observation zone (ESC 0/1 h) |
| 661 | 39 | 44 | Observation zone (0/2 h, Reichlin 2015) |
| 662 | 13 | 14 | Rule-out (hsTnT and Copeptin 0 h, Moeckel 2014) |
| 663 | 8 | 5 | Observation zone (ESC 0/1 h) |
| 664 | 4 | | Rule-out (ESC 0 h) |
| 665 | 30 | 30 | Rule-out (ESC 0/3 h) |
| 666 | 10 | 13 | Rule-out (ESC 0 h) |
| 667 | 12 | 12 | Rule-out (ESC 0 h) |
| 668 | 10 | 12 | Rule-out (0/2 h, Reichlin 2015) |
| 669 | 4 | 4 | Rule-out (0/2 h, Reichlin 2015) |
| 670 | 21 | 20 | Rule-out (ESC 0/3 h) |
| 671 | 1.5 | | Rule-out (ESC 0 h) |
| 672 | 3 | 12 | Rule-in (ESC 0/1 h) |
| 673 | 4 | 5 | Rule-out (ESC 0 h) |
| 674 | 50 | 142 | Rule-in (ESC 0/3 h) |
| 675 | 5 | 6 | Rule-out (ESC 0/1 h) |
| 676 | 202 | | Rule-in (ESC 0 h) |
| 677 | 9 | 11 | Rule-out (ESC 0 h) |
| 678 | 4 | 1.5 | Rule-out (0/2 h, Reichlin 2015) |
| 679 | 8 | | Rule-out (ESC 0 h) |
| 680 | 3 | 7 | Observation zone (ESC 0/1 h) |
| 681 | 1.5 | | Rule-out (hsTnT and Copeptin 0 h, Moeckel 2014) |
| 682 | 27 | 30 | Observation zone (0/2 h, Reichlin 2015) |
| 683 | 13 | 7 | Observation zone (0/2 h, Reichlin 2015) |
| 684 | 34 | 30 | Observation zone (0/2 h, Reichlin 2015) |
| 685 | 23 | 35 | Rule-in (0/2 h, Reichlin 2015) |
| 686 | 14 | 10 | Observation zone (ESC 0/1 h) |
| 687 | 5 | | Rule-out (ESC 0 h) |
| 688 | 6 | 1.5 | Rule-out (ESC 0 h) |
| 689 | 22 | 25 | Rule-out (ESC 0/3 h) |
| 690 | 8 | 1.5 | Rule-out (ESC 0 h) |
| 691 | 3 | | Rule-out (ESC 0 h) |
| 692 | 46 | 37 | Rule-out (ESC 0/3 h) |
| 693 | 7 | 8 | Rule-out (ESC 0 h) |
| 694 | 4 | 4 | Rule-out (ESC 0 h) |
| 695 | 9 | 8 | Rule-out (ESC 0 h) |
| 696 | 17 | 17 | Observation zone (0/2 h, Reichlin 2015) |
| 697 | 50 | 50 | Observation zone (0/2 h, Reichlin 2015) |
| 698 | 3 | 1.5 | Rule-out (hsTnT and Copeptin 0 h, Moeckel 2014) |
| 699 | 4 | | Rule-out (ESC 0 h) |
| 700 | 1.5 | 1.5 | Rule-out (ESC 0/3 h) |
| 701 | 18 | 15 | Observation zone (0/2 h, Reichlin 2015) |
| 702 | 40 | 45 | Rule-out (ESC 0/3 h) |
| 703 | 9 | 6 | Observation zonc (ESC 0/1 h) |
| 704 | 194 | 194 | Rule-in (ESC 0 h) |
| 705 | 4 | 4 | Rule-out (hsTnT and Copeptin 0 h, Moeckel 2014) |
| 706 | 17 | 16 | Observation zone (0/2 h, Reichlin 2015) |
| 707 | 7 | 9 | Rule-out (ESC 0 h) |
| 708 | 5 | 6 | Rule-out (0/2 h, Reichlin 2015) |
| 709 | 100 | 245 | Rule-in (ESC 0 h) |
| 710 | 25 | 121 | Rule-in (ESC 0/3 h) |
| 711 | 4 | 5 | Rule-out (hsTnT and Copeptin 0 h, Moeckel 2014) |
| 712 | 24 | 19 | Rule-out (ESC 0/3 h) |
| 713 | 9 | 10 | Rule-out (hsTnT and Copeptin 0 h, Moeckel 2014) |
| 714 | 18 | 18 | Observation zone (0/2 h, Reichlin 2015) |
| 715 | 72 | 82 | Rule-in (ESC 0 h) |
| 716 | 5 | 9 | Observation zone (ESC 0/1 h) |
| 717 | 39 | 37 | Rule-out (ESC 0/3 h) |
| 718 | 39 | 37 | Rule-out (ESC 0/3 h) |
| 719 | 8 | 7 | Rule-out (hsTnT and Copeptin 0 h, Moeckel 2014) |
| 720 | 8 | 10 | Rule-out (0/2 h, Reichlin 2015) |
| 721 | 25 | 26 | Rule-out (ESC 0/3 h) |
| 722 | 25 | 20 | Rule-out (ESC 0/3 h) |
| 723 | 9 | 11 | Rule-out (ESC 0/1 h) |
| 724 | 15 | 12 | Observation zone (0/2 h, Reichlin 2015) |
| 725 | 10 | 12 | Rule-out (0/2 h, Reichlin 2015) |
| 726 | 24 | 106 | Rule-in (ESC 0/3 h) |
| 727 | 12 | 11 | Rule-out (0/2 h, Reichlin 2015) |
| 728 | 1.5 | | Rule-out (ESC 0 h) |
| 729 | 5 | | Rule-out (ESC 0 h) |
| 730 | 5 | 1.5 | Rule-out (0/2 h, Reichlin 2015) |
| 731 | 11 | 11 | Rule-out (0/2 h, Reichlin 2015) |
| 732 | 4 | 4 | Rule-out (ESC 0 h) |
| 733 | 11 | 10 | Rule-out (0/2 h, Reichlin 2015) |
| 734 | 9 | 9 | Rule-out (0/2 h, Reichlin 2015) |
| 735 | 54 | 40 | Rule-in (ESC 0 h) |
| 736 | 39 | 36 | Rule-out (ESC 0/3 h) |
| 737 | 9 | 6 | Rule-out (hsTnT and Copeptin 0 h, Moeckel 2014) |
| 738 | 1.5 | | Rule-out (ESC 0 h) |
| 739 | 9 | 10 | Rule-out (ESC 0/1 h) |
| 740 | 22 | 19 | Rule-out (ESC 0/3 h) |
| 741 | 21 | 42 | Rule-in (ESC 0/3 h) |
| 742 | 10 | 8 | Rule-out (ESC 0 h) |
| 743 | 9 | 7 | Rule-out (hsTnT and Copcptin 0 h, Mocckel 2014) |
| 744 | 28 | 41 | Rule-in (ESC 0/3 h) |
| 745 | 36 | 37 | Rule-out (ESC 0/3 h) |
| 746 | 7 | 6 | Rule-out (hsTnT and Copeptin 0 h, Moeckel 2014) |
| 747 | 5 | | Rule-out (ESC 0 h) |
| 748 | 7 | 8 | Rule-out (ESC 0/3 h) |
| 749 | 6 | 1.5 | Observation zone (0/2 h, Reichlin 2015) |
| 750 | 9 | | Rule-out (hsTnT and Copeptin 0 h, Moeckel 2014) |
| 751 | 22 | 22 | Rule-out (ESC 0/3 h) |
| 752 | 43 | 43 | Rule-out (ESC 0/3 h) |
| 753 | 1.5 | | Rule-out (ESC 0 h) |
| 754 | 72 | 68 | Rule-in (ESC 0 h) |
| 755 | 81 | 129 | Rule-in (ESC 0 h) |
| 756 | 38 | 43 | Rule-out (ESC 0/3 h) |
| 757 | 9 | 9 | Rule-out (ESC 0 h) |
| 758 | 5 | | Rule-out (ESC 0 h) |
| 759 | 22 | 42 | Rule-in (ESC 0/3 h) |
| 760 | 8 | 9 | Rule-out (ESC 0 h) |
| 761 | 37 | 42 | Rule-in (ESC 0/3 h) |
| 762 | 6 | 3 | Rule-out (ESC 0 h) |
| 763 | 6 | 3 | Observation zone (ESC 0/1 h) |
| 764 | 6 | 6 | Rule-out (ESC 0/3 h) |
| 765 | 4 | 5 | Rule-out (0/2 h, Reichlin 2015) |
| 766 | 206 | 371 | Rule-in (ESC 0 h) |
| 767 | 10 | 9 | Rule-out (0/2 h, Reichlin 2015) |
| 768 | 1.5 | | Rule-out (ESC 0 h) |
| 769 | 53 | 65 | Rule-in (ESC 0 h) |
| 770 | 6 | 7 | Rule-out (ESC 0/1 h) |
| 771 | 88 | 77 | Rule-in (ESC 0 h) |
| 772 | 43 | 114 | Rule-in (0/2 h, Reichlin 2015) |
| 773 | 6 | 1.5 | Observation zone (ESC 0/1 h) |
| 774 | 21 | 19 | Rule-out (ESC 0/3 h) |
| 775 | 29 | 27 | Rule-out (ESC 0/3 h) |
| 776 | 5 | 5 | Rule-out (hsTnT and Copeptin 0 h, Moeckel 2014) |
| 777 | 722 | 745 | Rule-in (ESC 0 h) |
| 778 | 8 | | Rule-out (ESC 0 h) |
| 779 | 188 | 178 | Rule-in (ESC 0 h) |
| 780 | 3 | | Rule-out (ESC 0 h) |
| 781 | 21 | 23 | Rule-out (ESC 0/3 h) |
| 782 | 9 | 13 | Rule-out (ESC 0 h) |
| 783 | 14 | 39 | Rule-in (0/2 h, Reichlin 2015) |
| 784 | 15 | 16 | Observation zone (0/2 h, Reichlin 2015) |
| 785 | 55 | 79 | Rule-in (ESC 0 h) |
| 786 | 8 | 11 | Observation zone (ESC 0/1 h) |
| 787 | 9 | 10 | Rule-out (0/2 h, Reichlin 2015) |
| 788 | 10 | 10 | Rule-out (ESC 0/1 h) |
| 789 | 5 | 3 | Rule-out (ESC 0/1 h) |
| 790 | 23 | 20 | Observation zone (0/2 h, Reichlin 2015) |
| 791 | 8 | 6 | Rule-out (ESC 0 h) |
| 792 | 289 | 245 | Rule-in (ESC 0 h) |
| 793 | 153 | 216 | Rule-in (ESC 0 h) |
| 794 | 20 | 23 | Rule-out (ESC 0/3 h) |
| 795 | 1.5 | | Rule-out (hsTnT and Copeptin 0 h, Moeckel 2014) |
| 796 | 7 | 6 | Rule-out (ESC 0 h) |
| 797 | 9 | 10 | Rule-out (0/2 h, Reichlin 2015) |
| 798 | 53 | 49 | Rule-in (ESC 0 h) |
| 799 | 52 | 333 | Rule-in (ESC 0 h) |
| 800 | 1.5 | | Rule-out (ESC 0 h) |
| 801 | 72 | 66 | Rule-in (ESC 0 h) |
| 802 | 6 | 8 | Rule-out (hsTnT and Copeptin 0 h, Moeckel 2014) |
| 803 | 12 | 14 | Observation zone (0/2 h, Reichlin 2015) |
| 804 | 7 | 4 | Rule-out (hsTnT and Copeptin 0 h, Moeckel 2014) |
| 805 | 6 | 6 | Rule-out (ESC 0/1 h) |
| 806 | 6 | 6 | Rule-out (hsTnT and Copeptin 0 h, Moeckel 2014) |
| 807 | 1.5 | | Rule-out (ESC 0 h) |
| 808 | 25 | 248 | Rule-in (ESC 0/3 h) |
| 809 | 8 | 9 | Rule-out (0/2 h, Reichlin 2015) |
| 810 | 9 | 8 | Rule-out (ESC 0 h) |
| 811 | 13 | 32 | Rule-in (ESC 0/3 h) |
| 812 | 8 | 5 | Observation zone (ESC 0/1 h) |
| 813 | 15 | 15 | Observation zone (ESC 0/1 h) |
| 814 | 36 | 24 | Rule-in (ESC 0/1 h) |
| 815 | 63 | 189 | Rule-in (ESC 0 h) |
| 816 | 1.5 | | Rule-out (ESC 0 h) |
| 817 | 117 | 116 | Rule-in (ESC 0 h) |
| 818 | 18 | 11 | Rule-out (ESC 0/3 h) |
| 819 | 3 | 1.5 | Rule-out (ESC 0/1 h) |
| 820 | 74 | 71 | Rule-in (ESC 0 h) |
| 821 | 13 | 1.5 | Rule-out (ESC 0/3 h) |
| 822 | 4 | | Rule-out (hsTnT and Copeptin 0 h, Moeckel 2014) |
| 823 | 63 | 62 | Rule-in (ESC 0 h) |
| 824 | 3 | | Rule-out (ESC 0 h) |
| 825 | 20 | 25 | Rule-in (ESC 0/3 h) |
| 826 | 6 | 39 | Rule-in (ESC 0/3 h) |
| 827 | 24 | 23 | Rule-out (ESC 0/3 h) |
| 828 | 5 | 9 | Observation zone (ESC 0/1 h) |
| 829 | 4 | | Rule-out (hsTnT and Copeptin 0 h, Moeckel 2014) |
| 830 | 11 | | Rule-out (ESC 0 h) |
| 831 | 18 | 16 | Observation zone (ESC 0/1 h) |
| 832 | 1.5 | | Rule-out (ESC 0 h) |
| 833 | 5 | 6 | Rule-out (ESC 0 h) |
| 834 | 5 | 1.5 | Observation zone (ESC 0/1 h) |
| 835 | 3 | | Rule-out (hsTnT and Copeptin 0 h, Moeckel 2014) |
| 836 | 377 | 478 | Rule-in (ESC 0 h) |
| 837 | 15 | 13 | Rule-out (ESC 0/3 h) |
| 838 | 8 | 6 | Rule-out (ESC 0 h) |
| 839 | 5 | 5 | Rule-out (hsTnT and Copeptin 0 h, Moeckel 2014) |
| 840 | 4 | | Rule-out (ESC 0 h) |
| 841 | 3 | | Rule-out (hsTnT and Copeptin 0 h, Moeckel 2014) |
| 842 | 4 | 1.5 | Rule-out (hsTnT and Copeptin 0 h, Moeckel 2014) |
| 843 | 3 | | Rule-out (hsTnT and Copcptin 0 h, Mocckel 2014) |
| 844 | 5 | 7 | Rule-out (hsTnT and Copeptin 0 h, Moeckel 2014) |
| 845 | 12 | 12 | Rule-out (ESC 0 h) |
| 846 | 17 | 20 | Rule-out (ESC 0/3 h) |
| 847 | 3 | | Rule-out (ESC 0 h) |
| 848 | 67 | 48 | Rule-in (ESC 0 h) |
| 849 | 12 | | Rule-out (ESC 0 h) |
| 850 | 1.5 | | Rule-out (hsTnT and Copeptin 0 h, Moeckel 2014) |
| 851 | 12 | 13 | Rule-out (ESC 0/3 h) |
| 852 | 1.5 | | Rule-out (ESC 0 h) |
| 853 | 3 | 1.5 | Rule-out (ESC 0/3 h) |
| 854 | 10 | 13 | Rule-out (ESC 0/3 h) |
| 855 | 10 | 10 | Rule-out (ESC 0/3 h) |
| 856 | 1.5 | | Rule-out (ESC 0 h) |
| 857 | 8 | | Rule-out (ESC 0 h) |
| 858 | 42 | 89 | Rule-in (0/2 h, Reichlin 2015) |
| 859 | 5 | 4 | Rule-out (ESC 0 h) |
| 860 | 11 | 11 | Rule-out (ESC 0/1 h) |
| 861 | 3 | 7 | Observation zone (ESC 0/1 h) |
| 862 | 4 | | Rule-out (ESC 0 h) |
| 863 | 15 | 16 | Rule-out (ESC 0/3 h) |
| 864 | 1.5 | | Rule-out (ESC 0 h) |
| 865 | 1.5 | | Rule-out (ESC 0 h) |
| 866 | 1.5 | | Rule-out (ESC 0 h) |
| 867 | 16 | 12 | Rule-out (ESC 0/3 h) |
| 868 | 9 | 8 | Rule-out (ESC 0/3 h) |
| 869 | 5 | | Rule-out (ESC 0 h) |
| 870 | 2524 | 2614 | Rule-in (ESC 0 h) |
| 871 | 13 | 11 | Observation zonc (ESC 0/1 h) |
| 872 | 11 | 10 | Rule-out (hsTnT and Copeptin 0 h, Moeckel 2014) |
| 873 | 5 | 1.5 | Rule-out (ESC 0 h) |
| 874 | 3 | | Rule-out (hsTnT and Copeptin 0 h, Moeckel 2014) |
| 875 | 16 | 16 | Observation zone (ESC 0/1 h) |
| 876 | 17 | 23 | Rule-in (ESC 0/1 h) |
| 877 | 4 | 1.5 | Rule-out (0/2 h, Reichlin 2015) |
| 878 | 1.5 | | Rule-out (ESC 0 h) |
| 879 | 6 | 3 | Rule-out (0/2 h, Reichlin 2015) |
| 880 | 11 | 9 | Rule-out (0/2 h, Reichlin 2015) |
| 881 | 6 | 4 | Rule-out (hsTnT and Copeptin 0 h, Moeckel 2014) |
| 882 | 8 | 7 | Rule-out (ESC 0 h) |
| 883 | 15 | 12 | Rule-out (ESC 0/3 h) |
| 884 | 9 | 1.5 | Observation zone (0/2 h, Reichlin 2015) |
| 885 | 19 | 117 | Rule-in (ESC 0/3 h) |
| 886 | 4 | | Rule-out (ESC 0 h) |
| 887 | 7 | 5 | Rule-out (ESC 0/3 h) |
| 888 | 40 | 231 | Rule-in (0/2 h, Reichlin 2015) |
| 889 | 69 | 63 | Rule-in (ESC 0 h) |
| 890 | 4 | 1.5 | Rule-out (ESC 0 h) |
| 891 | 37 | 80 | Rule-in (ESC 0/3 h) |
| 892 | 6 | 9 | Rule-out (ESC 0/3 h) |
| 893 | 8 | 6 | Rule-out (ESC 0 h) |
| 894 | 1.5 | | Rule-out (ESC 0 h) |
| 895 | 8 | 6 | Rule-out (hsTnT and Copeptin 0 h, Moeckel 2014) |
| 896 | 109 | 77 | Rule-in (ESC 0 h) |
| 897 | 25 | 26 | Observation zone (0/2 h, Reichlin 2015) |
| 898 | 11 | 9 | Rule-out (hsTnT and Copeptin 0 h, Moeckel 2014) |
| 899 | 143 | 282 | Rule-in (ESC 0 h) |
| 900 | 18 | 17 | Rule-out (ESC 0/3 h) |
| 901 | 16 | 19 | Rule-out (ESC 0/3 h) |
| 902 | 4 | | Rule-out (ESC 0 h) |
| 903 | 4 | | Rule-out (ESC 0 h) |
| 904 | 5 | 6 | Rule-out (ESC 0/1 h) |
| 905 | 4 | 1.5 | Rule-out (ESC 0/3 h) |
| 906 | 1.5 | 1.5 | Rule-out (0/2 h, Reichlin 2015) |
| 907 | 4 | | Rule-out (ESC 0 h) |
| 908 | 8 | 10 | Rule-out (ESC 0/3 h) |
| 909 | 29 | 84 | Rule-in (ESC 0/3 h) |
| 910 | 16 | 30 | Rule-in (0/2 h, Reichlin 2015) |
| 911 | 839 | | Rule-in (ESC 0 h) |
| 912 | 43 | 60 | Rule-in (ESC 0/3 h) |
| 913 | 296 | 341 | Rule-in (ESC 0 h) |
| 914 | 18 | 15 | Rule-out (ESC 0/3 h) |
| 915 | 154 | 482 | Rule-in (ESC 0 h) |
| 916 | 13 | 13 | Rule-out (ESC 0 h) |
| 917 | 9 | 5 | Observation zone (ESC 0/1 h) |
| 918 | 8 | 6 | Rule-out (ESC 0/1 h) |
| 919 | 10 | 9 | Rule-out (ESC 0/1 h) |
| 920 | 95 | 90 | Rule-in (ESC 0 h) |
| 921 | 31 | 27 | Rule-out (ESC 0/3 h) |
| 922 | 8 | 9 | Rule-out (hsTnT and Copeptin 0 h, Moeckel 2014) |
| 923 | 8 | 7 | Rule-out (ESC 0 h) |
| 924 | 4 | | Rule-out (hsTnT and Copeptin 0 h, Moeckel 2014) |
| 925 | 7 | 7 | Rule-out (hsTnT and Copeptin 0 h, Moeckel 2014) |
| 926 | 9 | 7 | Rule-out (hsTnT and Copeptin 0 h, Moeckel 2014) |
| 927 | 24 | 24 | Rule-out (ESC 0/3 h) |
| 928 | 67 | 72 | Rule-in (ESC 0 h) |
| 929 | 5 | 7 | Rule-out (ESC 0 h) |
| 930 | 1.5 | | Rule-out (ESC 0 h) |
| 931 | 158 | 156 | Rule-in (ESC 0 h) |
| 932 | 9 | 8 | Rule-out (ESC 0/3 h) |
| 933 | 3 | | Rule-out (hsTnT and Copeptin 0 h, Moeckel 2014) |
| 934 | 6 | 7 | Rule-out (hsTnT and Copeptin 0 h, Moeckel 2014) |
| 935 | 7 | 8 | Rule-out (hsTnT and Copeptin 0 h, Moeckel 2014) |
| 936 | 7 | 6 | Rule-out (hsTnT and Copeptin 0 h, Moeckel 2014) |
| 937 | 31 | 24 | Rule-in (ESC 0/3 h) |
| 938 | 40 | 45 | Rule-out (ESC 0/3 h) |
| 939 | 4 | 4 | Rule-out (ESC 0 h) |
| 940 | 28 | 19 | Observation zone (0/2 h, Reichlin 2015) |
| 941 | 4 | | Rule-out (ESC 0 h) |
| 942 | 21 | 24 | Rule-out (ESC 0/3 h) |
| 943 | 3 | | Rule-out (ESC 0 h) |
| 944 | 3 | | Rule-out (ESC 0 h) |
| 945 | 8 | 14 | Observation zone (0/2 h, Reichlin 2015) |
| 946 | 5 | 6 | Rule-out (0/2 h, Reichlin 2015) |
| 947 | 1.5 | | Rule-out (ESC 0 h) |
| 948 | 32 | 56 | Rule-in (ESC 0/3 h) |
| 949 | 8 | 8 | Rule-out (hsTnT and Copeptin 0 h, Mocckel 2014) |
| 950 | 30 | 28 | Rule-out (ESC 0/3 h) |
| 951 | 96 | 248 | Rule-in (ESC 0 h) |
| 952 | 1292 | | Rule-in (ESC 0 h) |
| 953 | 11 | 10 | Rule-out (ESC 0/1 h) |
| 954 | 7 | 8 | Rule-out (0/2 h, Reichlin 2015) |
| 955 | 5 | 1.5 | Rule-out (0/2 h, Reichlin 2015) |
| 956 | 4 | 3 | Rule-out (ESC 0 h) |
| 957 | 1.5 | | Rule-out (ESC 0 h) |
| 958 | 79 | 89 | Rule-in (ESC 0 h) |
| 959 | 4 | | Rule-out (ESC 0 h) |
| 960 | 34 | 28 | Rule-out (ESC 0/3 h) |
| 961 | 9 | 11 | Rule-out (ESC 0/1 h) |
| 962 | 5 | 4 | Rule-out (ESC 0 h) |
| 963 | 7 | 9 | Rule-out (0/2 h, Reichlin 2015) |
| 964 | 10 | 10 | Rule-out (0/2 h, Reichlin 2015) |
| 965 | 13 | 37 | Rule-in (ESC 0/1 h) |
| 966 | 8 | 10 | Rule-out (ESC 0/1 h) |
| 967 | 26 | 31 | Rule-out (ESC 0/3 h) |
| 968 | 17 | 25 | Rule-in (ESC 0/3 h) |
| 969 | 4 | 5 | Rule-out (ESC 0/1 h) |
| 970 | 1.5 | | Rule-out (ESC 0 h) |
| 971 | 63 | 71 | Rule-in (ESC 0 h) |
| 972 | 6 | 9 | Observation zone (ESC 0/1 h) |
| 973 | 1.5 | | Rule-out (ESC 0 h) |
| 974 | 1.5 | 4 | Rule-out (ESC 0/1 h) |
| 975 | 352 | | Rule-in (ESC 0 h) |
| 976 | 28 | 21 | Observation zone (0/2 h, Reichlin 2015) |
| 977 | 6 | 5 | Rule-out (ESC 0/1 h) |
| 978 | 5 | 5 | Rule-out (ESC 0/1 h) |
| 979 | 1.5 | | Rule-out (ESC 0 h) |
| 980 | 62 | | Rule-in (ESC 0 h) |
| 981 | 89 | 88 | Rule-in (ESC 0 h) |
| 982 | 109 | 258 | Rule-in (ESC 0 h) |
| 983 | 16 | 16 | Observation zonc (0/2 h, Reichlin 2015) |
| 984 | 1.5 | | Rule-out (ESC 0 h) |
| 985 | 7 | | Rule-out (hsTnT and Copeptin 0 h, Moeckel 2014) |
| 986 | 5 | | Rule-out (hsTnT and Copeptin 0 h, Moeckel 2014) |
| 987 | 11 | 11 | Rule-out (ESC 0 h) |
| 988 | 107 | | Rule-in (ESC 0 h) |
| 989 | 1.5 | | Rule-out (ESC 0 h) |
| 990 | 1.5 | | Rule-out (ESC 0 h) |
| 991 | 29 | 28 | Rule-out (ESC 0/3 h) |
| 992 | 8 | 9 | Rule-out (ESC 0/1 h) |
| 993 | 277 | 329 | Rule-in (ESC 0 h) |
| 994 | 8 | 6 | Rule-out (ESC 0 h) |
| 995 | 7 | 22 | Rule-in (ESC 0/1 h) |
| 996 | 7 | 7 | Rule-out (ESC 0/1 h) |
| 997 | 119 | 278 | Rule-in (ESC 0 h) |
| 998 | 1.5 | | Rule-out (ESC 0 h) |
| 999 | 5 | 4 | Rule-out (0/2 h, Reichlin 2015) |
| 1000 | 4 | 7 | Observation zone (ESC 0/1 h) |
| 1001 | 3 | 1.5 | Rule-out (0/2 h, Reichlin 2015) |
| 1002 | 40 | 38 | Observation zone (0/2 h, Reichlin 2015) |
| 1003 | 37 | 38 | Rule-out (ESC 0/3 h) |
| 1004 | 6 | 7 | Rule-out (ESC 0 h) |
| 1005 | 38 | 40 | Rule-out (ESC 0/3 h) |
| 1006 | 5 | 5 | Rule-out (ESC 0 h) |
| 1007 | 93 | | Rule-in (ESC 0 h) |
| 1008 | 44 | 44 | Rule-out (ESC 0/3 h) |
| 1009 | 11 | 10 | Rule-out (ESC 0 h) |
| 1010 | 6 | 6 | Rule-out (hsTnT and Copeptin 0 h, Moeckel 2014) |
| 1011 | 86 | 74 | Rule-in (ESC 0 h) |
| 1012 | 37 | 37 | Rule-out (ESC 0/3 h) |
| 1013 | 721 | | Rule-in (ESC 0 h) |
| 1014 | 129 | 122 | Rule-in (ESC 0 h) |
| 1015 | 8 | 9 | Rule-out (ESC 0/3 h) |
| 1016 | 61 | 63 | Rule-in (ESC 0 h) |
| 1017 | 4 | 4 | Rule-out (0/2 h, Reichlin 2015) |
| 1018 | 302 | 236 | Rule-in (ESC 0 h) |
| 1019 | 7 | 4 | Rule-out (ESC 0 h) |
| 1020 | 7 | 8 | Rule-out (ESC 0 h) |
| 1021 | 3 | 5 | Rule-out (ESC 0 h) |
| 1022 | 12 | 21 | Rule-in (ESC 0/3 h) |
| 1023 | 19 | 26 | Rule-in (ESC 0/3 h) |
| 1024 | 1.5 | | Rule-out (ESC 0 h) |
| 1025 | 5 | 5 | Rule-out (ESC 0 h) |
| 1026 | 17 | 32 | Rule-in (ESC 0/1 h) |
| 1027 | 1.5 | | Rule-out (ESC 0 h) |
| 1028 | 4 | | Rule-out (ESC 0 h) |
| 1029 | 34 | 33 | Rule-out (ESC 0/3 h) |
| 1030 | 7 | 12 | Observation zone (0/2 h, Reichlin 2015) |
| 1031 | 5 | 6 | Rule-out (ESC 0 h) |
| 1032 | 6 | 6 | Rule-out (ESC 0 h) |
| 1033 | 7 | 4 | Rule-out (0/2 h, Reichlin 2015) |
| 1034 | 16 | 20 | Rule-in (ESC 0/3 h) |
| 1035 | 4 | 5 | Rule-out (hsTnT and Copeptin 0 h, Moeckel 2014) |
| 1036 | 26 | 32 | Rule-in (ESC 0/3 h) |
| 1037 | 10 | 12 | Rule-out (hsTnT and Copeptin 0 h, Moeckel 2014) |
| 1038 | 32 | 34 | Observation zone (ESC 0/1 h) |
| 1039 | 74 | 60 | Rule-in (ESC 0 h) |
| 1040 | 51 | 49 | Rule-out (ESC 0/3 h) |
| 1041 | 74 | 67 | Rule-in (ESC 0 h) |
| 1042 | 33 | 37 | Observation zone (0/2 h, Reichlin 2015) |
| 1043 | 10 | 8 | Rule-out (ESC 0/1 h) |
| 1044 | 17 | 17 | Rule-out (ESC 0/3 h) |
| 1045 | 9 | 8 | Rule-out (0/2 h, Reichlin 2015) |
| 1046 | 4 | | Rule-out (ESC 0 h) |
| 1047 | 6 | 7 | Rule-out (ESC 0/3 h) |
| 1048 | 25 | 55 | Rule-in (0/2 h, Reichlin 2015) |
| 1049 | 1.5 | | Rule-out (ESC 0 h) |
| 1050 | 75 | 146 | Rule-in (ESC 0 h) |
| 1051 | 264 | 228 | Rule-in (ESC 0 h) |
| 1052 | 4 | | Rule-out (ESC 0 h) |
| 1053 | 15 | 14 | Rule-out (ESC 0/3 h) |
| 1054 | 10 | 9 | Rule-out (hsTnT and Copeptin 0 h, Moeckel 2014) |
| 1055 | 15 | 14 | Rule-out (ESC 0/3 h) |
| 1056 | 5 | 4 | Rule-out (ESC 0/1 h) |
| 1057 | 1.5 | | Rule-out (ESC 0 h) |
| 1058 | 9 | | Rule-out (ESC 0 h) |
| 1059 | 5 | 3 | Rule-out (ESC 0 h) |
| 1060 | 8 | 9 | Rule-out (ESC 0/1 h) |
| 1061 | 5 | 4 | Rule-out (0/2 h, Reichlin 2015) |
| 1062 | 319 | 474 | Rule-in (ESC 0 h) |
| 1063 | 3693 | 488 | Rule-in (ESC 0 h) |
| 1064 | 1.5 | | Rule-out (ESC 0 h) |
| 1065 | 21 | 35 | Rule-in (ESC 0/3 h) |
| 1066 | 13 | 15 | Rule-out (ESC 0/3 h) |
| 1067 | 1313 | 1479 | Rule-in (ESC 0 h) |
| 1068 | 4 | | Rule-out (ESC 0 h) |
| 1069 | 4 | 4 | Rule-out (ESC 0/1 h) |
| 1070 | 9 | 6 | Rule-out (0/2 h, Reichlin 2015) |
| 1071 | 846 | 829 | Rule-in (ESC 0 h) |
| 1072 | 132 | 183 | Rule-in (ESC 0 h) |
| 1073 | 109 | 106 | Rule-in (ESC 0 h) |
| 1074 | 86 | 71 | Rule-in (ESC 0 h) |
| 1075 | 7 | | Rule-out (ESC 0 h) |
| 1076 | 3 | | Rule-out (ESC 0 h) |
| 1077 | 3 | 3 | Rule-out (ESC 0/3 h) |
| 1078 | 94 | 90 | Rule-in (ESC 0 h) |
| 1079 | 64 | 79 | Rule-in (ESC 0 h) |
| 1080 | 1.5 | | Rule-out (ESC 0 h) |
| 1081 | 1.5 | | Rule-out (ESC 0 h) |
| 1082 | 7 | 9 | Rule-out (ESC 0 h) |
| 1083 | 5 | 4 | Rule-out (ESC 0/1 h) |
| 1084 | 6 | 5 | Rule-out (0/2 h, Reichlin 2015) |
| 1085 | 6 | 6 | Rule-out (ESC 0 h) |
| 1086 | 9 | 7 | Rule-out (ESC 0 h) |
| 1087 | 4 | | Rule-out (ESC 0 h) |
| 1088 | 36 | 41 | Rule-out (ESC 0/3 h) |
| 1089 | 9 | 8 | Rule-out (ESC 0 h) |
| 1090 | 7 | 7 | Rule-out (hsTnT and Copeptin 0 h, Moeckel 2014) |
| 1091 | 4 | | Rule-out (hsTnT and Copeptin 0 h, Moeckel 2014) |
| 1092 | 4 | | Rule-out (hsTnT and Copeptin 0 h, Moeckel 2014) |
| 1093 | 31 | 27 | Rule-out (ESC 0/3 h) |
| 1094 | 14 | 13 | Rule-out (hsTnT and Copeptin 0 h, Moeckel 2014) |
| 1095 | 32 | 33 | Rule-out (ESC 0/3 h) |
| 1096 | 8 | | Rule-out (hsTnT and Copeptin 0 h, Moeckel 2014) |
| 1097 | 50 | 53 | Rule-out (ESC 0/3 h) |
| 1098 | 98 | 118 | Rule-in (ESC 0 h) |
| 1099 | 8 | 8 | Rule-out (0/2 h, Reichlin 2015) |
| 1100 | 11 | 10 | Rule-out (ESC 0 h) |
| 1101 | 6 | 4 | Rule-out (ESC 0 h) |
| 1102 | 54 | 45 | Rule-in (ESC 0 h) |
| 1103 | 7 | 1.5 | Rule-out (ESC 0/3 h) |
| 1104 | 30 | 34 | Rule-out (ESC 0/3 h) |
| 1105 | 17 | 17 | Rule-out (ESC 0/3 h) |
| 1106 | 8 | 8 | Rule-out (hsTnT and Copeptin 0 h, Moeckel 2014) |
| 1107 | 15 | 20 | Rule-in (ESC 0/3 h) |
| 1108 | 98 | 147 | Rule-in (ESC 0 h) |
| 1109 | 4 | 5 | Rule-out (ESC 0/3 h) |
| 1110 | 6 | 9 | Rule-out (ESC 0 h) |
| 1111 | 7 | | Rule-out (hsTnT and Copeptin 0 h, Moeckel 2014) |
| 1112 | 30 | 30 | Observation zone (0/2 h, Reichlin 2015) |
| 1113 | 1.5 | 1.5 | Rule-out (ESC 0 h) |
| 1114 | 5 | 4 | Rule-out (hsTnT and Copeptin 0 h, Moeckel 2014) |
| 1115 | 7 | | Rule-out (hsTnT and Copeptin 0 h, Moeckel 2014) |
| 1116 | 1.5 | | Rule-out (hsTnT and Copeptin 0 h, Moeckel 2014) |
| 1117 | 5 | | Rule-out (ESC 0 h) |
| 1118 | 23 | 23 | Observation zone (0/2 h, Reichlin 2015) |
| 1119 | 1.5 | | Rule-out (ESC 0 h) |
| 1120 | 16 | 14 | Observation zone (ESC 0/1 h) |
| 1121 | 14 | 29 | Rule-in (ESC 0/1 h) |
| 1122 | 83 | | Rule-in (ESC 0 h) |
| 1123 | 18 | 19 | Rule-out (ESC 0/3 h) |
| 1124 | 4 | 4 | Rule-out (hsTnT and Copeptin 0 h, Moeckel 2014) |
| 1125 | 5 | | Rule-out (hsTnT and Copeptin 0 h, Moeckel 2014) |
| 1126 | 3 | 1.5 | Rule-out (hsTnT and Copeptin 0 h, Moeckel 2014) |
| 1127 | 103 | 93 | Rule-in (ESC 0 h) |
| 1128 | 44 | 47 | Rule-out (ESC 0/3 h) |
| 1129 | 1.5 | | Rule-out (ESC 0 h) |
| 1130 | 22 | 30 | Rule-in (ESC 0/3 h) |
| 1131 | 4 | | Rule-out (ESC 0 h) |
| 1132 | 1.5 | | Rule-out (ESC 0 h) |
| 1133 | 14 | 638 | Rule-in (ESC 0/3 h) |
| 1134 | 14 | 13 | Observation zone (0/2 h, Reichlin 2015) |
| 1135 | 5 | 4 | Rule-out (0/2 h, Reichlin 2015) |
| 1136 | 19 | 18 | Observation zone (ESC 0/1 h) |
| 1137 | 11 | 11 | Rule-out (ESC 0/3 h) |
| 1138 | 20 | 20 | Rule-out (ESC 0/3 h) |
| 1139 | 18 | 20 | Observation zone (0/2 h, Reichlin 2015) |
| 1140 | 12 | 10 | Rule-out (ESC 0 h) |
| 1141 | 12 | 12 | Rule-out (ESC 0 h) |
| 1142 | 10 | 14 | Rule-in (ESC 0/3 h) |
| 1143 | 11 | 10 | Rule-out (0/2 h, Reichlin 2015) |
| 1144 | 17 | 17 | Observation zone (0/2 h, Reichlin 2015) |
| 1145 | 6 | 5 | Rule-out (ESC 0 h) |
| 1146 | 5 | 4 | Rule-out (hsTnT and Copeptin 0 h, Moeckel 2014) |
| 1147 | 17 | 15 | Rule-out (ESC 0/3 h) |
| 1148 | 7 | 4 | Observation zone (ESC 0/1 h) |
| 1149 | 6 | 7 | Rule-out (ESC 0 h) |
| 1150 | 6 | 5 | Rule-out (0/2 h, Reichlin 2015) |
| 1151 | 29 | 27 | Observation zone (0/2 h, Reichlin 2015) |
| 1152 | 4 | | Rule-out (ESC 0 h) |
| 1153 | 6 | | Rule-out (ESC 0 h) |
| 1154 | 11 | 18 | Rule-out (ESC 0 h) |
| 1155 | 7 | 1.5 | Observation zone (0/2 h, Reichlin 2015) |
| 1156 | 8 | 6 | Rule-out (ESC 0/3 h) |
| 1157 | 1.5 | 3 | Rule-out (hsTnT and Copeptin 0 h, Moeckel 2014) |
| 1158 | 39 | 42 | Observation zone (0/2 h, Reichlin 2015) |
| 1159 | 31 | 25 | Rule-out (ESC 0/3 h) |
| 1160 | 27 | 57 | Rule-in (0/2 h, Reichlin 2015) |
| 1161 | 3 | 4 | Rule-out (ESC 0/3 h) |
| 1162 | 8 | 7 | Rule-out (ESC 0 h) |
| 1163 | 21 | 21 | Observation zonc (0/2 h, Reichlin 2015) |
| 1164 | 5 | 7 | Rule-out (hsTnT and Copeptin 0 h, Moeckel 2014) |
| 1165 | 4 | 5 | Rule-out (0/2 h, Reichlin 2015) |
| 1166 | 60 | 53 | Rule-in (ESC 0 h) |
| 1167 | 4 | | Rule-out (ESC 0 h) |
| 1168 | 36 | 43 | Rule-out (ESC 0/3 h) |
| 1169 | 1.5 | 3 | Rule-out (hsTnT and Copeptin 0 h, Moeckel 2014) |
| 1170 | 8 | 7 | Rule-out (ESC 0 h) |
| 1171 | 98 | 85 | Rule-in (ESC 0 h) |
| 1172 | 13 | 16 | Rule-out (ESC 0 h) |
| 1173 | 46 | 44 | Observation zonc (ESC 0/1 h) |
| 1174 | 1.5 | | Rule-out (ESC 0 h) |
| 1175 | 14 | 16 | Rule-out (ESC 0 h) |
| 1176 | 7 | 7 | Rule-out (hsTnT and Copeptin 0 h, Moeckel 2014) |
| 1177 | 10 | 13 | Observation zone (ESC 0/1 h) |
| 1178 | 17 | 16 | Observation zone (0/2 h, Reichlin 2015) |
| 1179 | 5 | 7 | Rule-out (ESC 0 h) |
| 1180 | 6 | 6 | Rule-out (ESC 0/1 h) |
| 1181 | 23 | 19 | Observation zone (0/2 h, Reichlin 2015) |
| 1182 | 6 | 6 | Rule-out (0/2 h, Reichlin 2015) |
| 1183 | 7 | 6 | Rule-out (0/2 h, Reichlin 2015) |
| 1184 | 20 | 19 | Rule-out (ESC 0/3 h) |
| 1185 | 3 | | Rule-out (ESC 0 h) |
| 1186 | 4 | | Rule-out (ESC 0 h) |
| 1187 | 56 | 52 | Rule-in (ESC 0 h) |
| 1188 | 7 | 6 | Rule-out (ESC 0/3 h) |
| 1189 | 26 | 24 | Rule-out (ESC 0/3 h) |
| 1190 | 33 | 69 | Rule-in (ESC 0/3 h) |
| 1191 | 25 | 48 | Rule-in (ESC 0/3 h) |
| 1192 | 10 | 11 | Rule-out (ESC 0 h) |
| 1193 | 28 | 30 | Observation zone (ESC 0/1 h) |
| 1194 | 25 | 60 | Rule-in (ESC 0/3 h) |
| 1195 | 11 | 8 | Rule-out (0/2 h, Reichlin 2015) |
| 1196 | 9 | 10 | Rule-out (0/2 h, Reichlin 2015) |
| 1197 | 7 | 6 | Rule-out (ESC 0 h) |
| 1198 | 6 | 7 | Rule-out (ESC 0/1 h) |
| 1199 | 1.5 | | Rule-out (ESC 0 h) |
| 1200 | 5 | 4 | Rule-out (ESC 0 h) |
| 1201 | 6 | 4 | Rule-out (ESC 0 h) |
| 1202 | 65 | 58 | Rule-in (ESC 0 h) |
| 1203 | 21 | 15 | Observation zone (0/2 h, Reichlin 2015) |
| 1204 | 9 | 10 | Rule-out (ESC 0 h) |
| 1205 | 12 | 10 | Rule-out (0/2 h, Reichlin 2015) |
| 1206 | 5 | | Rule-out (ESC 0 h) |
| 1207 | 47 | 46 | Observation zonc (ESC 0/1 h) |
| 1208 | 10 | 7 | Observation zone (ESC 0/1 h) |
| 1209 | 29 | 24 | Rule-out (ESC 0/3 h) |
| 1210 | 6 | 5 | Rule-out (0/2 h, Reichlin 2015) |
| 1211 | 11 | 8 | Rule-out (0/2 h, Reichlin 2015) |
| 1212 | 6 | 8 | Rule-out (ESC 0 h) |
| 1213 | 22 | 18 | Rule-out (ESC 0/3 h) |
| 1214 | 46 | 61 | Rule-in (0/2 h, Reichlin 2015) |
| 1215 | 7 | 6 | Rule-out (ESC 0/1 h) |
| 1216 | 29 | 29 | Observation zone (0/2 h, Reichlin 2015) |
| 1217 | 55 | 54 | Rule-in (ESC 0 h) |
| 1218 | 4 | 4 | Rule-out (ESC 0/3 h) |
| 1219 | 10 | 8 | Rule-out (ESC 0 h) |
| 1220 | 7 | 8 | Rule-out (hsTnT and Copeptin 0 h, Moeckel 2014) |
| 1221 | 43 | 136 | Rule-in (ESC 0/3 h) |
| 1222 | 29 | 30 | Observation zone (ESC 0/1 h) |
| 1223 | 10 | 8 | Rule-out (ESC 0 h) |
| 1224 | 13 | 176 | Rule-in (0/2 h, Reichlin 2015) |
| 1225 | 4 | | Rule-out (ESC 0 h) |
| 1226 | 4 | 6 | Rule-out (ESC 0 h) |
| 1227 | 9 | 9 | Rule-out (0/2 h, Reichlin 2015) |
| 1228 | 36 | 35 | Observation zone (0/2 h, Reichlin 2015) |
| 1229 | 16 | 15 | Rule-out (ESC 0/3 h) |
| 1230 | 10 | 8 | Rule-out (ESC 0 h) |
| 1231 | 4 | | Rule-out (ESC 0 h) |
| 1232 | 1.5 | | Rule-out (hsTnT and Copeptin 0 h, Moeckel 2014) |
| 1233 | 3 | | Rule-out (hsTnT and Copeptin 0 h, Moeckel 2014) |
| 1234 | 7 | | Rule-out (hsTnT and Copeptin 0 h, Moeckel 2014) |
| 1235 | 7 | 5 | Rule-out (ESC 0 h) |
| 1236 | 3 | 5 | Rule-out (ESC 0 h) |
| 1237 | 15 | 14 | Observation zone (0/2 h, Reichlin 2015) |
| 1238 | 4 | | Rule-out (ESC 0 h) |
| 1239 | 7 | 12 | Rule-out (ESC 0/3 h) |
| 1240 | 24 | 25 | Observation zone (0/2 h, Reichlin 2015) |
| 1241 | 5 | | Rule-out (ESC 0 h) |
| 1242 | 25 | 40 | Rule-in (ESC 0/3 h) |
| 1243 | 17 | 12 | Observation zone (0/2 h, Reichlin 2015) |
| 1244 | 129 | 291 | Rule-in (ESC 0 h) |
| 1245 | 9 | 9 | Rule-out (0/2 h, Reichlin 2015) |
| 1246 | 22 | 46 | Rule-in (0/2 h, Reichlin 2015) |
| 1247 | 88 | 76 | Rule-in (ESC 0 h) |
| 1248 | 11 | | Rule-out (ESC 0 h) |
| 1249 | 28 | 27 | Observation zone (ESC 0/1 h) |
| 1250 | 523 | 462 | Rule-in (ESC 0 h) |
| 1251 | 15 | 23 | Rule-in (ESC 0/3 h) |
| 1252 | 200 | | Rule-in (ESC 0 h) |
| 1253 | 4 | | Rule-out (ESC 0 h) |
| 1254 | 5 | | Rule-out (ESC 0 h) |
| 1255 | 5 | 9 | Observation zone (0/2 h, Reichlin 2015) |
| 1256 | 139 | 553 | Rule-in (ESC 0 h) |
| 1257 | 59 | 74 | Rule-in (ESC 0 h) |
| 1258 | 691 | 3790 | Rule-in (ESC 0 h) |
| 1259 | 68 | 75 | Rule-in (ESC 0 h) |
| 1260 | 4 | | Rule-out (ESC 0 h) |
| 1261 | 5 | 4 | Rule-out (hsTnT and Copeptin 0 h, Moeckel 2014) |
| 1262 | 57 | 66 | Rule-in (ESC 0 h) |
| 1263 | 8 | 7 | Rule-out (ESC 0 h) |
| 1264 | 9 | | Rule-out (ESC 0 h) |
| 1265 | 4 | 5 | Rule-out (0/2 h, Reichlin 2015) |
| 1266 | 10 | 11 | Rule-out (ESC 0 h) |
| 1267 | 6 | 5 | Rule-out (ESC 0/3 h) |
| 1268 | 360 | 426 | Rule-in (ESC 0 h) |
| 1269 | 8 | 8 | Rule-out (ESC 0/1 h) |
| 1270 | 19 | 18 | Observation zone (ESC 0/1 h) |
| 1271 | 25 | 33 | Rule-in (ESC 0/1 h) |
| 1272 | 7 | 8 | Rule-out (hsTnT and Copeptin 0 h, Moeckel 2014) |
| 1273 | 5 | 5 | Rule-out (ESC 0 h) |
| 1274 | 8 | 7 | Rule-out (ESC 0/1 h) |
| 1275 | 7 | 7 | Rule-out (hsTnT and Copeptin 0 h, Moeckel 2014) |
| 1276 | 306 | 278 | Rule-in (ESC 0 h) |
| 1277 | 8 | 7 | Rule-out (hsTnT and Copeptin 0 h, Moeckel 2014) |
| 1278 | 6 | 5 | Rule-out (ESC 0 h) |
| 1279 | 23 | 22 | Observation zonc (0/2 h, Reichlin 2015) |
| 1280 | 44 | 44 | Observation zone (ESC 0/1 h) |
| 1281 | 117 | 149 | Rule-in (ESC 0 h) |
| 1282 | 3 | 1.5 | Rule-out (ESC 0 h) |
| 1283 | 7 | 6 | Rule-out (ESC 0 h) |
| 1284 | 11 | 11 | Rule-out (ESC 0 h) |
| 1285 | 83 | 61 | Rule-in (ESC 0 h) |
| 1286 | 5 | 5 | Rule-out (ESC 0/1 h) |
| 1287 | 5 | 4 | Rule-out (ESC 0/1 h) |
| 1288 | 16 | 16 | Observation zone (0/2 h, Reichlin 2015) |
| 1289 | 25 | 23 | Observation zonc (0/2 h, Reichlin 2015) |
| 1290 | 22 | 45 | Rule-in (ESC 0/1 h) |
| 1291 | 28 | 27 | Observation zone (ESC 0/1 h) |
| 1292 | 4 | 4 | Rule-out (ESC 0 h) |
| 1293 | 18 | 16 | Observation zone (0/2 h, Reichlin 2015) |
| 1294 | 7 | 7 | Rule-out (ESC 0/1 h) |
| 1295 | 25 | 25 | Observation zonc (0/2 h, Reichlin 2015) |
| 1296 | 25 | 24 | Observation zone (0/2 h, Reichlin 2015) |
| 1297 | 11 | 11 | Rule-out (0/2 h, Reichlin 2015) |
| 1298 | 11 | 8 | Observation zone (ESC 0/1 h) |
| 1299 | 8 | 8 | Rule-out (ESC 0/1 h) |
| 1300 | 16 | 45 | Rule-in (ESC 0/1 h) |
| 1301 | 18 | 18 | Observation zone (0/2 h, Reichlin 2015) |
| 1302 | 15 | 16 | Rule-out (ESC 0/3 h) |
| 1303 | 5 | 4 | Rule-out (hsTnT and Copeptin 0 h, Moeckel 2014) |
| 1304 | 183 | 152 | Rule-in (ESC 0 h) |
| 1305 | 1.5 | 1.5 | Rule-out (hsTnT and Copeptin 0 h, Moeckel 2014) |
| 1306 | 15 | 15 | Observation zone (ESC 0/1 h) |
| 1307 | 67 | 71 | Rule-in (ESC 0 h) |
| 1308 | 27 | 23 | Observation zone (ESC 0/1 h) |
| 1309 | 1.5 | | Rule-out (ESC 0 h) |
| 1310 | 5 | 5 | Rule-out (ESC 0 h) |
| 1311 | 10 | 11 | Rule-out (0/2 h, Reichlin 2015) |
| 1312 | 11 | 12 | Rule-out (ESC 0 h) |
| 1313 | 9 | 7 | Rule-out (hsTnT and Copeptin 0 h, Moeckel 2014) |
| 1314 | 10 | 13 | Rule-out (ESC 0 h) |
| 1315 | 101 | 91 | Rule-in (ESC 0 h) |
| 1316 | 23 | 24 | Rule-out (ESC 0/3 h) |
| 1317 | 33 | 32 | Observation zonc (ESC 0/1 h) |
| 1318 | 5 | 5 | Rule-out (hsTnT and Copeptin 0 h, Moeckel 2014) |
| 1319 | 8 | 8 | Rule-out (hsTnT and Copeptin 0 h, Moeckel 2014) |
| 1320 | 19 | 20 | Observation zone (ESC 0/1 h) |
| 1321 | 9 | 8 | Rule-out (ESC 0/1 h) |
| 1322 | 130 | | Rule-in (ESC 0 h) |
| 1323 | 14 | 13 | Observation zonc (ESC 0/1 h) |
| 1324 | 5 | | Rule-out (ESC 0 h) |
| 1325 | 5 | 1.5 | Observation zone (ESC 0/1 h) |
| 1326 | 7 | 6 | Rule-out (ESC 0/1 h) |
| 1327 | 5 | 6 | Rule-out (ESC 0 h) |
| 1328 | 14 | 16 | Observation zone (ESC 0/1 h) |
| 1329 | 3 | 1.5 | Rule-out (ESC 0/1 h) |
| 1330 | 12 | | Rule-out (ESC 0 h) |
| 1331 | 14 | 14 | Rule-out (ESC 0 h) |
| 1332 | 23 | 26 | Rule-out (ESC 0/3 h) |
| 1333 | 5 | | Rule-out (ESC 0 h) |
| 1334 | 6 | 7 | Rule-out (ESC 0 h) |
| 1335 | 1.5 | 4 | Rule-out (ESC 0/1 h) |
| 1336 | 5 | 5 | Rule-out (0/2 h, Reichlin 2015) |
| 1337 | 630 | 844 | Rule-in (ESC 0 h) |
| 1338 | 13 | 16 | Observation zone (ESC 0/1 h) |
| 1339 | 4 | | Rule-out (hsTnT and Copeptin 0 h, Moeckel 2014) |
| 1340 | 12 | 12 | Rule-out (hsTnT and Copeptin 0 h, Moeckel 2014) |
| 1341 | 7 | 4 | Observation zone (ESC 0/1 h) |
| 1342 | 12 | 10 | Rule-out (hsTnT and Copeptin 0 h, Moeckel 2014) |
| 1343 | 1.5 | | Rule-out (ESC 0 h) |
| 1344 | 10 | 9 | Rule-out (ESC 0/1 h) |
| 1345 | 17 | 20 | Observation zonc (ESC 0/1 h) |
| 1346 | 24 | 24 | Rule-out (ESC 0/3 h) |
| 1347 | 8 | 10 | Rule-out (ESC 0/3 h) |
| 1348 | 4 | | Rule-out (ESC 0 h) |
| 1349 | 11 | 14 | Observation zone (ESC 0/1 h) |
| 1350 | 8 | 8 | Rule-out (ESC 0 h) |
| 1351 | 14 | 24 | Rule-in (ESC 0/3 h) |
| 1352 | 8 | 8 | Rule-out (ESC 0 h) |
| 1353 | 6 | 8 | Rule-out (ESC 0 h) |
| 1354 | 16 | 16 | Rule-out (ESC 0/3 h) |
| 1355 | 114 | 104 | Rule-in (ESC 0 h) |
| 1356 | 5 | 7 | Rule-out (0/2 h, Reichlin 2015) |
| 1357 | 14 | 15 | Observation zone (ESC 0/1 h) |
| 1358 | 1.5 | | Rule-out (ESC 0 h) |
| 1359 | 4 | | Rule-out (ESC 0 h) |
| 1360 | 11 | 17 | Rule-in (ESC 0/1 h) |
| 1361 | 12 | 16 | Observation zone (ESC 0/1 h) |
| 1362 | 10 | 12 | Rule-out (ESC 0 h) |
| 1363 | 25 | 25 | Observation zonc (0/2 h, Reichlin 2015) |
| 1364 | 4 | | Rule-out (ESC 0 h) |
| 1365 | 5 | 5 | Rule-out (ESC 0 h) |
| 1366 | 4 | 4 | Rule-out (ESC 0 h) |
| 1367 | 8 | 8 | Rule-out (ESC 0/1 h) |
| 1368 | 31 | 38 | Observation zone (0/2 h, Reichlin 2015) |
| 1369 | 8 | 9 | Rule-out (hsTnT and Copeptin 0 h, Moeckel 2014) |
| 1370 | 12 | 16 | Observation zone (0/2 h, Reichlin 2015) |
| 1371 | 63 | 94 | Rule-in (ESC 0 h) |
| 1372 | 10 | 12 | Rule-out (hsTnT and Copeptin 0 h, Moeckel 2014) |
| 1373 | 5 | 5 | Rule-out (hsTnT and Copoptin 0 h, Mocckel 2014) |
| 1374 | 132 | 140 | Rule-in (ESC 0 h) |
| 1375 | 5 | 5 | Rule-out (ESC 0 h) |
| 1376 | 6 | 6 | Rule-out (ESC 0 h) |
| 1377 | 732 | | Rule-in (ESC 0 h) |
| 1378 | 4 | | Rule-out (ESC 0 h) |
| 1379 | 7 | 6 | Rule-out (ESC 0/3 h) |
| 1380 | 14 | 20 | Rule-in (ESC 0/3 h) |
| 1381 | 5 | 4 | Rule-out (0/2 h, Reichlin 2015) |
| 1382 | 7 | 5 | Rule-out (ESC 0/1 h) |
| 1383 | 46 | 93 | Rule-in (ESC 0/3 h) |
| 1384 | 5 | 6 | Rule-out (hsTnT and Copeptin 0 h, Moeckel 2014) |
| 1385 | 11 | 13 | Rule-out (hsTnT and Copeptin 0 h, Moeckel 2014) |
| 1386 | 36 | 23 | Rule-in (0/2 h, Reichlin 2015) |
| 1387 | 12 | 12 | Observation zone (ESC 0/1 h) |
| 1388 | 3 | | Rule-out (ESC 0 h) |
| 1389 | 5 | 4 | Rule-out (ESC 0/3 h) |
| 1390 | 5 | 5 | Rule-out (ESC 0/1 h) |
| 1391 | 18 | 16 | Observation zonc (ESC 0/1 h) |
| 1392 | 7 | 6 | Rule-out (ESC 0/3 h) |
| 1393 | 17 | 17 | Rule-out (ESC 0/3 h) |
| 1394 | 12 | 12 | Rule-out (ESC 0/3 h) |
| 1395 | 18 | 11 | Rule-out (ESC 0/3 h) |
| 1396 | 27 | 52 | Rule-in (ESC 0/3 h) |
| 1397 | 36 | 36 | Observation zone (0/2 h, Reichlin 2015) |
| 1398 | 14 | 14 | Observation zone (ESC 0/1 h) |
| 1399 | 16 | 14 | Rule-out (ESC 0/3 h) |
| 1400 | 22 | 23 | Observation zone (0/2 h, Reichlin 2015) |
| 1401 | 6 | 7 | Rule-out (ESC 0 h) |
| 1402 | 251 | 1642 | Rule-in (ESC 0 h) |
| 1403 | 21 | 30 | Rule-in (ESC 0/1 h) |
| 1404 | 22 | 20 | Observation zone (0/2 h, Reichlin 2015) |
| 1405 | 57 | 52 | Rule-in (ESC 0 h) |
| 1406 | 11 | 11 | Rule-out (ESC 0/3 h) |
| 1407 | 9 | 9 | Rule-out (ESC 0 h) |
| 1408 | 5 | 5 | Rule-out (ESC 0 h) |
| 1409 | 9 | 9 | Rule-out (ESC 0/1 h) |
| 1410 | 9 | 8 | Rule-out (ESC 0 h) |
| 1411 | 9 | 11 | Rule-out (ESC 0/1 h) |
| 1412 | 5 | 5 | Rule-out (hsTnT and Copeptin 0 h, Moeckel 2014) |
| 1413 | 13 | 13 | Rule-out (hsTnT and Copeptin 0 h, Moeckel 2014) |
| 1414 | 7 | 7 | Rule-out (ESC 0 h) |
| 1415 | 448 | | Rule-in (ESC 0 h) |
| 1416 | 71 | 62 | Rule-in (ESC 0 h) |
| 1417 | 45 | 84 | Rule-in (ESC 0/1 h) |
| 1418 | 6 | 5 | Rule-out (ESC 0 h) |
| 1419 | 7 | 7 | Rule-out (ESC 0/1 h) |
| 1420 | 436 | | Rule-in (ESC 0 h) |
| 1421 | 34 | 39 | Rule-in (ESC 0/1 h) |
| 1422 | 8 | 7 | Rule-out (ESC 0/3 h) |
| 1423 | 44 | 69 | Rule-in (ESC 0/3 h) |
| 1424 | 4 | 4 | Rule-out (ESC 0 h) |
| 1425 | 8 | 8 | Rule-out (ESC 0/1 h) |
| 1426 | 6 | 6 | Rule-out (ESC 0/1 h) |
| 1427 | 16 | 13 | Observation zone (ESC 0/1 h) |
| 1428 | 11 | 11 | Rule-out (ESC 0 h) |
| 1429 | 10 | 11 | Rule-out (ESC 0 h) |
| 1430 | 4 | 5 | Rule-out (0/2 h, Reichlin 2015) |
| 1431 | 22 | 25 | Rule-out (ESC 0/3 h) |
| 1432 | 16 | 78 | Rule-in (ESC 0/3 h) |
| 1433 | 5 | 5 | Rule-out (hsTnT and Copeptin 0 h, Moeckel 2014) |
| 1434 | 209 | 209 | Rule-in (ESC 0 h) |
| 1435 | 15 | 17 | Rule-out (ESC 0/3 h) |
| 1436 | 7 | 9 | Rule-out (ESC 0/1 h) |
| 1437 | 29 | 33 | Observation zone (ESC 0/1 h) |
| 1438 | 10 | 10 | Rule-out (hsTnT and Copeptin 0 h, Moeckel 2014) |
| 1439 | 4 | 11 | Rule-out (ESC 0/3 h) |
| 1440 | 5 | 4 | Rule-out (ESC 0/1 h) |
| 1441 | 5 | 9 | Observation zone (ESC 0/1 h) |
| 1442 | 8 | 6 | Rule-out (ESC 0 h) |
| 1443 | 7 | 10 | Observation zone (ESC 0/1 h) |
| 1444 | 5 | 5 | Rule-out (hsTnT and Copeptin 0 h, Moeckel 2014) |
| 1445 | 7 | 7 | Rule-out (ESC 0/1 h) |
| 1446 | 5 | 4 | Rule-out (0/2 h, Reichlin 2015) |
| 1447 | 4 | | Rule-out (ESC 0 h) |
| 1448 | 35 | 31 | Observation zone (ESC 0/1 h) |
| 1449 | 17 | 18 | Observation zone (0/2 h, Reichlin 2015) |
| 1450 | 6 | 6 | Rule-out (ESC 0/1 h) |
| 1451 | 17 | 16 | Observation zone (ESC 0/1 h) |
| 1452 | 194 | 322 | Rule-in (ESC 0 h) |
| 1453 | 6 | 7 | Rule-out (ESC 0/1 h) |
| 1454 | 7 | 7 | Rule-out (hsTnT and Copeptin 0 h, Moeckel 2014) |
| 1455 | 4 | | Rule-out (ESC 0 h) |
| 1456 | 12 | 18 | Rule-in (ESC 0/1 h) |
| 1457 | 246 | 239 | Rule-in (ESC 0 h) |
| 1458 | 4 | 3 | Rule-out (0/2 h, Reichlin 2015) |
| 1459 | 5 | 4 | Rule-out (ESC 0 h) |
| 1460 | 6 | 5 | Rule-out (ESC 0/3 h) |
| 1461 | 5 | 5 | Rule-out (0/2 h, Reichlin 2015) |
| 1462 | 17 | 17 | Rule-out (ESC 0/3 h) |
| 1463 | 29 | 40 | Rule-in (ESC 0/1 h) |
| 1464 | 6 | 5 | Rule-out (hsTnT and Copeptin 0 h, Moeckel 2014) |
| 1465 | 11 | 12 | Rule-out (ESC 0/1 h) |
| 1466 | 3 | 4 | Rule-out (ESC 0/1 h) |
| 1467 | 24 | 24 | Observation zone (ESC 0/1 h) |
| 1468 | 10 | 18 | Observation zone (0/2 h, Reichlin 2015) |
| 1469 | 8 | 8 | Rule-out (0/2 h, Reichlin 2015) |
| 1470 | 20 | 21 | Observation zone (0/2 h, Reichlin 2015) |
| 1471 | 111 | 101 | Rule-in (ESC 0 h) |
| 1472 | 11 | 10 | Rule-out (0/2 h, Reichlin 2015) |
| 1473 | 56 | | Rule-in (ESC 0 h) |
| 1474 | 11 | 9 | Rule-out (ESC 0 h) |
| 1475 | 16 | 15 | Observation zonc (ESC 0/1 h) |
| 1476 | 6 | 6 | Rule-out (ESC 0/3 h) |
| 1477 | 9 | 10 | Rule-out (0/2 h, Reichlin 2015) |
| 1478 | 11 | 11 | Rule-out (0/2 h, Reichlin 2015) |
| 1479 | 34 | 32 | Observation zone (0/2 h, Reichlin 2015) |
| 1480 | 3 | 4 | Rule-out (ESC 0 h) |
| 1481 | 39 | 140 | Rule-in (0/2 h, Reichlin 2015) |
| 1482 | 7 | 8 | Rule-out (ESC 0 h) |
| 1483 | 9 | 9 | Rule-out (ESC 0/1 h) |
| 1484 | 6 | 5 | Rule-out (ESC 0 h) |
| 1485 | 5 | | Rule-out (ESC 0 h) |
| 1486 | 147 | 145 | Rule-in (ESC 0 h) |
| 1487 | 4 | 5 | Rule-out (hsTnT and Copeptin 0 h, Moeckel 2014) |
| 1488 | 7 | 7 | Rule-out (hsTnT and Copeptin 0 h, Moeckel 2014) |
| 1489 | 62 | 63 | Rule-in (ESC 0 h) |
| 1490 | 8 | 6 | Rule-out (ESC 0/1 h) |
| 1491 | 18 | 73 | Rule-in (ESC 0/3 h) |
| 1492 | 157 | 684 | Rule-in (ESC 0 h) |
| 1493 | 29 | 32 | Observation zone (0/2 h, Reichlin 2015) |
| 1494 | 6 | 5 | Rule-out (hsTnT and Copeptin 0 h, Moeckel 2014) |
| 1495 | 17 | 15 | Observation zone (ESC 0/1 h) |
| 1496 | 30 | 23 | Rule-in (ESC 0/3 h) |
| 1497 | 7 | 8 | Rule-out (hsTnT and Copeptin 0 h, Moeckel 2014) |
| 1498 | 11 | 8 | Observation zone (ESC 0/1 h) |
| 1499 | 10 | 9 | Rule-out (hsTnT and Copeptin 0 h, Moeckel 2014) |
| 1500 | 4 | | Rule-out (ESC 0 h) |
| 1501 | 10 | 10 | Rule-out (ESC 0/3 h) |
| 1502 | 12 | 14 | Observation zone (ESC 0/1 h) |
| 1503 | 6 | 4 | Rule-out (ESC 0/3 h) |
| 1504 | 9 | 10 | Rule-out (ESC 0 h) |
| 1505 | 37 | 31 | Rule-out (ESC 0/3 h) |
| 1506 | 72 | 68 | Rule-in (ESC 0 h) |
| 1507 | 54 | 61 | Rule-in (ESC 0 h) |
| 1508 | 6 | 6 | Rule-out (ESC 0/1 h) |
| 1509 | 8 | 8 | Rule-out (ESC 0/1 h) |
| 1510 | 7 | 6 | Rule-out (0/2 h, Reichlin 2015) |
| 1511 | 14 | 14 | Observation zone (ESC 0/1 h) |
| 1512 | 6 | 6 | Rule-out (ESC 0 h) |
| 1513 | 5 | 4 | Rule-out (ESC 0/1 h) |
| 1514 | 83 | 81 | Rule-in (ESC 0 h) |
| 1515 | 23 | 26 | Rule-out (ESC 0/3 h) |
| 1516 | 11 | 12 | Rule-out (ESC 0 h) |
| 1517 | 269 | 298 | Rule-in (ESC 0 h) |
| 1518 | 48 | 46 | Rule-out (ESC 0/3 h) |
| 1519 | 19 | 20 | Rule-out (ESC 0/3 h) |
| 1520 | 6 | 5 | Rule-out (0/2 h, Reichlin 2015) |
| 1521 | 4 | 5 | Rule-out (ESC 0/1 h) |
| 1522 | 14 | 14 | Rule-out (ESC 0/3 h) |
| 1523 | 6 | 6 | Rule-out (0/2 h, Reichlin 2015) |
| 1524 | 4 | | Rule-out (ESC 0 h) |
| 1525 | 10 | 8 | Rule-out (ESC 0 h) |
| 1526 | 19 | 6368 | Rule-in (ESC 0/3 h) |
| 1527 | 143 | 183 | Rule-in (ESC 0 h) |
| 1528 | 23 | 96 | Rule-in (ESC 0/3 h) |
| 1529 | 54 | 53 | Rule-in (ESC 0 h) |
| 1530 | 7 | 8 | Rule-out (hsTnT and Copeptin 0 h, Moeckel 2014) |
| 1531 | 13 | 11 | Rule-out (hsTnT and Copcptin 0 h, Mocckel 2014) |
| 1532 | 5 | 5 | Rule-out (0/2 h, Reichlin 2015) |
| 1533 | 15 | 15 | Observation zone (0/2 h, Reichlin 2015) |
| 1534 | 6 | 6 | Rule-out (0/2 h, Reichlin 2015) |
| 1535 | 11 | 9 | Rule-out (ESC 0 h) |
| 1536 | 8 | 8 | Rule-out (ESC 0 h) |
| 1537 | 10 | 10 | Rule-out (ESC 0/3 h) |
| 1538 | 13 | 13 | Rule-out (0/2 h, Reichlin 2015) |
| 1539 | 4 | 6 | Rule-out (ESC 0 h) |
| 1540 | 271 | 243 | Rule-in (ESC 0 h) |
| 1541 | 9 | 9 | Rule-out (ESC 0 h) |
| 1542 | 16 | 17 | Observation zone (0/2 h, Reichlin 2015) |
| 1543 | 26 | 23 | Observation zone (ESC 0/1 h) |
| 1544 | 22 | 30 | Rule-in (ESC 0/1 h) |
| 1545 | 174 | 217 | Rule-in (ESC 0 h) |
| 1546 | 6 | 5 | Rule-out (ESC 0/1 h) |
| 1547 | 4 | | Rule-out (ESC 0 h) |
| 1548 | 23 | 21 | Observation zone (0/2 h, Reichlin 2015) |
| 1549 | 8 | 7 | Rule-out (ESC 0 h) |
| 1550 | 5 | | Rule-out (ESC 0 h) |
| 1551 | 7 | 6 | Rule-out (ESC 0 h) |
| 1552 | 24 | 22 | Observation zone (0/2 h, Reichlin 2015) |
| 1553 | 16 | 16 | Observation zone (ESC 0/1 h) |
| 1554 | 21 | 18 | Observation zone (0/2 h, Reichlin 2015) |
| 1555 | 44 | 32 | Rule-in (ESC 0/1 h) |
| 1556 | 7 | 6 | Rule-out (ESC 0 h) |
| 1557 | 47 | 50 | Rule-out (ESC 0/3 h) |
| 1558 | 18 | 14 | Observation zone (0/2 h, Reichlin 2015) |
| 1559 | 163 | 151 | Rule-in (ESC 0 h) |
| 1560 | 59 | 76 | Rule-in (ESC 0 h) |
| 1561 | 20 | 23 | Rule-in (ESC 0/3 h) |
| 1562 | 6 | 8 | Rule-out (ESC 0/3 h) |
| 1563 | 10 | 16 | Rule-in (ESC 0/1 h) |
| 1564 | 14 | 14 | Observation zone (ESC 0/1 h) |
| 1565 | 415 | 500 | Rule-in (ESC 0 h) |
| 1566 | 6 | 6 | Rule-out (ESC 0/1 h) |
| 1567 | 21 | 30 | Rule-in (ESC 0/3 h) |
| 1568 | 20 | 23 | Rule-out (ESC 0/3 h) |
| 1569 | 6 | 8 | Rule-out (ESC 0/3 h) |
| 1570 | 7 | 7 | Rule-out (hsTnT and Copeptin 0 h, Moeckel 2014) |
| 1571 | 13 | 16 | Observation zone (ESC 0/1 h) |
| 1572 | 92 | 77 | Rule-in (ESC 0 h) |
| 1573 | 8 | 8 | Rule-out (ESC 0 h) |
| 1574 | 5 | 5 | Rule-out (ESC 0 h) |
| 1575 | 13 | 11 | Rule-out (0/2 h, Reichlin 2015) |
| 1576 | 10 | 12 | Rule-out (ESC 0 h) |
| 1577 | 25 | 29 | Observation zone (0/2 h, Reichlin 2015) |
| 1578 | 13 | 13 | Observation zone (ESC 0/1 h) |
| 1579 | 26 | 24 | Rule-out (ESC 0/3 h) |
| 1580 | 8 | 8 | Rule-out (hsTnT and Copeptin 0 h, Moeckel 2014) |
| 1581 | 8 | 9 | Rule-out (ESC 0 h) |
| 1582 | 5 | 5 | Rule-out (0/2 h, Reichlin 2015) |
| 1583 | 102 | 108 | Rule-in (ESC 0 h) |
| 1584 | 20 | 19 | Observation zone (0/2 h, Reichlin 2015) |
| 1585 | 8 | 9 | Rule-out (0/2 h, Reichlin 2015) |
| 1586 | 6 | 16 | Rule-in (ESC 0/1 h) |
| 1587 | 11 | 10 | Rule-out (ESC 0/1 h) |
| 1588 | 5 | 4 | Rule-out (ESC 0 h) |
| 1589 | 3 | | Rule-out (hsTnT and Copeptin 0 h, Moeckel 2014) |
| 1590 | 15 | 16 | Rule-out (ESC 0/3 h) |
| 1591 | 19 | 26 | Rule-in (ESC 0/3 h) |
| 1592 | 5 | 4 | Rule-out (ESC 0/1 h) |
| 1593 | 6 | 5 | Rule-out (hsTnT and Copeptin 0 h, Moeckel 2014) |
| 1594 | 6 | | Rule-out (ESC 0 h) |
| 1595 | 5 | | Rule-out (hsTnT and Copeptin 0 h, Moeckel 2014) |
| 1596 | 8 | 7 | Rule-out (ESC 0/3 h) |
| 1597 | 7 | 4 | Observation zonc (ESC 0/1 h) |
| 1598 | 4 | 6 | Rule-out (ESC 0 h) |
| 1599 | 16 | 15 | Observation zone (ESC 0/1 h) |
| 1600 | 20 | 21 | Observation zone (ESC 0/1 h) |
| 1601 | 107 | 110 | Rule-in (ESC 0 h) |
| 1602 | 25 | 22 | Rule-in (ESC 0/3 h) |
| 1603 | 15 | 14 | Observation zonc (ESC 0/1 h) |
| 1604 | 6 | 7 | Rule-out (ESC 0/1 h) |
| 1605 | 5 | 6 | Rule-out (0/2 h, Reichlin 2015) |
| 1606 | 5 | 5 | Rule-out (0/2 h, Reichlin 2015) |
| 1607 | 7 | 7 | Rule-out (ESC 0/1 h) |
| 1608 | 7 | 6 | Rule-out (ESC 0/1 h) |
| 1609 | 11 | 9 | Rule-out (ESC 0 h) |
| 1610 | 10 | 8 | Rule-out (ESC 0 h) |
| 1611 | 14 | 15 | Rule-out (ESC 0/3 h) |
| 1612 | 8 | 9 | Rule-out (0/2 h, Reichlin 2015) |
| 1613 | 32 | 30 | Observation zone (0/2 h, Reichlin 2015) |
| 1614 | 13 | 14 | Rule-out (ESC 0/3 h) |
| 1615 | 13 | 16 | Rule-out (ESC 0 h) |
| 1616 | 9 | 9 | Rule-out (ESC 0/3 h) |
| 1617 | 6 | 8 | Rule-out (0/2 h, Reichlin 2015) |
| 1618 | 14 | 12 | Observation zone (0/2 h, Reichlin 2015) |
| 1619 | 52 | 58 | Rule-in (ESC 0 h) |
| 1620 | 10 | 9 | Rule-out (ESC 0 h) |
| 1621 | 6 | 5 | Rule-out (ESC 0/1 h) |
| 1622 | 10 | 9 | Rule-out (ESC 0/1 h) |
| 1623 | 124 | 99 | Rule-in (ESC 0 h) |
| 1624 | 11 | 10 | Rule-out (ESC 0/1 h) |
| 1625 | 11 | 12 | Rule-out (ESC 0/1 h) |
| 1626 | 397 | 384 | Rule-in (ESC 0 h) |
| 1627 | 29 | 34 | Rule-out (ESC 0/3 h) |
| 1628 | 6 | 6 | Rule-out (ESC 0 h) |
| 1629 | 81 | 89 | Rule-in (ESC 0 h) |
| 1630 | 241 | | Rule-in (ESC 0 h) |
| 1631 | 84 | 85 | Rule-in (ESC 0 h) |
| 1632 | 53 | 84 | Rule-in (ESC 0 h) |
| 1633 | 39 | 47 | Rule-in (ESC 0/1 h) |
| 1634 | 20 | 19 | Rule-out (ESC 0/3 h) |
| 1635 | 7 | 6 | Rule-out (ESC 0 h) |
| 1636 | 5 | 5 | Rule-out (0/2 h, Reichlin 2015) |
| 1637 | 13 | 14 | Observation zone (ESC 0/1 h) |
| 1638 | 16 | 15 | Observation zone (ESC 0/1 h) |
| 1639 | 10 | 11 | Rule-out (0/2 h, Reichlin 2015) |
| 1640 | 4 | | Rule-out (ESC 0 h) |
| 1641 | 5 | 7 | Rule-out (ESC 0/1 h) |
| 1642 | 6 | 6 | Rule-out (hsTnT and Copeptin 0 h, Moeckel 2014) |
| 1643 | 333 | 383 | Rule-in (ESC 0 h) |
| 1644 | 197 | 161 | Rule-in (ESC 0 h) |
| 1645 | 31 | 27 | Observation zone (ESC 0/1 h) |
| 1646 | 5 | 5 | Rule-out (ESC 0/1 h) |
| 1647 | 11 | 13 | Rule-out (ESC 0 h) |
| 1648 | 7 | 15 | Rule-in (ESC 0/3 h) |
| 1649 | 7 | 8 | Rule-out (0/2 h, Reichlin 2015) |
| 1650 | 14 | 15 | Rule-out (ESC 0/3 h) |
| 1651 | 12 | 12 | Observation zone (ESC 0/1 h) |
| 1652 | 26 | 57 | Rule-in (ESC 0/3 h) |
| 1653 | 21 | 18 | Observation zonc (ESC 0/1 h) |
| 1654 | 64 | 78 | Rule-in (ESC 0 h) |
| 1655 | 74 | 67 | Rule-in (ESC 0 h) |
| 1656 | 1.5 | | Rule-out (ESC 0 h) |
| 1657 | 10 | 10 | Rule-out (0/2 h, Reichlin 2015) |
| 1658 | 11 | 12 | Rule-out (0/2 h, Reichlin 2015) |
| 1659 | 6 | 7 | Rule-out (ESC 0/1 h) |
| 1660 | 26 | 26 | Observation zone (0/2 h, Reichlin 2015) |
| 1661 | 45 | 105 | Rule-in (0/2 h, Reichlin 2015) |
| 1662 | 18 | 17 | Observation zone (ESC 0/1 h) |
| 1663 | 11 | 11 | Rule-out (ESC 0/3 h) |
| 1664 | 18 | 14 | Rule-out (ESC 0/3 h) |
| 1665 | 4 | 4 | Rule-out (ESC 0/3 h) |
| 1666 | 12 | 13 | Rule-out (ESC 0 h) |
| 1667 | 9 | 8 | Rule-out (ESC 0/1 h) |
| 1668 | 16 | 15 | Rule-out (ESC 0/3 h) |
| 1669 | 34 | 25 | Rule-in (ESC 0/3 h) |
| 1670 | 5 | 7 | Rule-out (ESC 0 h) |
| 1671 | 6 | 7 | Rule-out (0/2 h, Reichlin 2015) |
| 1672 | 5 | 4 | Rule-out (ESC 0/1 h) |
| 1673 | 8 | 7 | Rule-out (ESC 0/1 h) |
| 1674 | 10 | 9 | Rule-out (0/2 h, Reichlin 2015) |
| 1675 | 15 | 16 | Observation zone (0/2 h, Reichlin 2015) |
| 1676 | 24 | 27 | Observation zone (ESC 0/1 h) |
| 1677 | 6 | 8 | Rule-out (ESC 0/3 h) |
| 1678 | 45 | 46 | Rule-out (ESC 0/3 h) |
| 1679 | 5 | 5 | Rule-out (ESC 0/3 h) |
| 1680 | 11 | 14 | Observation zone (ESC 0/1 h) |
| 1681 | 18 | 17 | Observation zone (0/2 h, Reichlin 2015) |
| 1682 | 25 | 24 | Rule-out (ESC 0/3 h) |
| 1683 | 38 | 36 | Observation zone (0/2 h, Reichlin 2015) |
| 1684 | 85 | 92 | Rule-in (ESC 0 h) |
| 1685 | 7 | 6 | Rule-out (0/2 h, Reichlin 2015) |
| 1686 | 8 | 7 | Rule-out (ESC 0 h) |
| 1687 | 6 | 5 | Rule-out (ESC 0/1 h) |
| 1688 | 1.5 | 5 | Observation zone (ESC 0/1 h) |
| 1689 | 21 | 19 | Observation zone (ESC 0/1 h) |
| 1690 | 24 | 21 | Observation zone (0/2 h, Reichlin 2015) |
| 1691 | 1.5 | | Rule-out (ESC 0 h) |
| 1692 | 20 | 71 | Rule-in (ESC 0/3 h) |
| 1693 | 6 | 7 | Rule-out (0/2 h, Reichlin 2015) |
| 1694 | 21 | 20 | Observation zone (ESC 0/1 h) |
| 1695 | 7 | 8 | Rule-out (ESC 0/1 h) |
| 1696 | 11 | 10 | Rule-out (0/2 h, Reichlin 2015) |
| 1697 | 7 | 6 | Rule-out (ESC 0/1 h) |
| 1698 | 9 | 8 | Rule-out (hsTnT and Copeptin 0 h, Moeckel 2014) |
| 1699 | 46 | 49 | Observation zone (0/2 h, Reichlin 2015) |
| 1700 | 4 | 5 | Rule-out (0/2 h, Reichlin 2015) |
| 1701 | 79 | 83 | Rule-in (ESC 0 h) |
| 1702 | 86 | 2323 | Rule-in (ESC 0 h) |
| 1703 | 38 | 35 | Observation zone (0/2 h, Reichlin 2015) |
| 1704 | 8 | 8 | Rule-out (ESC 0 h) |
| 1705 | 112 | 136 | Rule-in (ESC 0 h) |
| 1706 | 5 | 4 | Rule-out (ESC 0/1 h) |
| 1707 | 9 | 7 | Rule-out (ESC 0 h) |
| 1708 | 173 | 151 | Rule-in (ESC 0 h) |
| 1709 | 9 | 7 | Rule-out (ESC 0 h) |
| 1710 | 28 | 29 | Rule-out (ESC 0/3 h) |
| 1711 | 1.5 | 1.5 | Rule-out (0/2 h, Reichlin 2015) |
| 1712 | 6 | 5 | Rule-out (ESC 0/1 h) |
| 1713 | 6 | 7 | Rule-out (hsTnT and Copeptin 0 h, Moeckel 2014) |
| 1714 | 1.5 | 3 | Rule-out (hsTnT and Copeptin 0 h, Moeckel 2014) |
| 1715 | 26 | 25 | Observation zonc (ESC 0/1 h) |
| 1716 | 173 | 173 | Rule-in (ESC 0 h) |
| 1717 | 830 | 682 | Rule-in (ESC 0 h) |
| 1718 | 1.5 | | Rule-out (ESC 0 h) |
| 1719 | 6 | 6 | Rule-out (ESC 0/1 h) |
| 1720 | 6 | 7 | Rule-out (ESC 0/1 h) |
| 1721 | 22 | 20 | Observation zone (ESC 0/1 h) |
| 1722 | 12 | 12 | Observation zone (ESC 0/1 h) |
| 1723 | 22 | 22 | Observation zone (ESC 0/1 h) |
| 1724 | 4 | 4 | Rule-out (ESC 0 h) |
| 1725 | 63 | 59 | Rule-in (ESC 0 h) |
| 1726 | 8 | 7 | Rule-out (ESC 0/1 h) |
| 1727 | 7 | 8 | Rule-out (ESC 0/1 h) |
| 1728 | 361 | | Rule-in (ESC 0 h) |
| 1729 | 8 | 9 | Rule-out (ESC 0/1 h) |
| 1730 | 6 | 6 | Rule-out (ESC 0/1 h) |
| 1731 | 40 | 39 | Rule-out (ESC 0/3 h) |
| 1732 | 5 | 7 | Rule-out (hsTnT and Copeptin 0 h, Moeckel 2014) |
| 1733 | 32 | 59 | Rule-in (ESC 0/3 h) |
| 1734 | 6 | 6 | Rule-out (hsTnT and Copeptin 0 h, Moeckel 2014) |
| 1735 | 4 | 1.5 | Rule-out (ESC 0 h) |
| 1736 | 6 | 7 | Rule-out (ESC 0 h) |
| 1737 | 5 | 5 | Rule-out (ESC 0 h) |
| 1738 | 20 | 19 | Observation zone (ESC 0/1 h) |
| 1739 | 26 | 52 | Rule-in (ESC 0/1 h) |
| 1740 | 6 | 6 | Rule-out (ESC 0 h) |
| 1741 | 32 | 83 | Rule-in (ESC 0/3 h) |
| 1742 | 10 | 13 | Rule-in (ESC 0/3 h) |
| 1743 | 5 | 1.5 | Observation zonc (ESC 0/1 h) |
| 1744 | 5 | 6 | Rule-out (ESC 0/1 h) |
| 1745 | 7 | 6 | Rule-out (ESC 0 h) |
| 1746 | 4 | 4 | Rule-out (0/2 h, Reichlin 2015) |
| 1747 | 5 | 7 | Rule-out (ESC 0/1 h) |
| 1748 | 5 | 4 | Rule-out (0/2 h, Reichlin 2015) |
| 1749 | 6 | 6 | Rule-out (hsTnT and Copeptin 0 h, Moeckel 2014) |
| 1750 | 11 | 12 | Rule-out (0/2 h, Reichlin 2015) |
| 1751 | 6 | 5 | Rule-out (ESC 0 h) |
| 1752 | 5 | | Rule-out (ESC 0 h) |
| 1753 | 7 | 7 | Rule-out (ESC 0 h) |
| 1754 | 26 | 33 | Rule-in (ESC 0/3 h) |
| 1755 | 8 | 7 | Rule-out (ESC 0 h) |
| 1756 | 253 | | Rule-in (ESC 0 h) |
| 1757 | 4 | 5 | Rule-out (ESC 0 h) |
| 1758 | 9 | 10 | Rule-out (ESC 0 h) |
| 1759 | 10 | 10 | Rule-out (ESC 0 h) |
| 1760 | 5 | 6 | Rule-out (hsTnT and Copeptin 0 h, Moeckel 2014) |
| 1761 | 6 | 7 | Rule-out (hsTnT and Copeptin 0 h, Moeckel 2014) |
| 1762 | 5 | 6 | Rule-out (0/2 h, Reichlin 2015) |
| 1763 | 4 | | Rule-out (ESC 0 h) |
| 1764 | 8 | 7 | Rule-out (ESC 0 h) |
| 1765 | 6 | 6 | Rule-out (0/2 h, Reichlin 2015) |
| 1766 | 4 | 4 | Rule-out (ESC 0/1 h) |
| 1767 | 6 | 8 | Rule-out (ESC 0 h) |
| 1768 | 6 | 6 | Rule-out (ESC 0 h) |
| 1769 | 7 | 6 | Rule-out (ESC 0 h) |
| 1770 | 6 | 5 | Rule-out (ESC 0/1 h) |
| 1771 | 6 | 6 | Rule-out (ESC 0/1 h) |
| 1772 | 10 | 10 | Rule-out (ESC 0/1 h) |
| 1773 | 33 | 36 | Observation zone (0/2 h, Reichlin 2015) |
| 1774 | 767 | 719 | Rule-in (ESC 0 h) |
| 1775 | 7 | 6 | Rule-out (ESC 0 h) |
| 1776 | 31 | 34 | Observation zone (ESC 0/1 h) |
| 1777 | 7 | 5 | Rule-out (ESC 0 h) |
| 1778 | 8 | 9 | Rule-out (0/2 h, Reichlin 2015) |
| 1779 | 4 | 5 | Rule-out (ESC 0 h) |
| 1780 | 8 | 9 | Rule-out (0/2 h, Reichlin 2015) |
| 1781 | 6 | 7 | Rule-out (ESC 0 h) |
| 1782 | 10 | 9 | Rule-out (0/2 h, Reichlin 2015) |
| 1783 | 9 | 10 | Rule-out (0/2 h, Reichlin 2015) |
| 1784 | 18 | 16 | Observation zone (ESC 0/1 h) |
| 1785 | 24 | 23 | Rule-out (ESC 0/3 h) |
| 1786 | 7 | 7 | Rule-out (ESC 0 h) |
| 1787 | 75 | 63 | Rule-in (ESC 0 h) |
| 1788 | 14 | 14 | Observation zone (ESC 0/1 h) |
| 1789 | 14 | 13 | Observation zone (0/2 h, Reichlin 2015) |
| 1790 | 6 | 4 | Rule-out (0/2 h, Reichlin 2015) |
| 1791 | 47 | 47 | Observation zone (0/2 h, Reichlin 2015) |
| 1792 | 6 | 7 | Rule-out (0/2 h, Reichlin 2015) |
| 1793 | 5 | | Rule-out (ESC 0 h) |
| 1794 | 20 | 20 | Observation zone (0/2 h, Reichlin 2015) |
| 1795 | 67 | 67 | Rule-in (ESC 0 h) |
| 1796 | 540 | 558 | Rule-in (ESC 0 h) |
| 1797 | 938 | 873 | Rule-in (ESC 0 h) |
| 1798 | 8 | 5 | Rule-out (0/2 h, Reichlin 2015) |
| 1799 | 8 | 5 | Observation zonc (ESC 0/1 h) |
| 1800 | 42 | 43 | Observation zone (ESC 0/1 h) |
| 1801 | 100 | 187 | Rule-in (ESC 0 h) |
| 1802 | 11 | 6 | Rule-in (ESC 0/1 h) |
| 1803 | 48 | 45 | Observation zone (ESC 0/1 h) |
| 1804 | 6 | 6 | Rule-out (ESC 0 h) |
| 1805 | 4 | 6 | Rule-out (ESC 0 h) |
| 1806 | 18 | 21 | Observation zone (0/2 h, Reichlin 2015) |
| 1807 | 5 | 1.5 | Rule-out (0/2 h, Reichlin 2015) |
| 1808 | 6 | 5 | Rule-out (ESC 0/3 h) |
| 1809 | 6 | 5 | Rule-out (ESC 0/1 h) |
| 1810 | 3 | 7 | Observation zone (ESC 0/1 h) |
| 1811 | 1354 | 1311 | Rule-in (ESC 0 h) |
| 1812 | 16 | 19 | Observation zone (ESC 0/1 h) |
| 1813 | 7 | 10 | Observation zone (ESC 0/1 h) |
| 1814 | 3 | 1.5 | Rule-out (ESC 0/1 h) |
| 1815 | 3 | 5 | Rule-out (ESC 0 h) |
| 1816 | 4 | 1.5 | Rule-out (ESC 0/1 h) |
| 1817 | 14 | 12 | Observation zone (ESC 0/1 h) |
| 1818 | 11 | 5 | Rule-in (ESC 0/1 h) |
| 1819 | 7 | 12 | Observation zone (0/2 h, Reichlin 2015) |
| 1820 | 6 | 10 | Observation zone (ESC 0/1 h) |
| 1821 | 118 | 134 | Rule-in (ESC 0 h) |
| 1822 | 1.5 | | Rule-out (ESC 0 h) |
| 1823 | 11 | 8 | Observation zone (ESC 0/1 h) |
| 1824 | 8 | 8 | Rule-out (ESC 0/3 h) |
| 1825 | 3 | 4 | Rule-out (ESC 0/1 h) |
| 1826 | 24 | 19 | Rule-in (ESC 0/1 h) |
| 1827 | 5 | 1.5 | Rule-out (0/2 h, Reichlin 2015) |
| 1828 | 3 | | Rule-out (ESC 0 h) |
| 1829 | 20 | 25 | Observation zone (0/2 h, Reichlin 2015) |
| 1830 | 9 | 9 | Rule-out (ESC 0/1 h) |
| 1831 | 41 | 39 | Observation zone (ESC 0/1 h) |
| 1832 | 11 | 45 | Rule-in (0/2 h, Reichlin 2015) |
| 1833 | 14 | 14 | Observation zone (0/2 h, Reichlin 2015) |
| 1834 | 10 | 8 | Rule-out (ESC 0/1 h) |
| 1835 | 8 | 8 | Rule-out (ESC 0/1 h) |
| 1836 | 98 | 164 | Rule-in (ESC 0 h) |
| 1837 | 29 | 92 | Rule-in (ESC 0/1 h) |
| 1838 | 6 | 5 | Rule-out (0/2 h, Reichlin 2015) |
| 1839 | 23 | 20 | Observation zonc (ESC 0/1 h) |
| 1840 | 7 | 10 | Observation zone (ESC 0/1 h) |
| 1841 | 11 | 8 | Rule-out (0/2 h, Reichlin 2015) |
| 1842 | 11 | 12 | Rule-out (ESC 0/1 h) |
| 1843 | 14 | 10 | Observation zone (ESC 0/1 h) |
| 1844 | 32 | 26 | Rule-out (ESC 0/3 h) |
| 1845 | 6 | 6 | Rule-out (ESC 0/1 h) |
| 1846 | 196 | 265 | Rule-in (ESC 0 h) |
| 1847 | 5 | 7 | Rule-out (ESC 0/1 h) |
| 1848 | 296 | 464 | Rule-in (ESC 0 h) |
| 1849 | 21 | 20 | Observation zonc (ESC 0/1 h) |
| 1850 | 46 | 44 | Observation zone (0/2 h, Reichlin 2015) |
| 1851 | 6 | 12 | Rule-in (ESC 0/1 h) |
| 1852 | 71 | 90 | Rule-in (ESC 0 h) |
| 1853 | 8 | 8 | Rule-out (ESC 0/1 h) |
| 1854 | 7 | 7 | Rule-out (ESC 0 h) |
| 1855 | 6 | 5 | Rule-out (ESC 0/1 h) |
| 1856 | 5 | 4 | Rule-out (hsTnT and Copeptin 0 h, Moeckel 2014) |
| 1857 | 5 | 5 | Rule-out (hsTnT and Copeptin 0 h, Moeckel 2014) |
| 1858 | 16 | 18 | Observation zone (ESC 0/1 h) |
| 1859 | 348 | 375 | Rule-in (ESC 0 h) |
| 1860 | 15 | 16 | Observation zone (ESC 0/1 h) |
| 1861 | 4 | 1.5 | Rule-out (ESC 0 h) |
| 1862 | 32 | 40 | Observation zone (0/2 h, Reichlin 2015) |
| 1863 | 1638 | | Rule-in (ESC 0 h) |
| 1864 | 30 | 30 | Observation zone (ESC 0/1 h) |
| 1865 | 31 | 28 | Observation zone (ESC 0/1 h) |
| 1866 | 2776 | | Rule-in (ESC 0 h) |
| 1867 | 6 | 6 | Rule-out (ESC 0 h) |
| 1868 | 11 | 10 | Rule-out (ESC 0/1 h) |
| 1869 | 7 | 6 | Rule-out (ESC 0/1 h) |
| 1870 | 9 | 10 | Rule-out (0/2 h, Reichlin 2015) |
| 1871 | 15 | 19 | Observation zone (0/2 h, Reichlin 2015) |
| 1872 | 60 | 64 | Rule-in (ESC 0 h) |
| 1873 | 3 | 1.5 | Rule-out (0/2 h, Reichlin 2015) |
| 1874 | 11 | 14 | Observation zone (ESC 0/1 h) |
| 1875 | 6 | 7 | Rule-out (ESC 0/1 h) |
| 1876 | 5 | 1.5 | Observation zone (ESC 0/1 h) |
| 1877 | 28 | 27 | Observation zonc (ESC 0/1 h) |
| 1878 | 1.5 | | Rule-out (ESC 0 h) |
| 1879 | 8 | 4 | Rule-out (ESC 0 h) |
| 1880 | 8 | 5 | Rule-out (0/2 h, Reichlin 2015) |
| 1881 | 7 | 4 | Observation zone (ESC 0/1 h) |
| 1882 | 12 | 19 | Rule-out (ESC 0/3 h) |
| 1883 | 16 | 15 | Observation zonc (0/2 h, Reichlin 2015) |
| 1884 | 10 | 11 | Rule-out (ESC 0 h) |
| 1885 | 1.5 | 4 | Rule-out (ESC 0 h) |
| 1886 | 1.5 | 4 | Rule-out (ESC 0 h) |
| 1887 | 4 | 5 | Rule-out (ESC 0 h) |
| 1888 | 1.5 | 1.5 | Rule-out (ESC 0 h) |
| 1889 | 8 | | Rule-out (ESC 0 h) |
| 1890 | 7 | 7 | Rule-out (ESC 0/1 h) |
| 1891 | 593 | 803 | Rule-in (ESC 0 h) |
| 1892 | 8 | 7 | Rule-out (ESC 0 h) |
| 1893 | 504 | 497 | Rule-in (ESC 0 h) |
| 1894 | 4 | 5 | Rule-out (ESC 0 h) |
| 1895 | 22 | 22 | Rule-out (ESC 0/3 h) |
| 1896 | 5 | 6 | Rule-out (ESC 0 h) |
| 1897 | 21 | 21 | Observation zone (ESC 0/1 h) |
| 1898 | 18 | 22 | Rule-in (ESC 0/3 h) |
| 1899 | 9 | 11 | Rule-out (0/2 h, Reichlin 2015) |
| 1900 | 7 | 9 | Rule-out (ESC 0 h) |
| 1901 | 7 | 8 | Rule-out (0/2 h, Reichlin 2015) |
| 1902 | 214 | 463 | Rule-in (ESC 0 h) |
| 1903 | 28 | 22 | Rule-in (ESC 0/1 h) |
| 1904 | 243 | 272 | Rule-in (ESC 0 h) |
| 1905 | 7 | 7 | Rule-out (ESC 0/1 h) |
| 1906 | 20 | 21 | Observation zone (ESC 0/1 h) |
| 1907 | 7 | 16 | Rule-in (ESC 0/3 h) |
| 1908 | 5 | 7 | Rule-out (hsTnT and Copeptin 0 h, Moeckel 2014) |
| 1909 | 4 | | Rule-out (hsTnT and Copeptin 0 h, Moeckel 2014) |
| 1910 | 16 | 10 | Rule-in (ESC 0/1 h) |
| 1911 | 5 | 1.5 | Rule-out (0/2 h, Reichlin 2015) |
| 1912 | 1.5 | 1.5 | Rule-out (0/2 h, Reichlin 2015) |
| 1913 | 12 | 9 | Observation zone (ESC 0/1 h) |
| 1914 | 1.5 | 1.5 | Rule-out (ESC 0/3 h) |
| 1915 | 13 | 12 | Rule-out (hsTnT and Copeptin 0 h, Moeckel 2014) |
| 1916 | 36 | 42 | Observation zone (0/2 h, Reichlin 2015) |
| 1917 | 9 | 8 | Rule-out (ESC 0/1 h) |
| 1918 | 4 | 4 | Rule-out (ESC 0 h) |
| 1919 | 27 | 24 | Observation zone (ESC 0/1 h) |
| 1920 | 5 | 1.5 | Observation zone (ESC 0/1 h) |
| 1921 | 6 | 3 | Rule-out (0/2 h, Reichlin 2015) |
| 1922 | 7 | 5 | Rule-out (ESC 0 h) |
| 1923 | 5 | 7 | Rule-out (ESC 0/1 h) |
| 1924 | 31 | 31 | Rule-out (ESC 0/3 h) |
| 1925 | 592 | 800 | Rule-in (ESC 0 h) |
| 1926 | 8 | 11 | Rule-out (ESC 0/3 h) |
| 1927 | 1.5 | | Rule-out (ESC 0 h) |
| 1928 | 6 | 9 | Rule-out (0/2 h, Reichlin 2015) |
| 1929 | 5 | 6 | Rule-out (hsTnT and Copeptin 0 h, Moeckel 2014) |
| 1930 | 6 | | Rule-out (ESC 0 h) |
| 1931 | 53 | 55 | Rule-in (ESC 0 h) |
| 1932 | 7 | 7 | Rule-out (ESC 0/1 h) |
| 1933 | 6 | 6 | Rule-out (ESC 0/1 h) |
| 1934 | 117 | 205 | Rule-in (ESC 0 h) |
| 1935 | 172 | 163 | Rule-in (ESC 0 h) |
| 1936 | 71 | 80 | Rule-in (ESC 0 h) |
| 1937 | 27 | 29 | Observation zone (ESC 0/1 h) |
| 1938 | 16 | 1006 | Rule-in (ESC 0/3 h) |
| 1939 | 20 | 40 | Rule-in (ESC 0/1 h) |
| 1940 | 7 | 6 | Rule-out (ESC 0/1 h) |
| 1941 | 8 | 6 | Rule-out (ESC 0/1 h) |
| 1942 | 7 | 9 | Rule-out (ESC 0/1 h) |
| 1943 | 15 | 13 | Observation zone (ESC 0/1 h) |
| 1944 | 123 | 115 | Rule-in (ESC 0 h) |
| 1945 | 14 | 14 | Observation zone (0/2 h, Reichlin 2015) |
| 1946 | 13 | 11 | Observation zone (ESC 0/1 h) |
| 1947 | 5 | 1.5 | Observation zone (ESC 0/1 h) |
| 1948 | 13 | 14 | Observation zone (ESC 0/1 h) |
| 1949 | 8 | 11 | Observation zone (ESC 0/1 h) |
| 1950 | 6 | 4 | Rule-out (ESC 0/1 h) |
| 1951 | 7 | 8 | Rule-out (ESC 0/1 h) |
| 1952 | 1.5 | 10 | Observation zone (0/2 h, Reichlin 2015) |
| 1953 | 5 | 4 | Rule-out (0/2 h, Reichlin 2015) |
| 1954 | 11 | 13 | Rule-out (ESC 0/1 h) |
| 1955 | 7 | 7 | Rule-out (ESC 0/1 h) |
| 1956 | 5 | 4 | Rule-out (ESC 0/1 h) |
| 1957 | 165 | 137 | Rule-in (ESC 0 h) |
| 1958 | 10 | 9 | Rule-out (hsTnT and Copeptin 0 h, Moeckel 2014) |
| 1959 | 6 | 6 | Rule-out (hsTnT and Copeptin 0 h, Moeckel 2014) |
| 1960 | 17 | 15 | Observation zone (ESC 0/1 h) |
| 1961 | 13 | 11 | Observation zonc (ESC 0/1 h) |
| 1962 | 5 | 7 | Rule-out (ESC 0/1 h) |
| 1963 | 5 | 7 | Rule-out (ESC 0/1 h) |
| 1964 | 1.5 | 6 | Observation zone (ESC 0/1 h) |
| 1965 | 6 | 9 | Observation zone (ESC 0/1 h) |
| 1966 | 55 | 47 | Rule-in (ESC 0 h) |
| 1967 | 7 | 8 | Rule-out (0/2 h, Reichlin 2015) |
| 1968 | 17 | 12 | Rule-in (ESC 0/1 h) |
| 1969 | 5 | 3 | Rule-out (ESC 0/1 h) |
| 1970 | 25 | 26 | Rule-out (ESC 0/3 h) |
| 1971 | 9 | 11 | Rule-out (ESC 0/1 h) |
| 1972 | 6 | 5 | Rule-out (ESC 0/1 h) |
| 1973 | 10 | 10 | Rule-out (0/2 h, Reichlin 2015) |
| 1974 | 594 | 522 | Rule-in (ESC 0 h) |
| 1975 | 6 | 8 | Rule-out (0/2 h, Reichlin 2015) |
| 1976 | 19 | 18 | Observation zone (ESC 0/1 h) |
| 1977 | 5 | 5 | Rule-out (ESC 0/1 h) |
| 1978 | 7 | 6 | Rule-out (ESC 0/1 h) |
| 1979 | 27 | 35 | Rule-in (ESC 0/1 h) |
| 1980 | 6 | 3 | Observation zone (ESC 0/1 h) |
| 1981 | 8 | 6 | Rule-out (hsTnT and Copeptin 0 h, Moeckel 2014) |
| 1982 | 10 | 9 | Rule-out (hsTnT and Copeptin 0 h, Moeckel 2014) |
| 1983 | 5 | 1.5 | Observation zone (ESC 0/1 h) |
| 1984 | 11 | 12 | Rule-out (ESC 0/1 h) |
| 1985 | 9 | 6 | Rule-out (ESC 0/3 h) |
| 1986 | 6 | 1.5 | Observation zone (ESC 0/1 h) |
| 1987 | 9 | 8 | Rule-out (0/2 h, Reichlin 2015) |
| 1988 | 13 | 12 | Rule-out (0/2 h, Reichlin 2015) |
| 1989 | 163 | | Rule-in (ESC 0 h) |
| 1990 | 16 | 15 | Observation zone (ESC 0/1 h) |
| 1991 | 7 | 7 | Rule-out (hsTnT and Copeptin 0 h, Moeckel 2014) |
| 1992 | 230 | 635 | Rule-in (ESC 0 h) |
| 1993 | 250 | 551 | Rule-in (ESC 0 h) |
| 1994 | 25 | 32 | Rule-in (ESC 0/1 h) |
| 1995 | 9 | 9 | Rule-out (ESC 0/1 h) |
| 1996 | 11 | 13 | Rule-out (ESC 0/1 h) |
| 1997 | 33 | 34 | Observation zone (ESC 0/1 h) |
| 1998 | 89 | 117 | Rule-in (ESC 0 h) |
| 1999 | 11 | 10 | Rule-out (ESC 0/1 h) |
| 2000 | 7 | 1.5 | Rule-in (ESC 0/1 h) |
| 2001 | 37 | 50 | Rule-in (ESC 0/1 h) |
| 2002 | 5 | 5 | Rule-out (ESC 0/1 h) |
| 2003 | 4 | 7 | Rule-out (0/2 h, Reichlin 2015) |
| 2004 | 7 | 4 | Observation zone (ESC 0/1 h) |
| 2005 | 13 | 16 | Rule-out (ESC 0/3 h) |
| 2006 | 13 | 14 | Observation zone (0/2 h, Reichlin 2015) |
| 2007 | 5 | 6 | Rule-out (0/2 h, Reichlin 2015) |
| 2008 | 5 | 6 | Rule-out (ESC 0/1 h) |
| 2009 | 17 | 14 | Observation zone (0/2 h, Reichlin 2015) |
| 2010 | 28 | 251 | Rule-in (ESC 0/3 h) |
| 2011 | 10 | 32 | Rule-in (ESC 0/3 h) |
| 2012 | 6 | 6 | Rule-out (ESC 0/1 h) |
| 2013 | 20 | 22 | Observation zone (ESC 0/1 h) |
| 2014 | 18 | 15 | Observation zone (ESC 0/1 h) |
| 2015 | 5 | 7 | Rule-out (0/2 h, Reichlin 2015) |
| 2016 | 78 | 106 | Rule-in (ESC 0 h) |
| 2017 | 18 | 17 | Observation zonc (ESC 0/1 h) |
| 2018 | 6 | 7 | Rule-out (ESC 0/1 h) |
| 2019 | 14 | 13 | Observation zone (ESC 0/1 h) |
| 2020 | 65 | 66 | Rule-in (ESC 0 h) |
| 2021 | 6 | 6 | Rule-out (ESC 0/1 h) |
| 2022 | 7 | 7 | Rule-out (ESC 0 h) |
| 2023 | 30 | 31 | Observation zonc (ESC 0/1 h) |
| 2024 | 15 | 14 | Rule-out (ESC 0/3 h) |
| 2025 | 21 | 20 | Observation zone (ESC 0/1 h) |
| 2026 | 6 | 6 | Rule-out (0/2 h, Reichlin 2015) |
| 2027 | 22 | 9 | Rule-out (ESC 0/3 h) |
| 2028 | 7 | 10 | Rule-out (0/2 h, Reichlin 2015) |
| 2029 | 7 | 8 | Rule-out (ESC 0/1 h) |
| 2030 | 7 | 8 | Rule-out (ESC 0/1 h) |
| 2031 | 7 | 1.5 | Rule-in (ESC 0/1 h) |
| 2032 | 103 | 102 | Rule-in (ESC 0 h) |
| 2033 | 23 | 20 | Observation zone (ESC 0/1 h) |
| 2034 | 7 | 7 | Rule-out (ESC 0/1 h) |
| 2035 | 12 | | Rule-out (ESC 0 h) |
| 2036 | 10 | 5 | Rule-in (ESC 0/1 h) |
| 2037 | 10 | 10 | Rule-out (ESC 0/1 h) |
| 2038 | 8 | 13 | Rule-out (ESC 0 h) |
| 2039 | 5 | | Rule-out (ESC 0 h) |
| 2040 | 19 | 18 | Observation zone (0/2 h, Reichlin 2015) |
| 2041 | 39 | 47 | Rule-in (ESC 0/3 h) |
| 2042 | 14 | 11 | Observation zone (ESC 0/1 h) |
| 2043 | 1.5 | | Rule-out (hsTnT and Copeptin 0 h, Moeckel 2014) |
| 2044 | 3 | 8 | Rule-in (ESC 0/1 h) |
| 2045 | 13 | 10 | Rule-out (ESC 0 h) |
| 2046 | 1.5 | 1.5 | Rule-out (ESC 0/1 h) |
| 2047 | 29 | 29 | Observation zone (0/2 h, Reichlin 2015) |
| 2048 | 6 | 5 | Rule-out (ESC 0 h) |
| 2049 | 6 | 7 | Rule-out (ESC 0 h) |
| 2050 | 60 | 67 | Rule-in (ESC 0 h) |
| 2051 | 14 | 9 | Rule-in (ESC 0/1 h) |
| 2052 | 6 | 6 | Rule-out (ESC 0 h) |
| 2053 | 15 | 129 | Rule-in (ESC 0/1 h) |
| 2054 | 6 | 6 | Rule-out (ESC 0/1 h) |
| 2055 | 10 | 10 | Rule-out (0/2 h, Reichlin 2015) |
| 2056 | 28 | 119 | Rule-in (ESC 0/1 h) |
| 2057 | 7 | 8 | Rule-out (0/2 h, Reichlin 2015) |
| 2058 | 10 | 9 | Rule-out (ESC 0 h) |
| 2059 | 999 | 977 | Rule-in (ESC 0 h) |
| 2060 | 1.5 | 8 | Rule-in (ESC 0/1 h) |
| 2061 | 5 | 1.5 | Observation zone (ESC 0/1 h) |
| 2062 | 11 | 7 | Observation zone (ESC 0/1 h) |
| 2063 | 1.5 | | Rule-out (ESC 0 h) |
| 2064 | 21 | 25 | Rule-out (ESC 0/3 h) |
| 2065 | 10 | 12 | Rule-out (0/2 h, Reichlin 2015) |
| 2066 | 6 | 7 | Rule-out (ESC 0 h) |
| 2067 | 16 | 15 | Observation zone (ESC 0/1 h) |
| 2068 | 15 | 16 | Observation zone (ESC 0/1 h) |
| 2069 | 25 | 24 | Observation zone (ESC 0/1 h) |
| 2070 | 8 | 9 | Rule-out (ESC 0/1 h) |
| 2071 | 10 | 7 | Observation zone (ESC 0/1 h) |
| 2072 | 10 | 5 | Observation zone (0/2 h, Reichlin 2015) |
| 2073 | 4 | 6 | Rule-out (hsTnT and Copeptin 0 h, Mocckel 2014) |
| 2074 | 8 | 7 | Rule-out (ESC 0 h) |
| 2075 | 6 | 7 | Rule-out (hsTnT and Copeptin 0 h, Moeckel 2014) |
| 2076 | 21 | 22 | Rule-out (ESC 0/3 h) |
| 2077 | 269 | 270 | Rule-in (ESC 0 h) |
| 2078 | 6 | 7 | Rule-out (ESC 0 h) |
| 2079 | 9 | 10 | Rule-out (ESC 0/1 h) |
| 2080 | 8 | | Rule-out (ESC 0 h) |
| 2081 | 11 | 11 | Rule-out (ESC 0/1 h) |
| 2082 | 6 | 8 | Rule-out (ESC 0/1 h) |
| 2083 | 1.5 | 3 | Rule-out (ESC 0 h) |
| 2084 | 8 | 5 | Observation zone (ESC 0/1 h) |
| 2085 | 6 | | Rule-out (ESC 0 h) |
| 2086 | 4 | | Rule-out (ESC 0 h) |
| 2087 | 14 | 23 | Rule-in (ESC 0/3 h) |
| 2088 | 187 | 117 | Rule-in (ESC 0 h) |
| 2089 | 7 | 1.5 | Rule-out (ESC 0 h) |
| 2090 | 10 | 8 | Rule-out (hsTnT and Copeptin 0 h, Moeckel 2014) |
| 2091 | 5 | 4 | Rule-out (hsTnT and Copcptin 0 h, Mocckel 2014) |
| 2092 | 5 | 4 | Rule-out (0/2 h, Reichlin 2015) |
| 2093 | 8 | 7 | Rule-out (ESC 0/1 h) |
| 2094 | 3 | 8 | Rule-out (ESC 0 h) |
| 2095 | 8 | 6 | Rule-out (ESC 0/1 h) |
| 2096 | 6 | 4 | Rule-out (ESC 0/1 h) |
| 2097 | 9 | | Rule-out (ESC 0 h) |
| 2098 | 10 | 11 | Rule-out (ESC 0 h) |
| 2099 | 5 | 7 | Rule-out (hsTnT and Copeptin 0 h, Moeckel 2014) |
| 2100 | 9 | 6 | Rule-out (ESC 0 h) |
| 2101 | 5 | | Rule-out (ESC 0 h) |
| 2102 | 9 | 9 | Rule-out (ESC 0 h) |
| 2103 | 21 | 26 | Rule-in (ESC 0/1 h) |
| 2104 | 6 | 6 | Rule-out (ESC 0/3 h) |
| 2105 | 8 | 7 | Rule-out (ESC 0 h) |
| 2106 | 4 | 1.5 | Rule-out (ESC 0/1 h) |
| 2107 | 45 | 45 | Observation zone (ESC 0/1 h) |
| 2108 | 7 | 7 | Rule-out (ESC 0 h) |
| 2109 | 12 | 14 | Observation zone (ESC 0/1 h) |
| 2110 | 79 | 110 | Rule-in (ESC 0 h) |
| 2111 | 9 | 8 | Rule-out (ESC 0 h) |
| 2112 | 6 | 4 | Rule-out (ESC 0/1 h) |
| 2113 | 10 | 7 | Observation zone (ESC 0/1 h) |
| 2114 | 71 | 71 | Rule-in (ESC 0 h) |
| 2115 | 1.5 | | Rule-out (hsTnT and Copeptin 0 h, Moeckel 2014) |
| 2116 | 24 | 26 | Observation zone (0/2 h, Reichlin 2015) |
| 2117 | 95 | 120 | Rule-in (ESC 0 h) |
| 2118 | 12 | 11 | Rule-out (ESC 0 h) |
| 2119 | 10 | 7 | Rule-out (ESC 0 h) |
| 2120 | 6 | 4 | Rule-out (ESC 0/1 h) |
| 2121 | 9 | 4 | Rule-in (ESC 0/1 h) |
| 2122 | 13 | 10 | Rule-out (0/2 h, Reichlin 2015) |
| 2123 | 4 | | Rule-out (ESC 0 h) |
| 2124 | 6 | 5 | Rule-out (ESC 0 h) |
| 2125 | 152 | 263 | Rule-in (ESC 0 h) |
| 2126 | 7 | 7 | Rule-out (ESC 0/1 h) |
| 2127 | 24 | 23 | Observation zone (ESC 0/1 h) |
| 2128 | 125 | | Rule-in (ESC 0 h) |
| 2129 | 13 | 12 | Rule-out (hsTnT and Copeptin 0 h, Mocckel 2014) |
| 2130 | 15 | 24 | Rule-in (ESC 0/1 h) |
| 2131 | 85 | 219 | Rule-in (ESC 0 h) |
| 2132 | 21 | 19 | Rule-out (ESC 0/3 h) |
| 2133 | 1.5 | 4 | Rule-out (ESC 0/1 h) |
| 2134 | 7 | | Rule-out (ESC 0 h) |
| 2135 | 22 | 18 | Observation zonc (ESC 0/1 h) |
| 2136 | 1.5 | 1.5 | Rule-out (0/2 h, Reichlin 2015) |
| 2137 | 1.5 | 4 | Rule-out (ESC 0/1 h) |
| 2138 | 178 | 175 | Rule-in (ESC 0 h) |
| 2139 | 82 | 149 | Rule-in (ESC 0 h) |
| 2140 | 26 | 20 | Rule-in (ESC 0/1 h) |
| 2141 | 144 | 234 | Rule-in (ESC 0 h) |
| 2142 | 1.5 | 4 | Rule-out (0/2 h, Reichlin 2015) |
| 2143 | 42 | 102 | Rule-in (ESC 0/1 h) |
| 2144 | 9 | 6 | Observation zone (ESC 0/1 h) |
| 2145 | 9 | 7 | Rule-out (ESC 0/1 h) |
| 2146 | 587 | | Rule-in (ESC 0 h) |
| 2147 | 25 | 26 | Observation zonc (ESC 0/1 h) |
| 2148 | 39 | 130 | Rule-in (0/2 h, Reichlin 2015) |
| 2149 | 14 | 12 | Observation zone (0/2 h, Reichlin 2015) |
| 2150 | 4 | | Rule-out (ESC 0 h) |
| 2151 | 7 | 4 | Observation zone (ESC 0/1 h) |
| 2152 | 6 | 7 | Rule-out (ESC 0 h) |
| 2153 | 13 | 4 | Rule-in (ESC 0/1 h) |
| 2154 | 5 | 7 | Rule-out (hsTnT and Copeptin 0 h, Moeckel 2014) |
| 2155 | 43 | 42 | Observation zone (ESC 0/1 h) |
| 2156 | 1.5 | 3 | Rule-out (ESC 0/1 h) |
| 2157 | 1.5 | | Rule-out (ESC 0 h) |
| 2158 | 29 | 34 | Observation zone (0/2 h, Reichlin 2015) |
| 2159 | 51 | 63 | Rule-in (ESC 0/1 h) |
| 2160 | 62 | | Rule-in (ESC 0 h) |
| 2161 | 30 | 121 | Rule-in (ESC 0/1 h) |
| 2162 | 39 | 32 | Observation zone (0/2 h, Reichlin 2015) |
| 2163 | 1.5 | | Rule-out (ESC 0 h) |
| 2164 | 9 | 9 | Rule-out (ESC 0 h) |
| 2165 | 4 | 1.5 | Rule-out (ESC 0 h) |
| 2166 | 24 | 20 | Observation zone (ESC 0/1 h) |
| 2167 | 6 | 7 | Rule-out (ESC 0/1 h) |
| 2168 | 1.5 | 5 | Rule-out (0/2 h, Reichlin 2015) |
| 2169 | 5 | 6 | Rule-out (ESC 0/1 h) |
| 2170 | 6 | 8 | Rule-out (ESC 0/1 h) |
| 2171 | 1.5 | 1.5 | Rule-out (ESC 0 h) |
| 2172 | 4 | 1.5 | Rule-out (ESC 0/1 h) |
| 2173 | 13 | 15 | Observation zone (ESC 0/1 h) |
| 2174 | 1.5 | 6 | Observation zone (ESC 0/1 h) |
| 2175 | 11 | 13 | Rule-out (ESC 0/1 h) |
| 2176 | 1.5 | 1.5 | Rule-out (ESC 0 h) |
| 2177 | 1.5 | | Rule-out (ESC 0 h) |
| 2178 | 5 | | Rule-out (ESC 0 h) |
| 2179 | 1.5 | | Rule-out (ESC 0 h) |
| 2180 | 14 | | Rule-out (ESC 0 h) |
| 2181 | 8 | 12 | Observation zone (ESC 0/1 h) |
| 2182 | 6 | 10 | Observation zone (ESC 0/1 h) |
| 2183 | 6 | 1.5 | Observation zone (ESC 0/1 h) |
| 2184 | 127 | 113 | Rule-in (ESC 0 h) |
| 2185 | 1.5 | | Rule-out (hsTnT and Copcptin 0 h, Mocckel 2014) |
| 2186 | 5 | 8 | Rule-out (ESC 0 h) |
| 2187 | 25 | 26 | Observation zone (ESC 0/1 h) |
| 2188 | 1.5 | | Rule-out (ESC 0 h) |
| 2189 | 81 | 113 | Rule-in (ESC 0 h) |
| 2190 | 7 | 12 | Observation zone (0/2 h, Reichlin 2015) |
| 2191 | 116 | 98 | Rule-in (ESC 0 h) |
| 2192 | 3 | 7 | Observation zone (0/2 h, Reichlin 2015) |
| 2193 | 12 | 12 | Rule-out (ESC 0/3 h) |
| 2194 | 60 | 73 | Rule-in (ESC 0 h) |
| 2195 | 86 | 631 | Rule-in (ESC 0 h) |
| 2196 | 1.5 | | Rule-out (ESC 0 h) |
| 2197 | 7 | 14 | Rule-in (ESC 0/1 h) |
| 2198 | 9 | 17 | Rule-in (ESC 0/1 h) |
| 2199 | 5 | 5 | Rule-out (ESC 0/1 h) |
| 2200 | 1.5 | 1.5 | Rule-out (ESC 0/1 h) |
| 2201 | 14 | 18 | Rule-out (ESC 0/3 h) |
| 2202 | 1.5 | | Rule-out (ESC 0 h) |
| 2203 | 7 | 4 | Observation zone (ESC 0/1 h) |
| 2204 | 7 | 7 | Rule-out (ESC 0/1 h) |
| 2205 | 4 | 6 | Rule-out (ESC 0 h) |
| 2206 | 1.5 | | Rule-out (ESC 0 h) |
| 2207 | 1.5 | | Rule-out (ESC 0 h) |
| 2208 | 76 | | Rule-in (ESC 0 h) |
| 2209 | 1.5 | | Rule-out (ESC 0 h) |
| 2210 | 90 | 78 | Rule-in (ESC 0 h) |
| 2211 | 1.5 | | Rule-out (ESC 0 h) |
| 2212 | 12 | 12 | Observation zone (ESC 0/1 h) |
| 2213 | 6 | 3 | Rule-out (ESC 0/3 h) |
| 2214 | 12 | 8 | Observation zone (0/2 h, Reichlin 2015) |
| 2215 | 1.5 | | Rule-out (ESC 0 h) |
| 2216 | 22 | 18 | Rule-out (ESC 0/3 h) |
| 2217 | 1.5 | 1.5 | Rule-out (ESC 0/1 h) |
| 2218 | 4 | 6 | Rule-out (hsTnT and Copeptin 0 h, Moeckel 2014) |
| 2219 | 13 | 12 | Observation zonc (ESC 0/1 h) |
| 2220 | 18 | 24 | Rule-in (ESC 0/1 h) |
| 2221 | 12 | 13 | Rule-out (ESC 0/3 h) |
| 2222 | 7 | 1.5 | Rule-in (ESC 0/1 h) |
| 2223 | 12 | 15 | Rule-out (ESC 0/3 h) |
| 2224 | 46 | 43 | Observation zone (0/2 h, Reichlin 2015) |
| 2225 | 27 | 27 | Observation zone (ESC 0/1 h) |
| 2226 | 15 | 14 | Rule-out (ESC 0/3 h) |
| 2227 | 72 | | Rule-in (ESC 0 h) |
| 2228 | 4 | 7 | Rule-out (0/2 h, Reichlin 2015) |
| 2229 | 29 | 37 | Rule-in (ESC 0/3 h) |
| 2230 | 3 | | Rule-out (hsTnT and Copeptin 0 h, Moeckel 2014) |
| 2231 | 8 | 17 | Rule-in (ESC 0/3 h) |
| 2232 | 5 | 7 | Rule-out (ESC 0/1 h) |
| 2233 | 11 | 11 | Rule-out (ESC 0/1 h) |
| 2234 | 6 | 6 | Rule-out (ESC 0/1 h) |
| 2235 | 5 | 6 | Rule-out (0/2 h, Reichlin 2015) |
| 2236 | 7 | 8 | Rule-out (ESC 0/1 h) |
| 2237 | 1.5 | | Rule-out (ESC 0 h) |
| 2238 | 4 | 3 | Rule-out (hsTnT and Copeptin 0 h, Moeckel 2014) |
| 2239 | 4 | 1.5 | Rule-out (ESC 0 h) |
| 2240 | 4 | 1.5 | Rule-out (ESC 0/1 h) |
| 2241 | 1.5 | | Rule-out (ESC 0 h) |
| 2242 | 227 | 260 | Rule-in (ESC 0 h) |
| 2243 | 3 | | Rule-out (ESC 0 h) |
| 2244 | 35 | 36 | Rule-in (ESC 0/3 h) |
| 2245 | 300 | | Rule-in (ESC 0 h) |
| 2246 | 91 | 871 | Rule-in (ESC 0 h) |
| 2247 | 5 | 3 | Rule-out (ESC 0/1 h) |
| 2248 | 6 | | Rule-out (ESC 0 h) |
| 2249 | 18 | 17 | Rule-out (ESC 0/3 h) |
| 2250 | 4 | 4 | Rule-out (ESC 0 h) |
| 2251 | 8 | 8 | Rule-out (ESC 0 h) |
| 2252 | 339 | | Rule-in (ESC 0 h) |
| 2253 | 4 | 4 | Rule-out (ESC 0/1 h) |
| 2254 | 13 | | Rule-out (ESC 0 h) |
| 2255 | 13 | | Rule-out (ESC 0 h) |
| 2256 | 4 | 4 | Rule-out (ESC 0 h) |
| 2257 | 1.5 | 1.5 | Rule-out (ESC 0 h) |
| 2258 | 5 | 4 | Rule-out (ESC 0 h) |
| 2259 | 10 | 10 | Rule-out (ESC 0/1 h) |
| 2260 | 4 | | Rule-out (ESC 0 h) |
| 2261 | 51 | 51 | Rule-out (ESC 0/3 h) |
| 2262 | 158 | 191 | Rule-in (ESC 0 h) |
| 2263 | 10 | 11 | Rule-out (ESC 0 h) |
| 2264 | 6 | 5 | Rule-out (hsTnT and Copeptin 0 h, Moeckel 2014) |
| 2265 | 10 | | Rule-out (ESC 0 h) |
| 2266 | 33 | 36 | Observation zone (ESC 0/1 h) |
| 2267 | 9 | 10 | Rule-out (ESC 0/1 h) |
| 2268 | 54 | 57 | Rule-in (ESC 0 h) |
| 2269 | 3 | 1.5 | Rule-out (ESC 0/1 h) |
| 2270 | 3 | | Rule-out (ESC 0 h) |
| 2271 | 4 | 1.5 | Rule-out (ESC 0 h) |
| 2272 | 5 | 5 | Rule-out (0/2 h, Reichlin 2015) |
| 2273 | 1.5 | 1.5 | Rule-out (ESC 0/1 h) |
| 2274 | 10 | 11 | Rule-out (0/2 h, Reichlin 2015) |
| 2275 | 36 | 33 | Observation zonc (ESC 0/1 h) |
| 2276 | 37 | 39 | Observation zone (ESC 0/1 h) |
| 2277 | 10 | 12 | Rule-out (ESC 0/1 h) |
| 2278 | 18 | 15 | Observation zone (ESC 0/1 h) |
| 2279 | 1.5 | | Rule-out (ESC 0 h) |
| 2280 | 4 | 4 | Rule-out (ESC 0/1 h) |
| 2281 | 6 | 5 | Rule-out (ESC 0 h) |
| 2282 | 5 | 6 | Rule-out (ESC 0/1 h) |
| 2283 | 27 | 25 | Observation zone (0/2 h, Reichlin 2015) |
| 2284 | 21 | 22 | Observation zone (ESC 0/1 h) |
| 2285 | 5 | | Rule-out (ESC 0 h) |
| 2286 | 14 | 22 | Rule-in (ESC 0/1 h) |
| 2287 | 645 | 816 | Rule-in (ESC 0 h) |
| 2288 | 5 | 4 | Rule-out (0/2 h, Reichlin 2015) |
| 2289 | 1.5 | | Rule-out (ESC 0 h) |
| 2290 | 3 | | Rule-out (ESC 0 h) |
| 2291 | 157 | 144 | Rule-in (ESC 0 h) |
| 2292 | 7 | 6 | Rule-out (0/2 h, Reichlin 2015) |
| 2293 | 4 | 6 | Rule-out (0/2 h, Reichlin 2015) |
| 2294 | 16 | 14 | Observation zone (ESC 0/1 h) |
| 2295 | 7 | 6 | Rule-out (0/2 h, Reichlin 2015) |
| 2296 | 6 | 5 | Rule-out (ESC 0 h) |
| 2297 | 11 | 9 | Rule-out (ESC 0/1 h) |
| 2298 | 963 | 1141 | Rule-in (ESC 0 h) |
| 2299 | 5 | | Rule-out (ESC 0 h) |
| 2300 | 15 | 11 | Observation zone (0/2 h, Reichlin 2015) |
| 2301 | 1.5 | 4 | Rule-out (0/2 h, Reichlin 2015) |
| 2302 | 7 | 7 | Rule-out (hsTnT and Copeptin 0 h, Moeckel 2014) |
| 2303 | 1.5 | 3 | Rule-out (0/2 h, Reichlin 2015) |
| 2304 | 8 | 9 | Rule-out (ESC 0 h) |
| 2305 | 4 | 1.5 | Rule-out (ESC 0 h) |
| 2306 | 9 | 11 | Rule-out (ESC 0 h) |
| 2307 | 5 | 4 | Rule-out (ESC 0/1 h) |
| 2308 | 7 | 7 | Rule-out (0/2 h, Reichlin 2015) |
| 2309 | 10 | 10 | Rule-out (ESC 0/1 h) |
| 2310 | 1.5 | 4 | Rule-out (ESC 0 h) |
| 2311 | 4 | | Rule-out (ESC 0 h) |
| 2312 | 11 | 10 | Rule-out (0/2 h, Reichlin 2015) |
| 2313 | 9 | 8 | Rule-out (ESC 0 h) |
| 2314 | 3 | 1.5 | Rule-out (ESC 0/1 h) |
| 2315 | 127 | 151 | Rule-in (ESC 0 h) |
| 2316 | 12 | 13 | Observation zone (ESC 0/1 h) |
| 2317 | 10 | 8 | Rule-out (ESC 0 h) |
| 2318 | 17 | 19 | Observation zone (ESC 0/1 h) |
| 2319 | 20 | 21 | Observation zone (0/2 h, Reichlin 2015) |
| 2320 | 9 | 11 | Rule-out (0/2 h, Reichlin 2015) |
| 2321 | 4 | 5 | Rule-out (ESC 0 h) |
| 2322 | 8 | 7 | Rule-out (hsTnT and Copeptin 0 h, Moeckel 2014) |
| 2323 | 20 | 27 | Observation zone (0/2 h, Reichlin 2015) |
| 2324 | 4 | 3 | Rule-out (0/2 h, Reichlin 2015) |
| 2325 | 11 | 14 | Observation zonc (ESC 0/1 h) |
| 2326 | 41 | 40 | Observation zone (ESC 0/1 h) |
| 2327 | 13 | 15 | Observation zone (ESC 0/1 h) |
| 2328 | 43 | 42 | Observation zone (0/2 h, Reichlin 2015) |
| 2329 | 8 | 7 | Rule-out (ESC 0/1 h) |
| 2330 | 5 | | Rule-out (ESC 0 h) |
| 2331 | 5 | 4 | Rule-out (ESC 0 h) |
| 2332 | 10 | 12 | Rule-out (ESC 0/1 h) |
| 2333 | 24 | 25 | Observation zone (ESC 0/1 h) |
| 2334 | 30 | 33 | Observation zone (0/2 h, Reichlin 2015) |
| 2335 | 4 | 1.5 | Rule-out (ESC 0/1 h) |
| 2336 | 5 | 1.5 | Observation zone (ESC 0/1 h) |
| 2337 | 16 | 14 | Observation zone (ESC 0/1 h) |
| 2338 | 5 | 4 | Rule-out (ESC 0/3 h) |
| 2339 | 5 | 4 | Rule-out (ESC 0/1 h) |
| 2340 | 4 | 5 | Rule-out (ESC 0 h) |
| 2341 | 7 | 7 | Rule-out (ESC 0 h) |
| 2342 | 808 | 2042 | Rule-in (ESC 0 h) |
| 2343 | 9 | 10 | Rule-out (ESC 0/1 h) |
| 2344 | 33 | 139 | Rule-in (ESC 0/1 h) |
| 2345 | 6 | 7 | Rule-out (hsTnT and Copeptin 0 h, Moeckel 2014) |
| 2346 | 3 | | Rule-out (ESC 0 h) |
| 2347 | 21 | 129 | Rule-in (0/2 h, Reichlin 2015) |
| 2348 | 7 | 8 | Rule-out (ESC 0/1 h) |
| 2349 | 8 | 10 | Rule-out (ESC 0/1 h) |
| 2350 | 8 | 6 | Rule-out (ESC 0/1 h) |
| 2351 | 28 | 26 | Observation zone (ESC 0/1 h) |
| 2352 | 12 | 12 | Observation zone (ESC 0/1 h) |
| 2353 | 8 | 9 | Rule-out (ESC 0/3 h) |
| 2354 | 31 | 31 | Rule-out (ESC 0/3 h) |
| 2355 | 5 | 5 | Rule-out (ESC 0/1 h) |
| 2356 | 14 | 16 | Rule-out (ESC 0/3 h) |
| 2357 | 5 | 7 | Rule-out (ESC 0/1 h) |
| 2358 | 153 | 1885 | Rule-in (ESC 0 h) |
| 2359 | 14 | 13 | Observation zone (0/2 h, Reichlin 2015) |
| 2360 | 8 | 9 | Rule-out (ESC 0 h) |
| 2361 | 5 | | Rule-out (ESC 0 h) |
| 2362 | 14 | 36 | Rule-in (ESC 0/3 h) |
| 2363 | 25 | 35 | Rule-in (ESC 0/1 h) |
| 2364 | 6 | 1.5 | Observation zone (0/2 h, Reichlin 2015) |
| 2365 | 5 | 7 | Rule-out (ESC 0/1 h) |
| 2366 | 7 | 5 | Rule-out (0/2 h, Reichlin 2015) |
| 2367 | 5 | 6 | Rule-out (ESC 0 h) |
| 2368 | 7 | 6 | Rule-out (ESC 0 h) |
| 2369 | 280 | 239 | Rule-in (ESC 0 h) |
| 2370 | 4085 | 6538 | Rule-in (ESC 0 h) |
| 2371 | 1.5 | 3 | Rule-out (ESC 0 h) |
| 2372 | 7 | 6 | Rule-out (ESC 0 h) |
| 2373 | 10 | 9 | Rule-out (ESC 0/1 h) |
| 2374 | 4 | 4 | Rule-out (ESC 0 h) |
| 2375 | 10 | 8 | Rule-out (ESC 0/1 h) |
| 2376 | 1.5 | 1.5 | Rule-out (ESC 0/1 h) |
| 2377 | 1.5 | | Rule-out (ESC 0 h) |
| 2378 | 4 | | Rule-out (ESC 0 h) |
| 2379 | 5 | 6 | Rule-out (ESC 0/1 h) |
| 2380 | 10 | 12 | Rule-out (0/2 h, Reichlin 2015) |
| 2381 | 13 | 29 | Rule-in (ESC 0/3 h) |
| 2382 | 20 | 20 | Observation zone (ESC 0/1 h) |
| 2383 | 11 | 10 | Rule-out (0/2 h, Reichlin 2015) |
| 2384 | 1.5 | 5 | Rule-out (ESC 0 h) |
| 2385 | 11 | 11 | Rule-out (ESC 0 h) |
| 2386 | 1.5 | 1.5 | Rule-out (ESC 0 h) |
| 2387 | 5 | 4 | Rule-out (ESC 0 h) |
| 2388 | 7 | 9 | Rule-out (ESC 0 h) |
| 2389 | 4 | 4 | Rule-out (ESC 0 h) |
| 2390 | 13 | 30 | Rule-in (ESC 0/1 h) |
| 2391 | 4 | 3 | Rule-out (ESC 0/1 h) |
| 2392 | 6 | 6 | Rule-out (ESC 0 h) |
| 2393 | 4 | | Rule-out (ESC 0 h) |
| 2394 | 5 | 5 | Rule-out (ESC 0/1 h) |
| 2395 | 4 | | Rule-out (ESC 0 h) |
| 2396 | 20 | 47 | Rule-in (ESC 0/1 h) |
| 2397 | 19 | 18 | Observation zone (ESC 0/1 h) |
| 2398 | 3 | | Rule-out (ESC 0 h) |
| 2399 | 12 | 10 | Observation zonc (ESC 0/1 h) |
| 2400 | 8 | 7 | Rule-out (ESC 0 h) |
| 2401 | 5 | | Rule-out (ESC 0 h) |
| 2402 | 1.5 | 4 | Rule-out (ESC 0/1 h) |
| 2403 | 1.5 | 1.5 | Rule-out (hsTnT and Copeptin 0 h, Moeckel 2014) |
| 2404 | 5 | 6 | Rule-out (ESC 0 h) |
| 2405 | 12 | 14 | Observation zone (0/2 h, Reichlin 2015) |
| 2406 | 1.5 | 3 | Rule-out (ESC 0 h) |
| 2407 | 6 | | Rule-out (ESC 0 h) |
| 2408 | 14 | 12 | Rule-out (ESC 0/3 h) |
| 2409 | 3 | 1.5 | Rule-out (hsTnT and Copcptin 0 h, Mocckel 2014) |
| 2410 | 5 | | Rule-out (ESC 0 h) |
| 2411 | 5 | 4 | Rule-out (0/2 h, Reichlin 2015) |
| 2412 | 9 | 11 | Rule-out (ESC 0 h) |
| 2413 | 5 | 4 | Rule-out (ESC 0/1 h) |
| 2414 | 9 | 16 | Rule-in (ESC 0/1 h) |
| 2415 | 28 | 49 | Rule-in (0/2 h, Reichlin 2015) |
| 2416 | 11 | 11 | Rule-out (hsTnT and Copeptin 0 h, Moeckel 2014) |
| 2417 | 1.5 | 4 | Rule-out (hsTnT and Copeptin 0 h, Moeckel 2014) |
| 2418 | 5 | 1.5 | Rule-out (ESC 0 h) |
| 2419 | 12 | 10 | Rule-out (ESC 0 h) |
| 2420 | 1.5 | | Rule-out (ESC 0 h) |
| 2421 | 25 | 22 | Rule-out (ESC 0/3 h) |
| 2422 | 12 | 10 | Rule-out (0/2 h, Reichlin 2015) |
| 2423 | 8 | | Rule-out (ESC 0 h) |
| 2424 | 43 | 39 | Rule-out (ESC 0/3 h) |
| 2425 | 9 | 5 | Observation zone (ESC 0/1 h) |
| 2426 | 94 | 77 | Rule-in (ESC 0 h) |
| 2427 | 184 | 242 | Rule-in (ESC 0 h) |
| 2428 | 9 | 10 | Rule-out (0/2 h, Reichlin 2015) |
| 2429 | 7 | 8 | Rule-out (ESC 0 h) |
| 2430 | 5 | 8 | Observation zone (ESC 0/1 h) |
| 2431 | 27 | 27 | Observation zone (ESC 0/1 h) |
| 2432 | 17 | 15 | Observation zone (ESC 0/1 h) |
| 2433 | 6 | 5 | Rule-out (ESC 0/1 h) |
| 2434 | 237 | | Rule-in (ESC 0 h) |
| 2435 | 6 | 5 | Rule-out (0/2 h, Reichlin 2015) |
| 2436 | 1.5 | 1.5 | Rule-out (0/2 h, Reichlin 2015) |
| 2437 | 13 | 12 | Rule-out (ESC 0 h) |
| 2438 | 9 | | Rule-out (ESC 0 h) |
| 2439 | 6 | 7 | Rule-out (ESC 0/1 h) |
| 2440 | 4 | | Rule-out (ESC 0 h) |
| 2441 | 1.5 | 3 | Rule-out (ESC 0/1 h) |
| 2442 | 3 | 7 | Observation zone (0/2 h, Reichlin 2015) |
| 2443 | 1.5 | | Rule-out (ESC 0 h) |
| 2444 | 3 | 4 | Rule-out (ESC 0 h) |
| 2445 | 5 | 4 | Rule-out (ESC 0/1 h) |
| 2446 | 1.5 | | Rule-out (ESC 0 h) |
| 2447 | 5 | 6 | Rule-out (ESC 0 h) |
| 2448 | 5 | 1.5 | Observation zone (ESC 0/1 h) |
| 2449 | 5 | 7 | Rule-out (0/2 h, Reichlin 2015) |
| 2450 | 9 | 9 | Rule-out (ESC 0 h) |
| 2451 | 7 | 8 | Rule-out (ESC 0 h) |
| 2452 | 5 | 7 | Rule-out (ESC 0/3 h) |
| 2453 | 1.5 | | Rule-out (ESC 0 h) |
| 2454 | 6 | 5 | Rule-out (ESC 0/1 h) |
| 2455 | 39 | 35 | Observation zonc (ESC 0/1 h) |
| 2456 | 8 | 6 | Rule-out (hsTnT and Copeptin 0 h, Moeckel 2014) |
| 2457 | 77 | 68 | Rule-in (ESC 0 h) |
| 2458 | 1.5 | 1.5 | Rule-out (0/2 h, Reichlin 2015) |
| 2459 | 9 | 8 | Rule-out (ESC 0 h) |
| 2460 | 3 | | Rule-out (ESC 0 h) |
| 2461 | 5 | 4 | Rule-out (ESC 0 h) |
| 2462 | 22 | 24 | Rule-out (ESC 0/3 h) |
| 2463 | 1.5 | 1.5 | Rule-out (ESC 0 h) |
| 2464 | 4 | 3 | Rule-out (ESC 0/1 h) |
| 2465 | 10 | 8 | Rule-out (0/2 h, Reichlin 2015) |
| 2466 | 5 | 1.5 | Rule-out (0/2 h, Reichlin 2015) |
| 2467 | 6 | 5 | Rule-out (ESC 0 h) |
| 2468 | 48 | 48 | Observation zone (0/2 h, Reichlin 2015) |
| 2469 | 1.5 | | Rule-out (ESC 0 h) |
| 2470 | 1.5 | 1.5 | Rule-out (0/2 h, Reichlin 2015) |
| 2471 | 1.5 | 5 | Rule-out (0/2 h, Reichlin 2015) |
| 2472 | 7 | 9 | Rule-out (0/2 h, Reichlin 2015) |
| 2473 | 5 | 5 | Rule-out (0/2 h, Reichlin 2015) |
| 2474 | 3 | 1.5 | Rule-out (ESC 0 h) |
| 2475 | 6 | 5 | Rule-out (hsTnT and Copeptin 0 h, Moeckel 2014) |
| 2476 | 4 | | Rule-out (ESC 0 h) |
| 2477 | 17 | 18 | Observation zone (0/2 h, Reichlin 2015) |
| 2478 | 5 | | Rule-out (ESC 0 h) |
| 2479 | 5 | 5 | Rule-out (ESC 0/1 h) |
| 2480 | 45 | 47 | Rule-out (ESC 0/3 h) |
| 2481 | 1.5 | 5 | Rule-out (ESC 0 h) |
| 2482 | 10 | 18 | Rule-in (ESC 0/3 h) |
| 2483 | 4 | | Rule-out (ESC 0 h) |
| 2484 | 1.5 | 3 | Rule-out (ESC 0/1 h) |
| 2485 | 4 | 5 | Rule-out (ESC 0/1 h) |
| 2486 | 14 | 13 | Observation zone (0/2 h, Reichlin 2015) |
| 2487 | 587 | 537 | Rule-in (ESC 0 h) |
| 2488 | 29 | 26 | Observation zone (0/2 h, Reichlin 2015) |
| 2489 | 1.5 | | Rule-out (ESC 0 h) |
| 2490 | 4 | 5 | Rule-out (ESC 0/1 h) |
| 2491 | 1.5 | | Rule-out (ESC 0 h) |
| 2492 | 7 | 5 | Rule-out (ESC 0 h) |
| 2493 | 28 | 32 | Observation zone (ESC 0/1 h) |
| 2494 | 17 | 16 | Observation zone (ESC 0/1 h) |
| 2495 | 5 | | Rule-out (ESC 0 h) |
| 2496 | 17 | 29 | Rule-in (ESC 0/1 h) |
| 2497 | 10 | 15 | Observation zone (0/2 h, Reichlin 2015) |
| 2498 | 4 | 3 | Rule-out (ESC 0/1 h) |
| 2499 | 67 | 59 | Rule-in (ESC 0 h) |
| 2500 | 7 | 4 | Observation zone (ESC 0/1 h) |
| 2501 | 3 | 1.5 | Rule-out (ESC 0 h) |
| 2502 | 5 | 6 | Rule-out (0/2 h, Reichlin 2015) |
| 2503 | 14 | 12 | Rule-out (ESC 0/3 h) |
| 2504 | 5 | | Rule-out (ESC 0 h) |
| 2505 | 4 | | Rule-out (ESC 0 h) |
| 2506 | 235 | 210 | Rule-in (ESC 0 h) |
| 2507 | 1.5 | | Rule-out (ESC 0 h) |
| 2508 | 1.5 | 1.5 | Rule-out (hsTnT and Copeptin 0 h, Moeckel 2014) |
| 2509 | 641 | 889 | Rule-in (ESC 0 h) |
| 2510 | 8 | | Rule-out (hsTnT and Copeptin 0 h, Moeckel 2014) |
| 2511 | 3 | | Rule-out (ESC 0 h) |
| 2512 | 22 | 33 | Rule-in (0/2 h, Reichlin 2015) |
| 2513 | 3 | | Rule-out (ESC 0 h) |
| 2514 | 6 | 9 | Observation zone (ESC 0/1 h) |
| 2515 | 3 | 3 | Rule-out (hsTnT and Copeptin 0 h, Moeckel 2014) |
| 2516 | 4 | | Rule-out (ESC 0 h) |
| 2517 | 1.5 | | Rule-out (ESC 0 h) |
| 2518 | 10 | 11 | Rule-out (ESC 0/1 h) |
| 2519 | 4 | | Rule-out (ESC 0 h) |
| 2520 | 3 | 5 | Rule-out (ESC 0 h) |
| 2521 | 1.5 | 4 | Rule-out (ESC 0 h) |
| 2522 | 5 | 5 | Rule-out (ESC 0/1 h) |
| 2523 | 5 | 4 | Rule-out (ESC 0/1 h) |
| 2524 | 5 | | Rule-out (ESC 0 h) |
| 2525 | 53 | 51 | Rule-in (ESC 0 h) |

### REFERENCES

1. Stoyanov K M, Biener M, Hund H, Mueller-Hennessen M, Vafaie M, Katus H A, Giannitsis E. Effects of crowding in the emergency department on the diagnosis and management of suspected acute coronary syndrome using rapid algorithms: an observational study. BMJ Open. 2020 Oct. 8; 10(10):e041757. doi: 10.1136/bmjopen-2020-041757. PMID: 33033102; PMCID: PMC7545662.
2. Collet J P, Thiele H, Barbato E, Barthélémy O, Bauersachs J, Bhatt D L, Dendale P, Dorobantu M, Edvardsen T, Folliguet T, Gale C P, Gilard M, Jobs A, Jini P, Lambrinou E, Lewis B S, Mehilli J, Meliga E, Merkely B, Mueller C, Roffi M, Rutten F H, Sibbing D, Siontis GCM; ESC Scientific Document Group. 2020 ESC Guidelines for the management of acute coronary syndromes in patients presenting without persistent ST-segment elevation. Eur Heart J. 2020 Aug. 29:ehaa575. doi: 10.1093/eurheartj/ehaa575. Epub ahead of print. PMID: 32860058.
3. Azar R R, Sarkis A, Giannitsis E. A Practical Approach for the Use of High-Sensitivity Cardiac Troponin Assays in the Evaluation of Patients With Chest Pain. Am J Cardiol. 2021 Jan. 15; 139:1-7. doi: 10.1016/j.amjcard.2020.10.037. Epub 2020 Oct. 24. PMID: 33164762.
4. Giannitsis E, Gopi V. Biomarkers for infarct diagnosis and rapid rule-out/rule-in of acute myocardial infarction. Herz. 2020 September; 45(6):509-519. English. doi: 10.1007/s00059-020-04943-x. PMID: 32468140.
5. Chiang C H, Chiang C H, Lee G H, Gi W T, Wu Y K, Huang S S, Yeo Y H, Giannitsis E, Lee C C. Safety and efficacy of the European Society of Cardiology 0/1-hour algorithm for diagnosis of myocardial infarction: systematic review and meta-analysis. Heart. 2020 July; 106(13):985-991. doi: 10.1136/heartjn1-2019-316343. Epub 2020 Apr. 3. PMID: 32245882.
6. Roffi M, Patrono C, Collet J P, Mueller C, Valgimigli M, Andreotti F, Bax J J, Borger M A, Brotons C, Chew D P, Gencer B, Hasenfuss G, Kjeldsen K, Lancellotti P, Landmesser U, Mehilli J, Mukherjee D, Storey R F, Windecker S; ESC Scientific Document Group. 2015 ESC Guidelines for the management of acute coronary syndromes in patients presenting without persistent ST-segment elevation: Task Force for the Management of Acute Coronary Syndromes in Patients Presenting without Persistent ST-Segment Elevation of the European Society of Cardiology (ESC). Eur Heart J. 2016 Jan. 14; 37(3):267-315. doi: 10.1093/eurheartj/ehv320. Epub 2015 August 29. PMID: 26320110.
7. Neumann J T, Twerenbold R, Ojeda F, Sorensen N A, Chapman A R, Shah ASV, Anand A, Boeddinghaus J, Nestelberger T, Badertscher P, Mokhtari A, Pickering J W, Troughton R W, Greenslade J, Parsonage W, Mueller-Hennessen M, Gori T, Jernberg T, Morris N, Liebetrau C, Hamm C, Katus H A, Minzel T, Landmesser U, Salomaa V, Iacoviello L, Ferrario M M, Giampaoli S, Kee F, Thorand B, Peters A, Borchini R, Jorgensen T, Soderberg S, Sans S, Tunstall-Pedoe H, Kuulasmaa K, Renné T, Lackner K J, Worster A, Body R, Ekelund U, Kavsak P A, Keller T, Lindahl B, Wild P, Giannitsis E, Than M, Cullen L A, Mills N L, Mueller C, Zeller T, Westermann D, Blankenberg S. Application of High-Sensitivity Troponin in Suspected Myocardial Infarction. N Engl J Med. 2019 Jun. 27; 380(26):2529-2540. doi: 10.1056/NEJMoa1803377. PMID: 31242362.
8. Than M P, Pickering J W, Sandoval Y, Shah ASV, Tsanas A, Apple FS, Blankenberg S, Cullen L, Mueller C, Neumann J T, Twerenbold R, Westermann D, Beshiri A, Mills N L; MI3 collaborative. Machine Learning to Predict the Likelihood of Acute Myocardial Infarction. Circulation. 2019 Aug. 16; 140(11):899-909. doi: 10.1161/CIRCULATIONAHA.119.041980. Epub ahead of print. PMID: 31416346; PMCID: PMC6749969.

1. A computer-implemented method for classifying a patient with suspected acute coronary syndrome (ACS), comprising the steps of:
(a) receiving, by a processing unit, information on a first time-point at which a first sample has been obtained from the patient at presentation,
(b) providing, by the processing unit, on a display:
b1) a proposal for a second time-point at which a second sample shall be obtained from the patient, wherein the second time-point is within an interval of about 1 hour after the first time-point or an interval of about 2 hours after the first time-point, and
b2) a proposal for an ACS classification algorithm to be applied for the classification of the patient, wherein the ACS classification algorithm is based on the second time-point proposed in step b1),
(c) receiving, by the processing unit:
c1) information on a actual time-point at which the second sample has been obtained,
c2) a value for an amount of cardiac Troponin in the first sample, and
c3) a value for the amount of the cardiac Troponin in the second sample;
(d) analyzing, by the processing unit, whether the second sample has been obtained within the interval under b1), wherein the second sample is considered to have been obtained within the interval of about 1 hour, if the second sample has been obtained between 30 to 90 minutes after the first sample, and/or wherein the second sample is considered to have been obtained within the interval of about 2 hours, if the second sample has been obtained between 91 to 150 minutes after the first sample;
(e) classifying the patient, by the processing unit, wherein:
e1) classifying the patient comprises classifying the patient based on the ACS classification algorithm proposed in step b2), if the second sample has been obtained within the interval proposed in step b1), or
e2) classifying the patient comprises classifying the patient with an ACS classification algorithm which differs from the ACS classification algorithm proposed in step b2), if the sample has not been obtained within the interval proposed in step b1); and
(f) providing, by the processing unit, information on the classification of the patient on the display.

2. The method of claim 1, wherein the first and second samples are blood, serum or plasma samples.

3. The method of claim 1, wherein the second time-point proposed in step b1) is within an interval of about one hour after the first sample, and wherein the ACS classification algorithm proposed in step b2) is a 0/1 hour algorithm.

4. The method of claim 3, wherein the ACS classification algorithm in step e2) is a 0/2 hour algorithm, if the second sample has been obtained within 91 to 150 minutes after the first sample.

5. The method of claim 3, wherein the ACS classification algorithm in step e2) is a 0/3 hour algorithm, if the second sample has been obtained within 151 to 210 minutes after the first sample.

6. The method of claim 1, wherein step (b) further comprises providing, by the processing unit and on the display, information on whether a second sample is necessary or not.

7. The method of claim 1, wherein providing on the display the proposals under steps b1) and b2) comprises providing on the display the proposals under steps b1) and b2) in response to not classifying the patient based on the first sample only.

8. The method of claim 1, wherein classifying the patient comprises classifying the patient as rule-in if the patient is classified as suffering from ACS, act rule-out if the patient is classified as not suffering from ACS, or observation zone if further classification is required in order to rule in or rule out myocardial infarction.

9. The method of claim 8, wherein step (f) further comprises providing on the display information on whether a third sample is necessary for further classification of the patient.

10. The method of claim 9, wherein a third sample is necessary, if the patient is classified into the observation zone after the second sample.

11. The method of claim 10, wherein step (f) further comprises, providing, by the processing unit and on the display, a proposal for a third time point at which a third sample shall be obtained from the patient, wherein the third time point is 3 hours or later after the first sample.

12. The method of claim 11, further comprising:
(g) receiving, by the processing unit:
g1) information on the actual time-point at which the third sample has been obtained, and
g2) a value for an amount of the cardiac Troponin in the third sample.

13. The method of claim 12, further comprising:
(h) classifying, by the processing unit, the patient as rule-out or rule-in based on a difference between the value of the amount of cardiac Troponin in the third sample and the value of the amount of cardiac Troponin in the first sample.

14. (canceled)

15. (canceled)

16. The method of claim 1, wherein the patient is a human subject.

17. The method of claim 1, wherein the cardiac Troponin is cardiac Troponin T.

18. The method of claim 1, wherein the cardiac Troponin is cardiac Troponin I.

19. The method of claim 1, further comprising selecting a diagnostic protocol for the classification of the patient prior to performance of step (a).

20. The method of claim 19, further comprising providing, by the processing unit and in response to a violation of the diagnostic protocol, information corresponding to the violation on the display.

21. (canceled)

22. A classification device for classifying a patient with suspected acute coronary syndrome (ACS), the classification device comprises:
at least one processing unit; and
a computer-readable storage medium comprising instructions stored thereon which, when executed by the at least one processing unit, causes the classification device to:
receive information on a first time-point at which a first sample has been obtained from a subject at presentation;
provide on a display:
a proposed second time-point at which a second sample should be obtained from the subject, wherein the second time-point is within an interval of about 1 hour or of about 2 hours after the first time-point, and
a proposed ACS classification algorithm for classification of the subject, wherein the proposed ACS classification algorithm is based on the proposed second time-point;
receive information corresponding to an actual time-point at which the second sample is obtained from the subject;
receive a first value indicative of an amount of cardiac Troponin in the first sample;
receive a second value indicative of the amount of cardiac Troponin in the second sample;
determine that the second sample was obtained from the subject within the interval of about 1 hour if the second sample was obtained between 30 to 90 minutes after the first time-point;
determine that the second sample was obtained from the subject within the interval of about 2 hours if the second sample was obtained between 91 to 150 minutes after the first time-point;
classify the subject based on the proposed ACS classification algorithm if the second sample was obtained from the subject within the interval of about 1 hour or of about 2 hours after the first time-point;
classify the subject based on an ACS classification algorithm which differs from the proposed ACS classification algorithm if the second sample was not obtained from the subject within the interval of about 1 hour or of about 2 hours after the first time-point; and
provide classification information corresponding to the subject on the display.

23. One or more non-transitory machine-readable storage media comprising a plurality of instructions stored thereon that, in response to execution by at least one processing device, causes a computing system to:
receive information on a first time-point at which a first sample has been obtained from a subject at presentation;
provide on a display:
a proposed second time-point at which a second sample should be obtained from the subject, wherein the second time-point is within an interval of about 1 hour or of about 2 hours after the first time-point, and
a proposed acute coronary syndrome (ACS) classification algorithm for classification of the subject, wherein the proposed ACS classification algorithm is based on the proposed second time-point;
receive information corresponding to an actual time-point at which the second sample is obtained from the subject;
receive a first value indicative of an amount of cardiac Troponin in the first sample;
receive a second value indicative of the amount of cardiac Troponin in the second sample;
determine that the second sample was obtained from the subject within the interval of about 1 hour if the second sample was obtained between 30 to 90 minutes after the first time-point;
determine that the second sample was obtained from the subject within the interval of about 2 hours if the second sample was obtained between 91 to 150 minutes after the first time-point;
classify the subject based on the proposed ACS classification algorithm if the second sample was obtained from the subject within the interval of about 1 hour or of about 2 hours after the first time-point;
classify the subject based on an ACS classification algorithm which differs from the proposed ACS classification algorithm if the second sample was not obtained from the subject within the interval of about 1 hour or of about 2 hours after the first time-point; and provide classification information corresponding to the subject on the display.
